(19) United States
(12) Patent Application Publication
(10) Pub. No.: US 2011/0245249 A1
(43) Pub. Date: Oct. 6, 2011

(54) Heterocyclically Substituted Anilinopyrimidines

(75) Inventors: Pierre Wasnaire, Dusseldorf (DE); Jorg Nico Greul, Leverkusen (DE); Oliver Gaertzen, Koln (DE); Hendrik Helmke, Liederbach (DE); Stefan Hillebrand, Neuss (DE); Amos Mattes, Langenfeld (DE); Carl Friedrich Nising, Langenfeld (DE); Ulrike Wachendorff-Neumann, Neuwied (DE); Peter Dahmen, Neuss (DE); Arnd Voerste, Koln (DE); Ruth Meissner, Leverkusen (DE); Christoph Andreas Braun, Dusseldorf (DE); Martin Kaussmann, Koln (DE); Hiroyuki Hadano, Tochigi (JP)

(73) Assignee: Bayer CropScience AG, Monheim (DE)

(21) Appl. No.: 13/061,932

(22) PCT Filed: Aug. 22, 2009
(86) PCT No.: PCT/EP2009/006115

§ 371 Date: Jun. 10, 2011

(30) Foreign Application Priority Data
Sep. 3, 2008 (EP) 08163559.1

(51) Int. Cl. A01N 43/54; C07D 405/12; C07D 239/48; C07D 409/12; C07D 413/14; A01N 43/84; C07D 493/04; A01N 43/90; A01P 3/00

(52) U.S. Cl. 514/235.8; 544/323; 514/275; 544/122

(57) ABSTRACT

Heterocyclically substituted anilinopyrimidines of the formula (I)

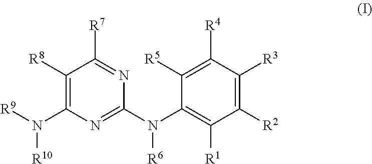

in which R¹ to R¹² and E1, E2, E3, L₁, Y, Z and L₂ have the meanings given in the description, and agrochemically active salts thereof, their use and also methods and compositions for controlling phytopathogenic harmful fungi in and/or on plants or in and/or on seed of plants, processes for preparing such compositions and treated seed and also their use for controlling phytopathogenic harmful fungi in agriculture, horticulture and forestry, in the protection of materials and in the domestic and hygiene field. The present invention furthermore relates to a process for preparing heterocyclically substituted anilinopyrimidinenes of the formula (I).

The invention relates to heterocyclically substituted diaminopyrimidines and their agrochemically active salts, to their use and to methods and compositions for controlling phytopathogenic fungi in and/or on plants or in and/or on seed of plants, to processes for preparing such compositions and treated seed and also to the use for controlling phytopathogenic harmful fungi in agriculture, horticulture and forestry, in the protection of materials and in the domestic and hygiene field. The present invention furthermore relates to a process for preparing heterocyclically substituted anilinopyrimidines.

It is already known that certain alkynyl-substituted diaminopyrimidines can be used as fungicidal crop protection agents (see DE 4029650 A1). However, in particular at low application rates, the fungicidal activity of these compounds is not always sufficient.

Since the ecological and economic demands made on modern fungicides are increasing constantly, for example with respect to activity spectrum, toxicity, selectivity, application rate, formation of residues and favourable manufacture, and there can furthermore be problems, for example, with resistance, there is a constant need to develop novel crop protection agents, in particular fungicides, which, at least in some areas, have advantages over the known crop protection agents.

Surprisingly, it has now been found that the present heterocyclyl-substituted anilinopyrimidines solve at least some aspects of the objects mentioned and are suitable for the use as crop protection agents, in particular as fungicides.

Some diaminopyrimidines are already known as pharmaceutically active components (see, for example, WO 07/140,957, WO 06/021544, WO 07/072,158, WO 07/003,596, WO 05/016893, WO 05/013996, WO 04/056807, WO 04/014382, WO 03/030909), but not their surprising fungicidal activity.

The invention provides compounds of the formula (I)

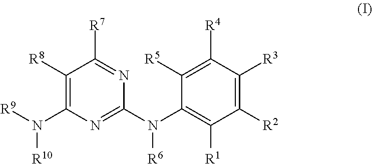

in which one or more of the symbols have one of the meanings below:
R¹ to R⁵ independently of one another represent hydrogen, OH, halogen, cyano, C₁-C₄-alkyl, C₁-C₄-alkoxy, C₁-C₄-haloalkyl, C₁-C₄-alkoxycarbonyl, NMe₂, SCF₃, SCH₃, OCF₂H or OCF₃,
   where exactly one of the radicals R² and R³ represents a group of the formula E1, E2 or E3,

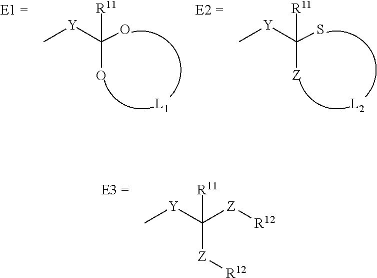

in which one or more of the symbols have one of the meanings below:
Y represents a direct bond or a C₁-C₃-alkyl chain optionally substituted by C₁-C₄-alkyl, C₁-C₃-haloalkyl or C₁-C₃-alkoxyalkyl,
Z represents sulphur or oxygen,
   with the proviso that, if R² or R³ represents a group E3, both Z represent either oxygen or sulphur,
   L₁ represents an unsubstituted or substituted C₂-C₄-alkylene chain or a CH₂CH═CHCH₂,
   where the individual carbon atoms of the alkylene chain may carry one or more substituents, independently of one another selected from the list below:
   hydrogen, OH, CH₂OH, halogen, CN, NMe₂, SCH₃, NO₂, optionally branched C₁-C₆-alkyl, optionally branched C₃-C₆-cycloalkyl, optionally branched C₁-C₄-alkoxy, optionally branched C₁-C₄-alkoxycarbonyl, optionally branched C₁-C₄-alkoxy-C₁-C₄-alkyl, optionally substituted phenyl or benzyl, optionally branched C₁-C₄-haloalkyl, optionally branched C₁-C₄-alkylmercapto-C₁-C₄-alkyl, C₁-C₄-alkylcarbonyl, CH₂O(CO)CH₃, morpholin-4-ylmethyl, pyrrolidin-1-ylmethyl, (4-methoxyphenoxy)methyl, (diethylamino)methyl, (prop-2-en-1-yloxy)methyl, (2-ethoxy-ethoxy)methyl, CH₂CN, CH₂CH₂CN, CH₂SiMe₃, 2,2-difluorocyclopropyl or methylidene,
   or
   two substituents attached to a carbon atom together with this carbon atom form a 3- to 6-membered unsubstituted or substituted saturated carbocycle,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   two substituents attached to two adjacent carbon atoms together with these two carbon atoms form
   a 5- to 8-membered unsubstituted or substituted saturated carbocycle,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   a 5- or 6-membered unsubstituted or substituted saturated heterocycle which contains an oxygen atom or a sulphur atom,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   an unsubstituted or substituted phenyl ring,
   where the substituents independently of one another are selected from the list below:
   hydrogen, halogen, CN, SCH₃, NO₂, optionally branched C₁-C₄-alkyl, optionally branched C₁-C₄-alkoxy, optionally branched C₁-C₄-alkylcarbonyl, optionally branched C₁-C₄-haloalkyl, optionally branched C₁-C₄-haloalkoxy,
   L₂ is an unsubstituted or substituted C₂- to C₃-alkylene chain,
   where the individual carbon atoms may carry one or more substituents independently of one another selected from the list below:
   hydrogen, CH₂OH, optionally branched C₁-C₄-alkyl, optionally branched C₁-C₄-alkoxy, optionally branched C₁-C₄-alkoxy-C₁-C₄-alkyl, optionally branched C₁-C₄-carbonyloxy-C₁-C₄-alkyl, optionally branched C₁-C₄-alkoxycarbonyl, branched C₁-C₄-haloalkyl, CH₂O(CO)CH₃, CH₂O(CO)CH₂CH₃, phenyl or benzyl,
R¹¹ represents hydrogen, CH₂OH, optionally branched C₁-C₄-alkyl, optionally branched C₁-C₄-alkoxy-C₁-C₄-alkyl, optionally branched C₁-C₄-haloalkyl, C₁-C₄-alkoxycarbonyl-methyl, optionally branched C₃-C₆-cycloalkyl, CH₂CN, CH₂CH₂CN, (2-methyl-1,3-dioxan-2-yl)-methyl, thiophen-2-yl, CH₂CH₂OH, phenyl or CH₂Ph,
R¹² represents branched or straight-chain C₁-C₅-alkyl, CH₂CH₂SH, prop-2-en-1-yl, COCH₃, COCH₂CH₃ or CH₂Ph,
R⁶ represents hydrogen, methyl, C₁-C₄-alkylcarbonyl, CHO, C₁-C₄-alkoxy-C₁-C₄-alkylcarbonyl, C₁-C₄-alkoxy-C₁-C₄-alkyl, optionally branched C₁-C₄-alkoxycarbonyl, C₁-C₄-haloalkylcarbonyl, C₁-C₃-alkenyl, C₁-C₃-alkynyl, C₁-C₄-alkylsulphinyl, C₁-C₄-alkylsulphonyl, optionally substituted benzyl, C₁-C₄-trialkylsilyl, C₁-C₄-trialkylsilylethyl or C₁-C₄-dialkylmonophenylsilyl,
   where the substituents independently of one another are selected from the group consisting of hydrogen, methyl, fluorine, chlorine or bromine, C₁-C₄-alkyl, C₁-C₄-alkoxy, hydroxyl, C₁-C₄-haloalkyl and cyano,
R⁷ represents hydrogen, cyano, C₁-C₃-alkyl or C₁-C₃-haloalkyl,
R⁸ represents halogen, cyano, CF₃, methyl, CFH₂, CF₂H, CCl₃, SMe, S(O)Me or SO₂Me,
R⁹ represents hydrogen, straight-chain or branched C₁-C₃-alkyl, 2-methoxyethan-1-yl, prop-2-en-1-yl, C₁-C₄-alkoxy(C₁-C₄)alkyl, straight-chain or branched (C₁-C₄-alkyl)carbonyl, (C₁-C₄-haloalkyl)carbonyl, unsubstituted or substituted benzyl, C₁-C₆-trialkylsilyl, C₁-C₄-trialkylsilylethyl, C₁-C₄-dialkylmonophenylsilyl, (C₁-C₄-alkoxy)carbonyl, C₁-C₆-alkylsulphinyl, C₁-C₆-alkylsulphonyl, C₁-C₆-haloalkylsulphinyl or C₁-C₆-haloalkylsulphonyl,
   where the substituents independently of one another are selected from the group consisting of hydrogen, halogen, nitro, C₁-C₄-alkyl, C₁-C₄-alkoxy, hydroxyl, C₁-C₄-haloalkyl and cyano,
R¹⁰ represents straight-chain or branched unsubstituted or substituted C₁-C₇-alkyl, straight-chain or branched unsubstituted or substituted C₂-C₇-haloalkyl, unsubstituted or substituted C₃-C₇-cycloalkyl, straight-chain or branched unsubstituted or substituted C₃-C₇-cycloalkyl(C₁-C₃)alkyl, straight-chain or branched unsubstituted or substituted C₃-C₇-alkenyl, straight-chain or branched unsubstituted or substituted C₃-C₇-alkynyl, straight-chain or branched unsubstituted or substituted C₁-C₄-alkoxy(C₁-C₄)alkyl, straight-chain or branched unsubstituted or substituted C₁-C₄-haloalkoxy(C₁-C₄)alkyl, 2-methyl-1-(methylsulphanyl)propan-2-yl or oxetan-3-yl,
   or
R⁹ and R¹⁰ together with the nitrogen atom to which they are attached form an unsubstituted or substituted 3- to 7-membered saturated cycle which may contain up to one further heteroatom selected from the group consisting of oxygen, sulphur and nitrogen,
   where the substituents R¹⁰ independently of one another are selected from the group consisting of methyl, ethyl, isopropyl, cyclopropyl, fluorine, chlorine and/or bromine atoms, methoxy, ethoxy, methylmercapto, ethylmercapto, cyano, hydroxyl and CF₃,
   and also agrochemically active salts thereof.

The invention further relates to the use of the compounds of the formula (I) as fungicides.

The diaminopyrimidines of the formula (I) according to the invention and their agrochemically active salts are highly suitable for controlling phytopathogenic harmful fungi. The compounds according to the invention mentioned above have in particular strong fungicidal activity and can be used both in crop protection, in the domestic and hygiene field and in the protection of materials.

The compounds of the formula (I) can be present both in pure form and as mixtures of various possible isomeric forms, in particular of stereoisomers, such as E and Z, threo and erythro, and also optical isomers, such as R and S isomers or atropisomers, and, if appropriate, also of tautomers. What is claimed are both the E and the Z isomers, and the threo and erythro, and also the optical isomers, any mixtures of these isomers, and also the possible tautomeric forms.

Preference is given to compounds of the formula (I) in which one or more of the symbols have one of the meanings below:
R¹ to R⁵ independently of one another represent hydrogen, OH, OCF₃, halogen, OMe, SCF₃, methyl, ethyl, CF₃, CF₂CF₃, CO₂Me, CO₂Et, SCH₃ or OCF₂H,
   where exactly one of the radicals R² and R³ represents a group of the formula E1, E2 or E3

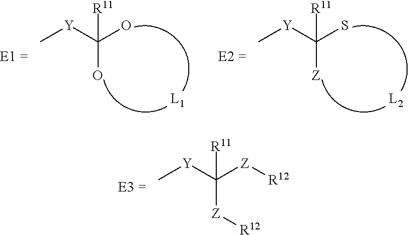

in which one or more of the symbols have one of the meanings below:
Y represents a direct bond or —CH₂—, —CH₂CH₂—, —CHMe—, —CHEt-, —CH(CH₃)CH₂—, —CH₂CH(CH₃)— or —CHOMe—,
Z represents sulphur or oxygen,
   with the proviso that, if R² or R³ represents a group E3, both Z represent either oxygen or sulphur,
   L₁ represents an unsubstituted or substituted C₂- to C₃-alkylene, where the individual carbon atoms may carry one or more substituents independently of one another selected from the list below:
   hydrogen, OH, methyl, ethyl, propyl, butyl, pentyl, hexyl, phenyl, isopropyl, tert-butyl, CH₂CH(CH₃)₂, CH₂C(CH₃)₃, 2-nitrophenyl, 2-chlorophenyl, 4-chlorophenyl, 4-methylphenyl, 4-aminophenyl, chloromethyl, CH₂CH₂Cl, CH₂F, CH₂Br, CH₂CH₂Br, hydroxymethyl, CH₂O(CO)CCH₃, methoxymethyl, morpholin-4-ylmethyl, pyrrolidin-1-ylmethyl, (4-methoxyphenoxy)methyl, (diethylamino)methyl, (prop-2-en-1-yloxy)methyl, CO₂Me, CO₂Et, COOPr, COOBu, CF₃, NO₂, CH₂SCH₃, CH₂SCH₂CH₃, CH₂CH₂SCH₃, CH₂CH₂SCH₂CH₃, CH₂CH₂OCH₃, CH₂OCH₂CH₃, CH₂CH₂OCH₂CH₃, CH₂OCH₂CH₂CH₃, (2-ethoxyethoxy)methyl, cyclopropyl, cyclobutyl, cyclopentyl, cyclohexyl, CH₂CN, CH₂CH₂CN, CH₂SiMe₃, 2,2-difluoro-cyclopropyl, CH₂OCMe₃, methylidene, chlorine, fluorine, bromine, iodine, OCH₃, OCH₂CH₃, OCH₂Ph, OC(CH₃)₃, OCH(CH₃)₂, O-propyl, O-butyl, COCH₃ or COCH₂CH₃,
   or
   two substituents attached to a carbon atom together with this carbon atom form a 3- to 6-membered unsubstituted or substituted saturated carbocycle,
   where the substituents independently of one another are selected from the list below:
   hydrogen, methyl, ethyl, propyl, isopropyl, tert-butyl, methoxy, ethoxy, propoxy, CF₃, CHF₂,
   or
   two substituents attached to two adjacent carbon atoms together with these two carbon atoms form
   a 5- to 8-membered unsubstituted or substituted saturated carbocycle,
   where the substituents independently of one another are selected from the list below:
   hydrogen, fluorine, methyl, ethyl, propyl, isopropyl, tert-butyl, methoxy, ethoxy, propoxy, CF₃, CHF₂,
   or
   a 5- or 6-membered unsubstituted or substituted saturated heterocycle which contains one oxygen atom or one sulphur atom,
   where the substituents independently of one another are selected from the list below:
   hydrogen, fluorine, methyl, ethyl, propyl, isopropyl, tert-butyl, methoxy, ethoxy, propoxy, CF₃, CHF₂,
   or
   an unsubstituted or substituted phenyl ring,
   where the substituents independently of one another are selected from the list below:
   hydrogen, chlorine, fluorine, CN, NO₂, methyl, ethyl, propyl, isopropyl, tert-butyl, methoxy, ethoxy, propoxy, CO—CH₃, COCH₂CH₃, CF₃, CHF₂, OCF₃, OCHF₂,
   L₂ is an unsubstituted or substituted C₂- to C₃-alkylene, where the individual carbon atoms may carry one or more substituents independently of one another selected from the list below:
   hydrogen, methyl, ethyl, propyl, isopropyl, butyl, CH₂OH, OCH₂CH₃, methoxymethyl, CH₂OCH₂CH₃, CH₂O(CO)CCH₃, CH₂O(CO)CH₂CH₃, CH₂Br, CH₂Cl, CO₂Me, CO₂Et, phenyl or tert-butyl,
R¹¹ represents hydrogen, methyl, ethyl, propyl, isopropyl, tert-butyl, CH₂OH, CH₂OCH₃, CH₂OCH₂CH₃, CH₂CF₃, CH₂CN, CH₂CH₂CN, (2-methyl-1,3-dioxan-2-yl)methyl, CH₂Cl, CH₂Br, CH₂F, CHBrCH₃, CH₂CO₂CH₃, CH₂CO₂CH₂CH₃, CH₂COOPr, CH₂COOBu, thiophen-2-yl, cyclopropyl, cyclohexyl, CH₂CH₂OH, CH₂CH₂OCH₃, phenyl or CH₂Ph,
R¹² represents methyl, ethyl, propyl, butyl, isopropyl, CH₂CH(CH₃)₂, CH₂C(CH₃)₃, CH₂CH₂SH, prop-2-en-1-yl, COCH₃ or CH₂Ph,
R⁶ represents hydrogen, methyl, COMe, CHO, COCH₂OCH₃, CH₂OCH₃, COOMe or COOEt, COOtertBu, COOBn, COCF₃, CH₂CH═CH₂, CH₂CCH, SOCH₃, SO₂CH₃ or benzyl,
R⁷ represents hydrogen, cyano, methyl, CF₃ or CFH₂,
R⁸ represents halogen, cyano, CF₃, methyl, CFH₂, CF₂H, CCl₃, SMe, S(O)Me or SO₂Me,
R⁹ represents hydrogen, methyl, ethyl, propyl, propan-2-yl, 2-methoxyethan-1-yl, prop-2-en-1-yl, CH₂OCH₃, COMe, COOMe, COOEt, COOtertBu, COCF₃ or benzyl,
R¹⁰ represents straight-chain or branched unsubstituted or substituted C₁-C₆-alkyl, straight-chain or branched unsubstituted or substituted C₃-C₆-cycloalkyl(C₁-C₂)alkyl, unsubstituted or substituted C₃-C₆-cycloalkyl, straight-chain or branched unsubstituted or substituted C₃-C₄-alkenyl, straight-chain or branched unsubstituted or substituted C₃-C₄-alkynyl, straight-chain or branched unsubstituted or substituted C₂-C₄-haloalkyl, straight-chain or branched unsubstituted or substituted C₁-C₂-alkoxy(C₁-C₄)alkyl, straight-chain or branched unsubstituted or substituted C₁-C₂-alkylmercapto-(C₁-C₄)alkyl or oxetan-3-yl,
   where the substituents in R¹⁰ independently of one another are selected from the group consisting of methyl, ethyl, isopropyl, cyclopropyl, fluorine, chlorine and/or bromine atoms, methoxy, ethoxy, methylmercapto, ethylmercapto, cyano, hydroxyl and CF₃,
   or
   R⁹ and R¹⁰ together with the nitrogen atom to which they are attached form an azetidinyl, pyrrolidinyl, piperidinyl, morpholinyl, azepanyl, 4-methylpiperazin-1-yl, 2-methylpiperidin-1-yl, -methylpyrrolidin-1-yl, 2-methylazetidin-1-yl or thiomorpholinyl ring,
   and also agrochemically active salts thereof.

Particular preference is given to compounds of the formula (I) in which one or more of the symbols have one of the meanings below:

R¹ to R⁵ independently of one another represent hydrogen, OH, OCF₃, halogen, OMe, SCF₃, methyl, ethyl, CF₃, CF₂CF₃ or CO₂Me,
where exactly one of the radicals R² and R³ represents a group of the formula E1, E2 or E3,

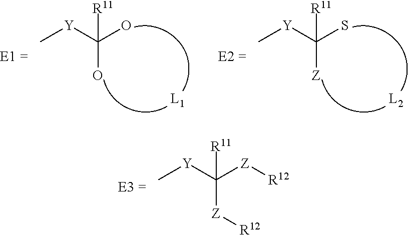

in which one or more of the symbols have one of the meanings below:
Y represents a direct bond or —CH₂—, —CH₂CH₂— or —CHMe—,
Z represents sulphur or oxygen,
with the proviso that, if R² or R³ represents a group E3, both Z represent either oxygen or sulphur,
L₁ represents an unsubstituted or substituted C₂- to C₃-alkylene, where the individual carbon atoms may carry one or more substituents independently of one another selected from the list below:
hydrogen, methyl, ethyl, propyl, butyl, pentyl, hexyl, phenyl, isopropyl, tert-butyl, 2-nitrophenyl, 2-chlorophenyl, 4-chlorophenyl, 4-methylphenyl, 4-aminophenyl, chloromethyl, CH₂F, CH₂Br, hydroxymethyl, CH₂O(CO)CCH₃, methoxymethyl, morpholin-4-ylmethyl, pyrrolidin-1-ylmethyl, (4-methoxyphenoxy)methyl, (diethylamino)methyl, (prop-2-en-1-yloxy)methyl, CO₂Me, CO₂Et, CF₃, NO₂, CH₂SCH₃, CH₂SCH₂CH₃, CH₂CH₂SCH₃, CH₂CH₂SCH₂CH₃, CH₂CH₂OCH₃, CH₂OCH₂CH₃, CH₂CH₂OCH₂CH₃, CH₂OCH₂CH₂CH₃, (2-ethoxyethoxy)methyl, cyclopropyl, cyclopentyl, cyclohexyl, CH₂CN, CH₂CH₂CN, CH₂SiMe₃, 2,2-difluorocyclopropyl, CH₂OCMe₃, methylidene, chlorine, fluorine, bromine, iodine, OCH₃, OCH₂CH₃, OCH₂Ph or COCH₃,
or
two substituents attached to a carbon atom together with this carbon atom form a 3- to 6-membered unsubstituted or substituted saturated carbocycle,
where the substituents independently of one another are selected from the list below:
hydrogen, methyl, ethyl, propyl, methoxy, ethoxy or CF₃,
or
two substituents attached to two adjacent carbon atoms together with these two carbon atoms form
a 5- or 6-membered unsubstituted or substituted saturated carbocycle,
where the substituents independently of one another are selected from the list below:
hydrogen, fluorine, methyl, ethyl, propyl, methoxy, ethoxy or CF₃,
or
a 5- or 6-membered unsubstituted or substituted saturated heterocycle which contains an oxygen atom or a sulphur atom,
where the substituents independently of one another are selected from the list below:
hydrogen, fluorine, methyl, ethyl, tert-butyl, methoxy, ethoxy or CF₃,
or
an unsubstituted or substituted phenyl ring,
where the substituents independently of one another are selected from the list below:
hydrogen, chlorine, fluorine, CN, methyl, ethyl, tert-butyl, methoxy, CF₃ or CO—CH₃,
L₂ represents an unsubstituted or substituted C₂- to C₃-alkylene, where the individual carbon atoms may carry one or more substituents independently of one another selected from the list below:
hydrogen, methyl, ethyl, propyl, isopropyl, butyl, hydroxymethyl, methoxymethyl, CH₂O(CO)CCH₃, CH₂Br, CO₂Me, phenyl or tert-butyl,
R¹¹ represents hydrogen, methyl, ethyl, propyl, isopropyl, tert-butyl, CH₂OCH₃, CH₂CF₃, CH₂CN, CH₂CH₂CN, (2-methyl-1,3-dioxan-2-yl)methyl, CH₂Cl, CH₂Br, CHBrCH₃, CH₂CO₂CH₃, CH₂CO₂CH₂CH₃, thiophen-2-yl or cyclopropyl,
R¹² represents methyl, ethyl, propyl, butyl, isopropyl, CH₂CH₂SH, prop-2-en-1-yl, COCH₃ or CH₂Ph,
R⁶ represents hydrogen, methyl, COMe, CHO or COCH₂OCH₃,
R⁷ represents hydrogen,
R⁸ represents cyano, CF₃, methyl, CFH₂, CF₂H, CCl₃, SMe, S(O)Me or SO₂Me,
R⁹ represents hydrogen, methyl, ethyl, propyl, propan-2-yl, 2-methoxyethan-1-yl or prop-2-en-1-yl,
R¹⁰ represents methyl, ethyl, propyl, cyclopropyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, 2-butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, prop-2-yn-1-yl, butan-2-yl, 2,2,2-trifluoroethyl, 2,2-difluoroethyl, 2-methoxyethan-1-yl, 2-methyl-mercaptoethan-1-yl, 2-fluoroethan-1-yl, 2-chloroethan-1-yl, 2-cyanoethan-1-yl, 1-methoxypropan-2-yl, 3-methoxypropan-1-yl, 2-hydroxyethan-1-yl, 1-hydroxypropan-2-yl, 3-hydroxypropan-1-yl, 1-methylmercaptopropan-2-yl, 2-methyl-1-(methylsulphanyl)-propan-2-yl, oxetan-3-yl, 1,1,1-trifluoropropan-2-yl, 2,2,3,3,3-pentafluoropropyl, 1,1,1-tri-fluoropropan-3-yl, 1,1,1-trifluorobutan-2-yl, 1,1,1-trifluorobutan-3-yl, 2-methylprop-2-en-1-yl, 1-fluoropropan-2-yl, 2-methylcyclobut-1-yl or 3-methylcyclobut-1-yl,
   or
R⁹ and R¹⁰ together with the nitrogen atom to which they are attached represent an azetidinyl, pyrrolidinyl, piperidinyl, morpholinyl, azepanyl, 4-methylpiperazin-1-yl, 2-methylpiperidin-1-yl, -methylpyrrolidin-1-yl, 2-methylazetidin-1-yl or thiomorpholinyl ring,
   and also agrochemically active salts thereof.

Very particular preference is given to compounds of the formula (I)

in which one or more of the symbols have one of the meanings below:
R¹ and R⁵ represent hydrogen,
R² represents hydrogen,
or represents a group of the formula E1, E2 or E3

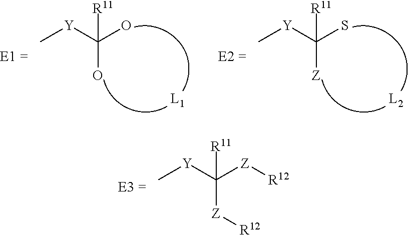

in which one or more of the symbols have one of the meanings below:
Y represents a direct bond or —CH₂— or —CH₂CH₂—,
Z represents sulphur or oxygen,
with the proviso that, if R² or R³ represents a group E3, both Z represent either oxygen or sulphur,
L₁ represents an unsubstituted or substituted C₂- to C₃-alkylene, where the individual carbon atoms may carry one or more substituents independently of one another selected from the list below:
hydrogen, methyl, ethyl, propyl, butyl, pentyl, hexyl, phenyl, tert-butyl, 2-nitrophenyl, chloromethyl, CH₂F, hydroxymethyl, methoxymethyl, morpholin-4-ylmethyl, pyrrolidin-1-ylmethyl, (4-methoxyphenoxy)methyl, (diethylamino)methyl, (prop-2-en-1-yloxy)methyl, CO₂Me, CF₃, CH₂SCH₃, CH₂SCH₂CH₃, CH₂OCH₂CH₃, (2-ethoxyethoxy)methyl, cyclopropyl, CH₂CN, CH₂SiMe₃, 2,2-difluorocyclopropyl, CH₂OCMe₃, methylidene, chlorine, fluorine, OCH₃, OCH₂CH₃, OCH₂Ph or COCH₃,
or
two substituents attached to a carbon atom together with this carbon atom form a 5- to 6-membered unsubstituted saturated carbocycle,
or
two substituents attached to two adjacent carbon atoms together with these two carbon atoms form
a 5- or 6-membered unsubstituted saturated carbocycle,
or
a 5- or 6-membered unsubstituted saturated heterocycle which contains one oxygen atom,
or
an unsubstituted phenyl ring,
L₂ represents an unsubstituted or substituted C₂- to C₃-alkylene chain, where the individual carbon atoms may carry one or more substituents independently of one another selected from the list below:
hydrogen, methyl, ethyl or propyl,
R³ represents hydrogen, halogen, OMe or methyl,
or represents a group of the formula E1, E2 or E3,

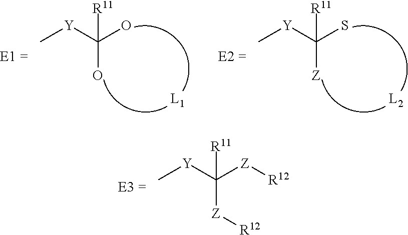

in which one or more of the symbols have one of the meanings below:
Y represents a direct bond or —CH₂—, —CH₂CH₂—,
Z represents sulphur or oxygen,
with the proviso that, if R² or R³ represents a group E3, both Z represent either oxygen or sulphur,
L₁ represents an unsubstituted or substituted C₂- to C₃-alkylene, where the individual carbon atoms may carry one or more substituents independently of one another selected from the list below:
methyl, ethyl or methoxymethyl,
or
two substituents attached to a carbon atom together with this carbon atom form a 5- to 6-membered unsubstituted saturated carbocycle,
or
two substituents attached to two adjacent carbon atoms together with these two carbon atoms form
a 5- or 6-membered unsubstituted saturated carbocycle,
or
a 5- or 6-membered unsubstituted saturated heterocycle which contains one oxygen atom,
or
an unsubstituted phenyl ring,
L₂ represents an unsubstituted or substituted C₂- to C₃-alkylene, where the individual carbon atoms may carry one or more substituents independently of one another selected from the list below:
hydrogen, methyl, ethyl or propyl,
R¹¹ represents hydrogen, methyl, ethyl, propyl, CH₂OCH₃, CH₂CF₃, CH₂CN or (2-methyl-1,3-dioxan-2-yl)methyl,
R¹² represents methyl, ethyl, propyl, butyl, isopropyl or CH₂CH₂SH,
   where R² and R³ are not both hydrogen,
   with the proviso that, if R² is not hydrogen,
   R³ can only have one of the meanings below:
   hydrogen, chlorine, bromine, fluorine, iodine, OMe or methyl,
R⁴ represents hydrogen, halogen, OMe, SCF₃, methyl, ethyl, CF₃, CF₂CF₃ or CO₂Me,
R⁶ represents hydrogen,
R⁷ represents hydrogen,
R⁸ represents chlorine, bromine, fluorine, iodine, cyano, CF₃, methyl, SMe, S(O)Me or SO₂Me,
R⁹ represents hydrogen, methyl, ethyl, propyl or propan-2-yl,
R¹⁰ represents methyl, cyclopropyl, cyclopropylmethyl, 1-cyclopropyleth-1-yl, 2-methylcyclopropyl, cyclobutyl, 1-fluoroprop-2-yl, cyclopentyl, propan-2-yl, pentyl, prop-2-en-1-yl, prop-2-yn-1-yl, 2,2,2-trifluoroethyl, 2,2-difluoroethyl, 2-methoxyethan-1-yl, 2-fluoroethan-1-yl, 2-chloroethan-1-yl, 1-methoxypropan-2-yl or 2-hydroxyethan-1-yl,
   or
R⁹ and R¹⁰ together with the nitrogen atom to which they are attached form an azetidinyl, pyrrolidinyl, piperidinyl, morpholinyl, azepanyl, or thiomorpholinyl ring,
   and also agrochemically active salts thereof.

Special preference is given to compounds of the formula (I)

in which one or more of the symbols have one of the meanings below:
R¹ and R⁵ represent hydrogen,
R² represents hydrogen, 1,3-dioxolan-2-yl, 4-methyl-1,3-dioxolan-2-yl, 4-ethyl-1,3-dioxolan-2-yl, 4-propyl-1,3-dioxolan-2-yl, 4-butyl-1,3-dioxolan-2-yl, 4-pentyl-1,3-dioxolan-2-yl, 4-hexyl-1,3-dioxolan-2-yl, 4-phenyl-1,3-dioxolan-2-yl, 4-tert-butyl-1,3-dioxolan-2-yl, 4-(2-nitrophenyl)-1,3-dioxolan-2-yl, 4-(chloromethyl)-1,3-dioxolan-2-yl, 4-(fluoromethyl)-1,3-dioxolan-2-yl, 4-(hydroxymethyl)-1,3-dioxolan-2-yl, 4-(methoxymethyl)-1,3-dioxolan-2-yl, 4-(morpholin-4-ylmethyl)-1,3-dioxolan-2-yl, 4-(pyrrolidin-1-ylmethyl)-1,3-dioxolan-2-yl, 4,5-dimethyl-1,3-dioxolan-2-yl, 4-[(4-methoxyphenoxy)methyl]-1,3-dioxolan-2-yl, 4-[(diethylamino)methyl]-1,3-dioxolan-2-yl, 4-[(prop-2-en-1-yloxy)methyl]-1,3-dioxolan-2-yl, hexahydro-1,3-benzodioxol-2-yl, tetrahydrofuro[3,4-d][1,3]dioxol-2-yl, (4R,5R)-4,5-bis(methoxycarbonyl)-1,3-dioxolan-2-yl, 4-(trifluoromethyl)-1,3-dioxolan-2-yl, 4-[(methylsulphanyl)methyl]-1,3-dioxolan-2-yl, 4-[(ethylsulphanyl)methyl]-1,3-dioxolan-2-yl, 4-(ethoxymethyl)-1,3-dioxolan-2-yl, 4,4,5,5-tetramethyl-1,3-dioxolan-2-yl, 1,3-benzodioxol-2-yl, 4-ethyl-4-methyl-1,3-dioxolan-2-yl, 4-[(2-ethoxyethoxy)methyl]-1,3-dioxolan-2-yl, (4R)-4-cyclopropyl-1,3-dioxolan-2-yl, (4R)-4-(cyanomethyl)-1,3-dioxolan-2-yl, 4-[(trimethylsilyl)methyl]-1,3-dioxolan-2-yl, 4-(2,2-difluorocyclopropyl)-1,3-dioxolan-2-yl, 4-(tert-butoxymethyl)-1,3-dioxolan-2-yl,
bis(2-sulphanylethoxy)methyl, bis(propan-2-yloxy)methyl, dibutoxymethyl, dipropoxymethyl, diethoxymethyl, dimethoxymethyl,
1,3-dioxolan-2-ylmethyl, 2-(1,3-dioxolan-2-yl)ethyl,
1,3-dioxan-2-yl, 2-methyl-1,3-dioxan-2-yl, 4,6-dimethyl-1,3-dioxan-2-yl, 5,5-dimethyl-1,3-dioxan-2-yl, 4-methyl-1,3-dioxan-2-yl, 5-methyl-1,3-dioxan-2-yl, 4,4-dimethyl-1,3-dioxan-2-yl, 4-phenyl-1,3-dioxan-2-yl, 4,6-dimethyl-1,3-dioxan-2-yl, 4,4,6-trimethyl-1,3-dioxan-2-yl, 4,5,6-trimethyl-1,3-dioxan-2-yl, 4,4,6,6-tetramethyl-1,3-dioxan-2-yl, 4-tert-butyl-1,3-dioxan-2-yl, 4-(trifluoromethyl)-1,3-dioxan-2-yl, 5-methylidene-1,3-dioxan-2-yl, 5-chloro-1,3-dioxan-2-yl, 5,5-difluoro-1,3-dioxan-2-yl, 5-methoxy-1,3-dioxan-2-yl, 5-ethoxy-1,3-dioxan-2-yl, 5-(benzyloxy)-1,3-dioxan-2-yl, 5-cyclopropyl-1,3-dioxan-2-yl, 5-tert-butyl-1,3-dioxan-2-yl, 5,7-dioxaspiro[2.5]oct-6-yl, 6,8-dioxaspiro[3.5]non-7-yl, 5-oxo-1,3-dioxan-2-yl, 5-acetyl-4,6-dimethyl-1,3-dioxan-2-yl, 5-acetyl-4,5,6-trimethyl-1,3-dioxan-2-yl,
1,3-dioxan-2-ylmethyl, 2-(1,3-dioxan-2-yl)ethyl,
1,3-dithiolan-2-yl, 4-methyl-1,3-dithiolan-2-yl, 4-ethyl-1,3-dithiolan-2-yl, 4,5-dimethyl-1,3-dithiolan-2-yl, 1,3-dithian-2-yl, 4-methyl-1,3-dithian-2-yl, 1,3-oxathiolan-2-yl, 5-methyl-1,3-oxathiolan-2-yl, 4,5-dimethyl-1,3-oxathiolan-2-yl 1,3-oxathian-2-yl, 4-propyl-1,3-oxathian-2-yl,
bis(methylsulphanyl)methyl, bis(ethylsulphanyl)methyl,
2-methyl-1,3-dioxolan-2-yl, 2-ethyl-1,3-dioxolan-2-yl, 2-propyl-1,3-dioxolan-2-yl, 2-(methoxymethyl)-1,3-dioxolan-2-yl, 2-(2,2,2-trifluoroethyl)-1,3-dioxolan-2-yl, 2-[(2-methyl-1,3-dioxolan-2-yl)methyl]-1,3-dioxolan-2-yl,
2,4-dimethyl-1,3-dioxolan-2-yl, 4-ethyl-2-methyl-1,3-dioxolan-2-yl, 2,4,5-trimethyl-1,3-dioxolan-2-yl, 4-(methoxymethyl)-2-methyl-1,3-dioxolan-2-yl, 2-methyltetrahydro-furo[3,4-d][1,3]dioxol-2-yl,
(2-methyl-1,3-dioxolan-2-yl)methyl, 2-(2-methyl-1,3-dioxolan-2-yl)ethyl,
2-methyl-1,3-dioxan-2-yl, 2-ethyl-1,3-dioxan-2-yl, 2-propyl-1,3-dioxan-2-yl, 2-(methoxymethyl)-1,3-dioxan-2-yl, 2-(2,2,2-trifluoroethyl)-1,3-dioxan-2-yl, 2-[(2-methyl-1,3-dioxan-2-yl)methyl]-1,3-dioxan-2-yl, 2,4-dimethyl-1,3-dioxan-2-yl, 2,4,6-trimethyl-1,3-dioxan-2-yl, 2,5,5-trimethyl-1,3-dioxan-2-yl,
1,1-dimethoxyethyl, 1,1-diethoxyethyl,
(2-methyl-1,3-dioxan-2-yl)methyl, 2-(2-methyl-1,3-dioxan-2-yl)ethyl,
2-methyl-1,3-dithiolan-2-yl, 2-methyl-1,3-oxathiolan-2-yl, 2-methyl-1,3-dithian-2-yl, 2-methyl-1,3-oxathian-2-yl,
R³ represents hydrogen, chlorine, bromine, fluorine, iodine, OMe, methyl, 1,3-dioxolan-2-yl, 4-methyl-1,3-dioxolan-2-yl, 4-ethyl-1,3-dioxolan-2-yl, 4,5-dimethyl-1,3-dioxolan-2-yl, 4-(methoxymethyl)-1,3-dioxolan-2-yl, tetrahydrofuro[3,4-d][1,3]dioxol-2-yl,
1,3-dioxolan-2-ylmethyl
1,3-dioxan-2-yl, 2-methyl-1,3-dioxan-2-yl, 4,6-dimethyl-1,3-dioxan-2-yl, 5,5-dimethyl-1,3-dioxan-2-yl,
1,3-dioxan-2-ylmethyl,
1,3-dithiolan-2-yl, 1,3-oxathiolan-2-yl, 1,3-dithian-2-yl, 1,3-oxathian-2-yl,
2-methyl-1,3-dioxolan-2-yl, 2-ethyl-1,3-dioxolan-2-yl, 2-(2,2,2-trifluoroethyl)-1,3-dioxan-2-yl, 2-[(2-methyl-1,3-dioxolan-2-yl)methyl]-1,3-dioxolan-2-yl, 2-(cyanomethyl)-1,3-dioxolan-2-yl,
(2-methyl-1,3-dioxolan-2-yl)methyl,
2-methyl-1,3-dioxan-2-yl, 2-ethyl-1,3-dioxan-2-yl, 2-(2,2,2-trifluoroethyl)-1,3-dioxan-2-yl,
2-[(2-methyl-1,3-dioxan-2-yl)methyl]-1,3-dioxan-2-yl, 2-(cyanomethyl)-1,3-dioxan-2-yl,
(2-methyl-1,3-dioxan-2-yl)methyl,
2-methyl-1,3-dithiolan-2-yl, 2-methyl-1,3-oxathiolan-2-yl, 2-methyl-1,3-dithian-2-yl, 2-methyl-1,3-oxathian-2-yl,
   where R² and R³ are not both hydrogen,
   with the proviso that, if R² is not hydrogen,
R³ can only have one of the meanings below:
   hydrogen, chlorine, bromine, fluorine, iodine, OMe or methyl,
R⁴ represents hydrogen, chlorine, bromine, fluorine, iodine, OMe, SCF₃, methyl, ethyl, CF₃, CF₂CF₃ or CO₂Me,
R⁶ represents hydrogen,
R⁷ represents hydrogen,
R⁸ represents chlorine, bromine, fluorine, iodine, cyano, CF₃, methyl, SMe, S(O)Me or SO₂Me,
R⁹ represents hydrogen or methyl,
R¹⁰ represents cyclopropyl, cyclopropylmethyl, cyclobutyl, propan-2-yl, prop-2-yn-1-yl, 2,2,2-trifluorethyl, 2,2-difluorethyl or 1-methoxypropan-2-yl,
   or
R⁹ and R¹⁰ together with the nitrogen atom to which they are attached represent an azetidinyl, pyrrolidinyl, piperidinyl or morpholinyl ring,
   and also agrochemically active salts thereof.

Especial preference is furthermore given to compounds of the formula (I)

in which one or more of the symbols have one of the meanings below:
R¹ and R⁵ each represent hydrogen,
R² represents hydrogen, 1,3-dioxolan-2-yl, 4-methyl-1,3-dioxolan-2-yl, 4-ethyl-1,3-dioxolan-2-yl, 4-butyl-1,3-dioxolan-2-yl, 4-hexyl-1,3-dioxolan-2-yl, 4-phenyl-1,3-dioxolan-2-yl, 4-tert-butyl-1,3-dioxolan-2-yl, 4-(2-nitrophenyl)-1,3-dioxolan-2-yl, 4-(hydroxymethyl)-1,3-dioxolan-2-yl, 4-(methoxymethyl)-1,3-dioxolan-2-yl, 4-(morpholin-4-ylmethyl)-1,3-dioxolan-2-yl, 4-(pyrrolidin-1-ylmethyl)-1,3-dioxolan-2-yl, 4,5-dimethyl-1,3-dioxolan-2-yl, 4-[(4-methoxyphenoxy)methyl]-1,3-dioxolan-2-yl,
bis(2-sulphanylethoxy)methyl, bis(propan-2-yloxy)methyl, dibutoxymethyl, diethoxymethyl, dimethoxymethyl,
1,3-dioxan-2-yl, 2-methyl-1,3-dioxan-2-yl, 4,6-dimethyl-1,3-dioxan-2-yl, 5,5-dimethyl-1,3-dioxan-2-yl,
1,3-dithiolan-2-yl, 1,3-dithian-2-yl, 1,3-oxathiolan-2-yl or
2-methyl-1,3-dioxolan-2-yl,
R³ represents hydrogen, chlorine or 2-methyl-1,3-dioxan-2-yl,
   where R² and R³ do not both represent hydrogen, with the proviso that, if R² does not represent hydrogen,
R³ can only have one of the meanings below:
   hydrogen or chlorine,
R⁴ represents hydrogen,
R⁶ represents hydrogen,
R⁷ represents hydrogen,
R⁸ represents chlorine, bromine, fluorine, iodine, CF₃ or methyl,
R⁹ represents hydrogen,
R¹⁰ represents cyclopropyl, cyclopropylmethyl, cyclobutyl, prop-2-yn-1-yl, 2,2,2-trifluoroethyl, 2,2-difluoroethyl or 1-methoxypropan-2-yl,
   and also agrochemically active salts thereof.

Preference is furthermore given to compounds of the formula (I) in which

the radical R² represents a group of the formula E1, E2 or E3

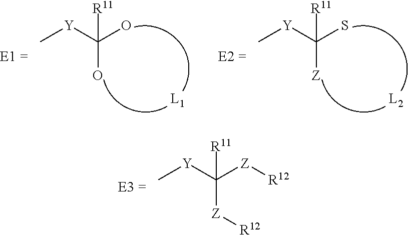

in which one or more of the symbols have one of the meanings below:
Y represents a direct bond or —CH₂— or —CH₂CH₂—,
Z represents sulphur or oxygen,
with the proviso that, if R² or R³ represents a group E3, both Z represent either oxygen or represent sulphur,
L₁ represents an unsubstituted or substituted C₂- to C₃-alkylene, where the individual carbon atoms may carry one or more substituents independently of one another selected from the list below:
hydrogen, methyl, ethyl, propyl, butyl, pentyl, hexyl, phenyl, tert-butyl, 2-nitrophenyl, chloromethyl, CH₂F, CF₃, hydroxymethyl, methoxymethyl, morpholin-4-ylmethyl, pyrrolidin-1-ylmethyl, (4-methoxyphenoxy)methyl, (diethylamino)methyl, (prop-2-en-1-yloxy)methyl, CO₂Me, CF₃, CH₂SCH₃, CH₂SCH₂CH₃, CH₂OCH₂CH₃, (2-ethoxyethoxy)methyl, cyclopropyl, CH₂CN, CH₂SiMe₃, 2,2-difluorocyclopropyl, CH₂OCMe₃, methylidene, chlorine, fluorine, OCH₃, OCH₂CH₃, OCH₂Ph or COCH₃,
or
two substituents attached to a carbon atom together with this carbon atom form a 3- to 6-membered unsubstituted saturated carbocycle
or
two substituents attached to two adjacent carbon atoms together with these two carbon atoms form
a 5- or 6-membered unsubstituted saturated carbocycle,
or
a 5- or 6-membered unsubstituted saturated heterocycle which contains an oxygen atom
or
an unsubstituted phenyl ring,
L₂ represents an unsubstituted or substituted C₂- to C₃-alkylene chain where the individual carbon atoms may carry one or more substituents independently of one another selected from the list below:
hydrogen, methyl, ethyl or propyl, CH₂OH,
where the other substituents have one or more of the meanings mentioned above,
and the agrochemically active salts thereof.

Preference is furthermore given to compounds of the formula (I) in which

the radical R² represents a group of the formula E1, E2 or E3

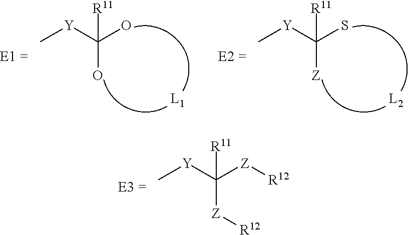

in which one or more of the symbols have one of the meanings below:
Y represents a direct bond or a C₁-C₃-alkyl chain optionally substituted by C₁-C₄-alkyl, C₁-C₃-haloalkyl or C₁-C₃-alkoxyalkyl,
Z represents sulphur or oxygen,
L₁ represents an unsubstituted or substituted C₂-C₄-alkylene chain or CH₂CH═CHCH₂,

where the individual carbon atoms of the alkylene chain may carry one or more substituents independently of one another selected from the list below:

hydrogen, OH, CH₂OH, halogen, CN, NMe₂, SCH₃, NO₂, optionally branched C₁-C₄-alkyl, optionally branched C₃-C₆-cycloalkyl, optionally branched C₁-C₄-alkoxy, optionally branched C₁-C₄-alkoxycarbonyl, optionally branched C₁-C₄-alkoxy-C₁-C₄-alkyl, optionally substituted phenyl or benzyl, optionally branched C₁-C₄-haloalkyl, optionally branched C₁-C₄-alkylmercapto-C₁-C₄-alkyl, C₁-C₄-alkylcarbonyl, CH₂O(CO)CH₃, morpholin-4-ylmethyl, pyrrolidin-1-ylmethyl, (4-methoxyphenoxy)methyl, (diethylamino)methyl, (prop-2-en-1-yloxy)methyl, (2-ethoxy-ethoxy)methyl, CH₂CN, CH₂CH₂CN, CH₂SiMe₃, 2,2-difluorocyclopropyl or methylidene,
or
two substituents attached to a carbon atom together with this carbon atom form a 3- to 6-membered unsubstituted or substituted saturated carbocycle,
where the substituents independently of one another are selected from the list below:
hydrogen, fluorine, optionally branched C₁-C₄-alkyl, optionally branched C₁-C₄-alkoxy or optionally branched C₁-C₄-haloalkyl,
or
two substituents attached to two adjacent carbon atoms of the alkylene chain together with these two carbon atoms form
a 5- to 8-membered unsubstituted or substituted saturated carbocycle,
where the substituents independently of one another are selected from the list below:
hydrogen, fluorine, optionally branched C₁-C₄-alkyl, optionally branched C₁-C₄-alkoxy or optionally branched C₁-C₄-haloalkyl,
or
a 5- or 6-membered unsubstituted or substituted saturated heterocycle which contains an oxygen atom or a sulphur atom,
where the substituents independently of one another are selected from the list below:
hydrogen, fluorine, optionally branched C₁-C₄-alkyl, optionally branched C₁-C₄-alkoxy or optionally branched C₁-C₄-haloalkyl,
or
an unsubstituted or substituted phenyl ring,
where the substituents independently of one another are selected from the list below:
hydrogen, halogen, CN, SCH₃, NO₂, optionally branched C₁-C₄-alkyl, optionally branched C₁-C₄-alkoxy, optionally branched C₁-C₄-alkylcarbonyl, optionally branched C₁-C₄-haloalkyl or optionally branched C₁-C₄-haloalkoxy,
L₂ represents an unsubstituted or substituted C₂- to C₃-alkylene chain,
where the individual carbon atoms may carry one or more substituents independently of one another selected from the list below:
hydrogen, CH₂OH, optionally branched C₁-C₄-alkyl, optionally branched C₁-C₄-alkoxy, optionally branched C₁-C₄-alkoxy-C₁-C₄-alkyl, optionally branched C₁-C₄-alkoxycarbonyl, optionally branched C₁-C₄-haloalkyl, CH₂O(CO)CH₃, CH₂O(CO)CH₂CH₃, phenyl or benzyl,
where the other substituents have one or more of the meanings mentioned above,
and also the agrochemically active salts thereof.

Preference is furthermore given to compounds of the formula (I) in which

the radical R³ represents a group of the formula E1, E2 or E3

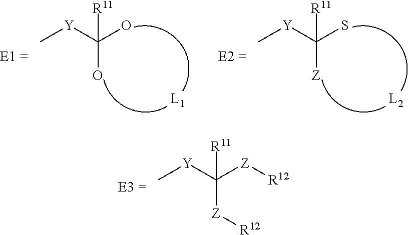

in which one or more of the symbols have one of the meanings below:
Y represents a direct bond or a C₁-C₃-alkyl chain optionally substituted by C₁-C₄-alkyl, C₁-C₃-haloalkyl or C₁-C₃-alkoxyalkyl,
Z represents sulphur or oxygen,
L₁ represents an unsubstituted or substituted C₂-C₄-alkylene chain or CH₂CH═CHCH₂,
   where the individual carbon atoms of the alkylene chain may carry one or more substituents independently of one another selected from the list below:
   hydrogen, OH, CH₂OH, halogen, CN, NMe₂, SCH₃, NO₂, optionally branched C₁-C₄-alkyl, optionally branched C₃-C₆-cycloalkyl, optionally branched C₁-C₄-alkoxy, optionally branched C₁-C₄-alkoxycarbonyl, optionally branched C₁-C₄-alkoxy-C₁-C₄-alkyl, optionally substituted phenyl or benzyl, optionally branched C₁-C₄-haloalkyl, optionally branched C₁-C₄-alkylmercapto-C₁-C₄-alkyl, C₁-C₄-alkylcarbonyl, CH₂O(CO)CH₃, morpholin-4-ylmethyl, pyrrolidin-1-ylmethyl, (4-methoxyphenoxy)methyl, (diethylamino)methyl, (prop-2-en-1-yloxy)methyl, (2-ethoxyethoxy)methyl, CH₂CN, CH₂CH₂CN, CH₂SiMe₃, 2,2-difluorocyclopropyl or methylidene,
   or
   two substituents attached to a carbon atom together with this carbon atom form a 3- to 6-membered unsubstituted or substituted saturated carbocycle,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   two substituents attached to two adjacent carbon atoms together with these two carbon atoms form
   a 5- to 8-membered unsubstituted or substituted saturated carbocycle,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   a 5- or 6-membered unsubstituted or substituted saturated heterocycle which contains an oxygen atom or a sulphur atom,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   an unsubstituted or substituted phenyl ring,
   where the substituents independently of one another are selected from the list below:
   hydrogen, halogen, CN, SCH₃, NO₂, optionally branched C₁-C₄-alkyl, optionally branched C₁-C₄-alkoxy, optionally branched C₁-C₄-alkylcarbonyl, optionally branched C₁-C₄-haloalkyl or optionally branched C₁-C₄-haloalkoxy,
   L₂ represents an unsubstituted or substituted C₂- to C₃-alkylene chain,
   where the individual carbon atoms may carry one or more substituents independently of one another selected from the list below:
   hydrogen, CH₂OH, optionally branched C₁-C₄-alkyl, optionally branched C₁-C₄-alkoxy, optionally branched C₁-C₄-alkoxy-C₁-C₄-alkyl, optionally branched C₁-C₄-alkoxycarbonyl, branched C₁-C₄-haloalkyl, CH₂O(CO)CH₃, CH₂O(CO)CH₂CH₃, phenyl or benzyl,
   where the other substituents have one or more of the meanings mentioned above,
   and also the agrochemically active salts thereof.

Preference is furthermore given to compounds of the formula (I) in which

exactly one of the radicals R² and R³ represents a group of the formula E1

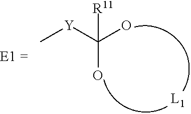

in which one or more of the symbols have one of the meanings below:
Y represents a direct bond or a C₁-C₃-alkyl chain optionally substituted by C₁-C₄-alkyl, C₁-C₃-haloalkyl or C₁-C₃-alkoxyalkyl,
   L₁ represents an unsubstituted or substituted C₂-C₄-alkylene chain or CH₂CH═CHCH₂, where the individual carbon atoms of the alkylene chain may carry one or more substituents independently of one another selected from the list below:
   hydrogen, OH, CH₂OH, halogen, CN, NMe₂, SCH₃, NO₂, optionally branched C₁-C₄-alkyl, optionally branched C₃-C₆-cycloalkyl, optionally branched C₁-C₄-alkoxy, optionally branched C₁-C₄-alkoxycarbonyl, optionally branched C₁-C₄-alkoxy-C₁-C₄-alkyl, optionally substituted phenyl or benzyl, optionally branched C₁-C₄-haloalkyl, optionally branched C₁-C₄-alkylmercapto-C₁-C₄-alkyl, C₁-C₄-alkylcarbonyl, CH₂O(CO)CH₃, morpholin-4-ylmethyl, pyrrolidin-1-ylmethyl, (4-methoxyphenoxy)methyl, (diethylamino)methyl, (prop-2-en-1-yloxy)methyl, (2-ethoxyethoxy)methyl, CH₂CN, CH₂CH₂CN, CH₂SiMe₃, 2,2-difluorocyclopropyl or methylidene,
   or
   two substituents attached to a carbon atom together with this carbon atom form a 3- to 6-membered unsubstituted or substituted saturated carbocycle,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   two substituents attached to two adjacent carbon atoms together with these two carbon atoms form
   a 5- to 8-membered unsubstituted or substituted saturated carbocycle,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   a 5- or 6-membered unsubstituted or substituted saturated heterocycle which contains an oxygen atom or a sulphur atom,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   an unsubstituted or substituted phenyl ring,
   where the substituents independently of one another are selected from the list below:
   hydrogen, halogen, CN, SCH₃, NO₂, optionally branched C₁-C₄-alkyl, optionally branched C₁-C₄-alkoxy, optionally branched C₁-C₄-alkylcarbonyl, optionally branched C₁-C₄-haloalkyl or optionally branched C₁-C₄-haloalkoxy,
   where the other substituents have one or more of the meanings mentioned above,
   and also the agrochemically active salts thereof.

Preference is furthermore given to compounds of the formula (I) in which exactly one of the radicals R² and R³ represents a group of the formula E2

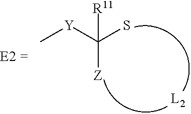

in which one or more of the symbols have one of the meanings below:
Y represents a direct bond or a C₁-C₃-alkyl chain optionally substituted by C₁-C₄-alkyl, C₁-C₃-haloalkyl or C₁-C₃-alkoxyalkyl,
Z represents sulphur or oxygen,
L₂ represents an unsubstituted or substituted C₂- to C₃-alkylene chain,
   where the individual carbon atoms may carry one or more substituents independently of one another selected from the list below:
   hydrogen, CH₂OH, optionally branched C₁-C₄-alkyl, optionally branched C₁-C₄-alkoxy, optionally branched C₁-C₄-alkoxy-C₁-C₄-alkyl, optionally branched C₁-C₄-alkoxycarbonyl, branched C₁-C₄-haloalkyl, CH₂O(CO)CH₃, CH₂O(CO)CH₂CH₃, phenyl or benzyl,
   where the other substituents have one or more of the meanings mentioned above,
   and also agrochemically active salts thereof.

Preference is furthermore given to compounds of the formula (I) in which exactly one of the radicals R² and R³ represents a group of the formula E3

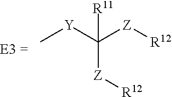

in which one or more of the symbols have one of the meanings below:
Y represents a direct bond or a C₁-C₃-alkyl chain optionally substituted by C₁-C₄-alkyl, C₁-C₃-haloalkyl or C₁-C₃-alkoxyalkyl,
Z represents sulphur or oxygen,
   where the other substituents have one or more of the meanings mentioned above,
   and also the agrochemically active salts thereof.

Preference is furthermore given to compounds of the formula (I) in which

R² represents one of the radicals below:
1,3-dioxolan-2-yl, 4-methyl-1,3-dioxolan-2-yl, 4-ethyl-1,3-dioxolan-2-yl, 4-propyl-1,3-dioxolan-2-yl, 4-butyl-1,3-dioxolan-2-yl, 4-pentyl-1,3-dioxolan-2-yl, 4-hexyl-1,3-dioxolan-2-yl, 4-phenyl-1,3-dioxolan-2-yl, 4-tert-butyl-1,3-dioxolan-2-yl, 4-(2-nitrophenyl)-1,3-dioxolan-2-yl, 4-(chloromethyl)-1,3-dioxolan-2-yl, 4-(fluoromethyl)-1,3-dioxolan-2-yl, 4-(hydroxymethyl)-1,3-dioxolan-2-yl, 4-(methoxymethyl)-1,3-dioxolan-2-yl, 4-(morpholin-4-ylmethyl)-1,3-dioxolan-2-yl, 4-(pyrrolidin-1-ylmethyl)-1,3-dioxolan-2-yl, 4,5-dimethyl-1,3-dioxolan-2-yl, 4-[(4-methoxyphenoxy)methyl]-1,3-dioxolan-2-yl, 4-[(diethylamino)methyl]-1,3-dioxolan-2-yl, 4-[(prop-2-en-1-yloxy)methyl]-1,3-dioxolan-2-yl, hexahydro-1,3-benzodioxol-2-yl, tetrahydrofuro[3,4-d][1,3]dioxol-2-yl, (4R,5R)-4,5-bis(methoxycarbonyl)-1,3-dioxolan-2-yl, 4-(trifluoromethyl)-1,3-dioxolan-2-yl, 4-[(methylsulphanyl)methyl]-1,3-dioxolan-2-yl, 4-[(ethylsulphanyl)methyl]-1,3-dioxolan-2-yl, 4-(ethoxymethyl)-1,3-dioxolan-2-yl, 4,4,5,5-tetramethyl-1,3-dioxolan-2-yl, 1,3-benzodioxol-2-yl, 4-ethyl-4-methyl-1,3-dioxolan-2-yl, 4-[(2-ethoxyethoxy)methyl]-1,3-dioxolan-2-yl, (4R)-4-cyclopropyl-1,3-dioxolan-2-yl, (4R)-4-(cyanomethyl)-1,3-dioxolan-2-yl, 4-[(trimethylsilyl)methyl]-1,3-dioxolan-2-yl, 4-(2,2-difluorocyclopropyl)-1,3-dioxolan-2-yl, 4-(tert-butoxymethyl)-1,3-dioxolan-2-yl,
bis(2-sulphanylethoxy)methyl, bis(propan-2-yloxy)methyl, dibutoxymethyl, dipropoxymethyl, diethoxymethyl, dimethoxymethyl,
1,3-dioxolan-2-ylmethyl, 2-(1,3-dioxolan-2-yl)ethyl,
1,3-dioxan-2-yl, 2-methyl-1,3-dioxan-2-yl, 4,6-dimethyl-1,3-dioxan-2-yl, 5,5-dimethyl-1,3-dioxan-2-yl, 4-methyl-1,3-dioxan-2-yl, 5-methyl-1,3-dioxan-2-yl, 4,4-dimethyl-1,3-dioxan-2-yl, 4-phenyl-1,3-dioxan-2-yl, 4,6-dimethyl-1,3-dioxan-2-yl, 4,4,6-trimethyl-1,3-dioxan-2-yl, 4,5,6-trimethyl-1,3-dioxan-2-yl, 4,4,6,6-tetramethyl-1,3-dioxan-2-yl, 4-tert-butyl-1,3-dioxan-2-yl, 4-(trifluoromethyl)-1,3-dioxan-2-yl, 5-methylidene-1,3-dioxan-2-yl, 5-chloro-1,3-dioxan-2-yl, 5,5-difluoro-1,3-dioxan-2-yl, 5-methoxy-1,3-dioxan-2-yl, 5-ethoxy-1,3-dioxan-2-yl, 5-(benzyloxy)-1,3-dioxan-2-yl, 5-cyclopropyl-1,3-dioxan-2-yl, 5-tert-butyl-1,3-dioxan-2-yl, 5,7-dioxaspiro[2.5]oct-6-yl, 6,8-dioxaspiro[3.5]non-7-yl, 5-oxo-1,3-dioxan-2-yl, 5-acetyl-4,6-dimethyl-1,3-dioxan-2-yl, 5-acetyl-4,5,6-trimethyl-1,3-dioxan-2-yl,
1,3-dioxan-2-ylmethyl, 2-(1,3-dioxan-2-yl)ethyl,
1,3-dithiolan-2-yl, 4-methyl-1,3-dithiolan-2-yl, 4-ethyl-1,3-dithiolan-2-yl, 4,5-dimethyl-1,3-dithiolan-2-yl, 1,3-dithian-2-yl, 4-methyl-1,3-dithian-2-yl, 1,3-oxathiolan-2-yl, 5-methyl-1,3-oxathiolan-2-yl, 4,5-dimethyl-1,3-oxathiolan-2-yl 1,3-oxathian-2-yl, 4-propyl-1,3-oxathian-2-yl,
bis(methylsulphanyl)methyl, bis(ethylsulphanyl)methyl,
2-methyl-1,3-dioxolan-2-yl, 2-ethyl-1,3-dioxolan-2-yl, 2-propyl-1,3-dioxolan-2-yl, 2-(methoxymethyl)-1,3-dioxolan-2-yl, 2-(2,2,2-trifluoroethyl)-1,3-dioxolan-2-yl, 2-[(2-methyl-1,3-dioxolan-2-yl)methyl]-1,3-dioxolan-2-yl,
2,4-dimethyl-1,3-dioxolan-2-yl, 4-ethyl-2-methyl-1,3-dioxolan-2-yl, 2,4,5-trimethyl-1,3-dioxolan-2-yl, 4-(methoxymethyl)-2-methyl-1,3-dioxolan-2-yl, 2-methyltetrahydro-furo[3,4-d][1,3]dioxol-2-yl,
(2-methyl-1,3-dioxolan-2-yl)methyl, 2-(2-methyl-1,3-dioxolan-2-yl)ethyl,
2-methyl-1,3-dioxan-2-yl, 2-ethyl-1,3-dioxan-2-yl, 2-propyl-1,3-dioxan-2-yl, 2-(methoxymethyl)-1,3-dioxan-2-yl, 2-(2,2,2-trifluoroethyl)-1,3-dioxan-2-yl, 2-[(2-methyl-1,3-dioxan-2-yl)methyl]-1,3-dioxan-2-yl, 2,4-dimethyl-1,3-dioxan-2-yl, 2,4,6-trimethyl-1,3-dioxan-2-yl, 2,5,5-trimethyl-1,3-dioxan-2-yl,
1,1-dimethoxyethyl, 1,1-diethoxyethyl,
(2-methyl-1,3-dioxan-2-yl)methyl, 2-(2-methyl-1,3-dioxan-2-yl)ethyl,
2-methyl-1,3-dithiolan-2-yl, 2-methyl-1,3-oxathiolan-2-yl, 2-methyl-1,3-dithian-2-yl, 2-methyl-1,3-oxathian-2-yl,
   where the other substituents have one or more of the meanings mentioned above,
   and also the agrochemically active salts thereof.

Preference is furthermore given to compounds of the formula (I) in which

R³ represents one of the radicals below:
1,3-dioxolan-2-yl, 4-methyl-1,3-dioxolan-2-yl, 4-ethyl-1,3-dioxolan-2-yl, 4-propyl-1,3-dioxolan-2-yl, 4-butyl-1,3-dioxolan-2-yl, 4-pentyl-1,3-dioxolan-2-yl, 4-hexyl-1,3-dioxolan-2-yl, 4-phenyl-1,3-dioxolan-2-yl, 4-tert-butyl-1,3-dioxolan-2-yl, 4-(2-nitrophenyl)-1,3-dioxolan-2-yl, 4-(chloromethyl)-1,3-dioxolan-2-yl, 4-(fluoromethyl)-1,3-dioxolan-2-yl, 4-(hydroxymethyl)-1,3-dioxolan-2-yl, 4-(methoxymethyl)-1,3-dioxolan-2-yl, 4-(morpholin-4-ylmethyl)-1,3-dioxolan-2-yl, 4-(pyrrolidin-1-ylmethyl)-1,3-dioxolan-2-yl, 4,5-dimethyl-1,3-dioxolan-2-yl, 4-[(4-methoxyphenoxy)methyl]-1,3-dioxolan-2-yl, 4-[(diethylamino)methyl]-1,3-dioxolan-2-yl, 4-[(prop-2-en-1-yloxy)methyl]-1,3-dioxolan-2-yl, hexahydro-1,3-benzodioxol-2-yl, tetrahydrofuro[3,4-][1,3]dioxol-2-yl, (4R,5R)-4,5-bis(methoxycarbonyl)-1,3-dioxolan-2-yl, 4-(trifluoromethyl)-1,3-dioxolan-2-yl, 4-[(methylsulphanyl)methyl]-1,3-dioxolan-2-yl, 4-[(ethylsulphanyl)methyl]-1,3-dioxolan-2-yl, 4-(ethoxymethyl)-1,3-dioxolan-2-yl, 4,4,5,5-tetramethyl-1,3-dioxolan-2-yl, 1,3-benzodioxol-2-yl, 4-ethyl-4-methyl-1,3-dioxolan-2-yl, 4-[(2-ethoxyethoxy)methyl]-1,3-dioxolan-2-yl, (4R)-4-cyclopropyl-1,3-dioxolan-2-yl, (4R)-4-(cyanomethyl)-1,3-dioxolan-2-yl, 4-[(trimethylsilyl)methyl]-1,3-dioxolan-2-yl, 4-(2,2-difluorocyclopropyl)-1,3-dioxolan-2-yl, 4-(tert-butoxymethyl)-1,3-dioxolan-2-yl,
bis(2-sulphanylethoxy)methyl, bis(propan-2-yloxy)methyl, dibutoxymethyl, dipropoxymethyl, diethoxymethyl, dimethoxymethyl,
1,3-dioxolan-2-ylmethyl, 2-(1,3-dioxolan-2-yl)ethyl,
1,3-dioxan-2-yl, 2-methyl-1,3-dioxan-2-yl, 4,6-dimethyl-1,3-dioxan-2-yl, 5,5-dimethyl-1,3-dioxan-2-yl, 4-methyl-1,3-dioxan-2-yl, 5-methyl-1,3-dioxan-2-yl, 4,4-dimethyl-1,3-dioxan-2-yl, 4-phenyl-1,3-dioxan-2-yl, 4,6-dimethyl-1,3-dioxan-2-yl, 4,4,6-trimethyl-1,3-dioxan-2-yl, 4,5,6-trimethyl-1,3-dioxan-2-yl, 4,4,6,6-tetramethyl-1,3-dioxan-2-yl, 4-tert-butyl-1,3-dioxan-2-yl, 4-(trifluoromethyl)-1,3-dioxan-2-yl, 5-methyliden-1,3-dioxan-2-yl, 5-chloro-1,3-dioxan-2-yl, 5,5-difluoro-1,3-dioxan-2-yl, 5-methoxy-1,3-dioxan-2-yl, 5-ethoxy-1,3-dioxan-2-yl, 5-(benzyloxy)-1,3-dioxan-2-yl, 5-cyclopropyl-1,3-dioxan-2-yl, 5-tert-butyl-1,3-dioxan-2-yl, 5,7-dioxaspiro[2.5]oct-6-yl, 6,8-dioxaspiro[3.5]non-7-yl, 5-oxo-1,3-dioxan-2-yl, 5-acetyl-4,6-dimethyl-1,3-dioxan-2-yl, 5-acetyl-4,5,6-trimethyl-1,3-dioxan-2-yl,
1,3-dioxan-2-ylmethyl, 2-(1,3-dioxan-2-yl)ethyl,
1,3-dithiolan-2-yl, 4-methyl-1,3-dithiolan-2-yl, 4-ethyl-1,3-dithiolan-2-yl, 4,5-dimethyl-1,3-dithiolan-2-yl, 1,3-dithian-2-yl, 4-methyl-1,3-dithian-2-yl, 1,3-oxathiolan-2-yl, 5-methyl-1,3-oxathiolan-2-yl, 4,5-dimethyl-1,3-oxathiolan-2-yl 1,3-oxathian-2-yl, 4-propyl-1,3-oxathian-2-yl,
bis(methylsulphanyl)methyl, bis(ethylsulphanyl)methyl,
2-methyl-1,3-dioxolan-2-yl, 2-ethyl-1,3-dioxolan-2-yl, 2-propyl-1,3-dioxolan-2-yl, 2-(methoxymethyl)-1,3-dioxolan-2-yl, 2-(2,2,2-trifluoroethyl)-1,3-dioxolan-2-yl, 2-[(2-methyl-1,3-dioxolan-2-yl)methyl]-1,3-dioxolan-2-yl,
2,4-dimethyl-1,3-dioxolan-2-yl, 4-ethyl-2-methyl-1,3-dioxolan-2-yl, 2,4,5-trimethyl-1,3-dioxolan-2-yl, 4-(methoxymethyl)-2-methyl-1,3-dioxolan-2-yl, 2-methyltetrahydro-furo[3,4-d][1,3]dioxol-2-yl,
(2-methyl-1,3-dioxolan-2-yl)methyl, 2-(2-methyl-1,3-dioxolan-2-yl)ethyl,
2-methyl-1,3-dioxan-2-yl, 2-ethyl-1,3-dioxan-2-yl, 2-propyl-1,3-dioxan-2-yl, 2-(methoxymethyl)-1,3-dioxan-2-yl, 2-(2,2,2-trifluoroethyl)-1,3-dioxan-2-yl, 2-[(2-methyl-1,3-dioxan-2-yl)methyl]-1,3-dioxan-2-yl, 2,4-dimethyl-1,3-dioxan-2-yl, 2,4,6-trimethyl-1,3-dioxan-2-yl, 2,5,5-trimethyl-1,3-dioxan-2-yl,
1,1-dimethoxyethyl, 1,1-diethoxyethyl,
(2-methyl-1,3-dioxan-2-yl)methyl, 2-(2-methyl-1,3-dioxan-2-yl)ethyl,
2-methyl-1,3-dithiolan-2-yl, 2-methyl-1,3-oxathiolan-2-yl, 2-methyl-1,3-dithian-2-yl, 2-methyl-1,3-oxathian-2-yl,
   where the other substituents have one or more of the meanings mentioned above,
   and also the agrochemically active salts thereof.

Preference is furthermore given to compounds of the formula (I) in which

R¹ and R⁵ both represent hydrogen,
where the other substituents have one or more of the meanings mentioned above,
and also the agrochemically active salts thereof.

Preference is furthermore given to compounds of the formula (I) in which

R⁶ represents hydrogen,
where the other substituents have one or more of the meanings mentioned above,
and also the agrochemically active salts thereof.

Preference is furthermore given to compounds of the formula (I) in which

R⁷ represents hydrogen,
where the other substituents have one or more of the meanings mentioned above,
and also the agrochemically active salts thereof.

Preference is furthermore given to compounds of the formula (I) in which

R⁸ represents chlorine, bromine, fluorine, iodine, cyano, CF₃, methyl,
where the other substituents have one or more of the meanings mentioned above,
and also the agrochemically active salts thereof.

Preference is furthermore given to compounds of the formula (I) in which

R⁹ represents hydrogen or methyl,
where the other substituents have one or more of the meanings mentioned above,
and also the agrochemically active salts thereof.

Preference is furthermore given to compounds of the formula (I) in which

R¹, R⁵, R⁶ and R⁷ represent hydrogen,
where the other substituents have one or more of the meanings mentioned above,
and also the agrochemically active salts thereof.

Preference is furthermore given to compounds of the formula (I) in which
R¹⁰ represents cyclopropyl, cyclopropylmethyl, cyclobutyl, propan-2-yl, prop-2-yn-1-yl, 2,2,2-trifluoroethyl, 2,2-difluoroethyl or 1-methoxypropan-2-yl,
   where the other substituents have one or more of the meanings mentioned above,
   and also the agrochemically active salts thereof.

The radical definitions mentioned above can be combined with one another as desired. Moreover, individual definitions may not apply.

Examples of inorganic acids are hydrohalic acids, such as hydrogen fluoride, hydrogen chloride, hydrogen bromide and hydrogen iodide, sulphuric acid, phosphoric acid and nitric acid, and acidic salts, such as NaHSO₄ and KHSO₄. Suitable organic acids are, for example, formic acid, carbonic acid and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, alkylsulphonic acids (sulphonic acids having straight-chain or branched alkyl radicals of 1 to 20 carbon atoms), arylsulphonic acids or aryldisulphonic acids (aromatic radicals, such as phenyl and naphthyl, which carry one or two sulphonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphonic acid radicals), where the alkyl and aryl radicals may carry further substituents, for example p-toluenesulphonic acid, salicylic acid, p-aminosalicylic acid, 2-phenoxybenzoic acid, 2-acetoxybenzoic acid, etc.

Suitable metal ions are in particular the ions of the elements of the second main group, in particular calcium and magnesium, of the third and fourth main group, in particular aluminium, tin and lead, and also of the first to eighth transition group, in particular chromium, manganese, iron, cobalt, nickel, copper, zinc and others. Particular preference is given to the metal ions of the elements of the fourth period. Here, the metals can be present in various valencies that they can assume.

Optionally substituted groups may be mono- or polysubstituted, where in the case of polysubstitution the substituents may be identical or different.

In the definitions of the symbols given in the formulae above, collective terms were used which are generally representative for the following substituents:

halogen: fluorine, chlorine, bromine and iodine;
aryl: an unsubstituted or optionally substituted 5- to 15-membered partially or fully unsaturated mono-, bi- or tricyclic ring system having up to 3 ring members selected from the groups C(═O), (C═S), where at least one of the rings of the ring system is fully unsaturated, such as, for example (but not limited thereto) benzene, naphthalene, tetrahydronaphthalene, anthracene, indane, phenanthrene, azulene;
alkyl: saturated straight-chain or branched hydrocarbon radicals having 1 to 10 carbon atoms, such as, for example (but not limited thereto) methyl, ethyl, propyl, 1-methylethyl, butyl, 1-methyl-propyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, heptyl, 1-methylhexyl, octyl, 1,1-dimethylhexyl, 2-ethylhexyl, 1-ethylhexyl, nonyl, 1,2,2-trimethylhexyl, decyl;
haloalkyl: straight-chain or branched alkyl groups having 1 to 4 carbon atoms (as mentioned above), where in these groups some or all of the hydrogen atoms may be replaced by halogen atoms as mentioned above, such as, for example (but not limited thereto), C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2-difluoroethyl, 2,2-dichloro-2-fluoroethyl, 2,2,2-trichloroethyl, pentafluoroethyl and 1,1,1-trifluoroprop-2-yl;
alkenyl: unsaturated straight-chain or branched hydrocarbon radicals having 2 to 16 carbon atoms and at least one double bond in any position, such as, for example (but not limited thereto), C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
alkynyl: straight-chain or branched hydrocarbon groups having 2 to 16 carbon atoms and at least one triple bond in any position, such as, for example (but not limited thereto), C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
alkoxy: saturated straight-chain or branched alkoxy radicals having 1 to 4 carbon atoms, such as, for example (but not limited thereto), C₁-C₄-alkoxy, such as methoxy, ethoxy, propoxy, 1-methylethoxy, butoxy, 1-methylpropoxy, 2-methylpropoxy, 1,1-dimethylethoxy;
haloalkoxy: straight-chain or branched alkoxy groups having 1 to 4 carbon atoms (as mentioned above), where some or all of the hydrogen atoms in these groups may be replaced by halogen atoms as mentioned above, such as, for example (but not limited thereto), C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoro-ethoxy and 1,1,1-trifluoroprop-2-oxy;
thioalkyl: saturated straight-chain or branched alkylthio radicals having 1 to 6 carbon atoms, such as, for example (but not limited thereto), C₁-C₆-alkylthio, such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio;
thiohaloalkyl: straight-chain or branched alkylthio groups having 1 to 6 carbon atoms (as mentioned above), where some or all of the hydrogen atoms in these groups may be replaced by halogen atoms as mentioned above, such as, for example (but not limited thereto) C₁-C₂-haloalkylthio, such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio;
cycloalkyl: mono-, bi- or tricyclic saturated hydrocarbon groups having 3 to 12 carbon ring members, such as, for example (but not limited thereto), cyclopropyl, cyclobutyl, cyclopentyl and cyclohexyl, bicyclo[1.0.1]butane, decalinyl, norbornyl;
cycloalkenyl: mono-, bi- or tricyclic non-aromatic hydrocarbon groups having 5 to 15 carbon ring members and at least one double bond, such as, for example (but not limited thereto) cyclopenten-1-yl, cyclohexen-1-yl, cyclohepta-1,3-dien-1-yl, norbornen-1-yl;
(alkoxy)carbonyl: an alkoxy group having 1 to 4 carbon atoms (as mentioned above) which is attached to the skeleton via a carbonyl group (—CO—);
heterocyclyl: a three- to fifteen-membered saturated or partially unsaturated heterocycle which contains one to four heteroatoms from the group consisting of oxygen, nitrogen and sulphur: mono-, bi- or tricyclic heterocycles containing, in addition to carbon ring members, one to three nitrogen atoms and/or one oxygen or sulphur atom or one or two oxygen and/or sulphur atoms; if the ring contains a plurality of oxygen atoms, these are not directly adjacent; such as, for example (but not limited thereto), oxiranyl, aziridinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetra-hydropyranyl, 4-tetrahydropyranyl, 2-tetrahydrothienyl, 3-hexahydropyridazinyl, 4-hexa-hydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl;
hetaryl: unsubstituted or optionally substituted, 5- to 15-membered partially or fully unsaturated mono-, bi- or tricyclic ring system where at least one of the rings of the ring system is fully unsaturated, comprising one to four heteroatoms from the group consisting of oxygen, nitrogen and sulphur, if the ring contains a plurality of oxygen atoms, these are not directly adjacent;
such as, for example (but not limited thereto),
5-membered heteroaryl which contains one to four nitrogen atoms or one to three nitrogen atoms and one sulphur or oxygen atom: 5-membered heteroaryl groups which, in addition to carbon atoms, may contain one to four nitrogen atom or one to three nitrogen atoms and one sulphur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,446-azol-2-yl;
benzo-fused 5-membered heteroaryl which contains one to three nitrogen atoms or one nitrogen atom and one oxygen or sulphur atom: 5-membered heteroaryl groups which, in addition to carbon atoms, may contain one to four nitrogen atoms or one to three nitrogen atoms and one sulphur or oxygen atom as ring members, and in which two adjacent carbon ring members or one nitrogen and one adjacent carbon ring member may be bridged by a buta-1,3-diene-1,4-diyl group in which one or two carbon atoms may be replaced by nitrogen atoms; for example benzindolyl, benzimidazolyl, benzothiazolyl, benzopyrazolyl, benzofuryl;
5-membered heteroaryl which contains one to four nitrogen atoms and is attached via nitrogen or benzo-fused 5-membered heteroaryl which contains one to three nitrogen atoms and is attached via nitrogen: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, for example 1-pyrrolyl, 1-pyrazolyl, 1,2,4-triazol-1-yl, 1-imidazolyl, 1,2,3-triazol-1-yl, 1,3,4-triazol-1-yl;
6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

Not included are combinations which contradict natural laws and which the person skilled in the art would therefore have excluded based on his expert knowledge. Excluded are, for example, ring structures having three or more adjacent oxygen atoms.

The present invention furthermore relates to a process (A) for preparing the diaminopyrimidines of the formula (I) according to the invention

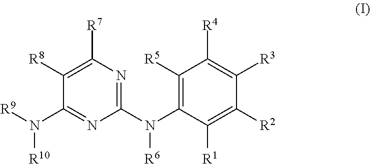

hereinbelow, depending on the appropriate process, also referred to by formula (Ia-c),

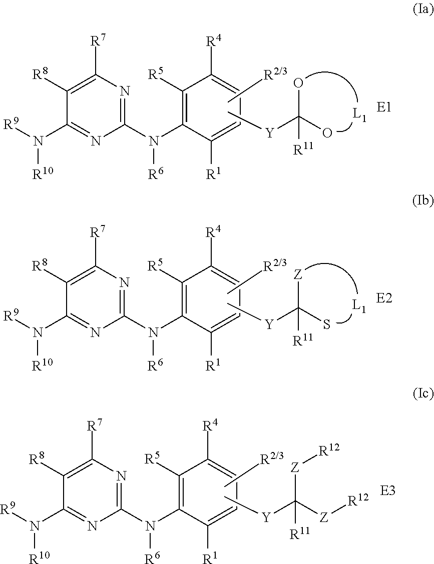

having in each case exactly one heterocyclic side chain E1, E2 or E3 in the R² or R³ position (meta or para),
comprising at least one of steps (a) to (g) below:
(a) the reaction of 2,4-dihalopyrimidines of the formula (III) with amines of the formula (II) in the presence of a base, if appropriate in the presence of a solvent, if appropriate in the presence of a catalyst, to give compounds of the formula (V), according to the reaction scheme below (Scheme 1):

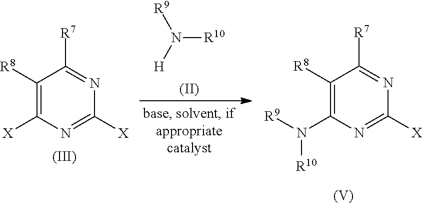

where X=F, Cl, Br, I
(b) the reaction of compounds of the formula (V) with aromatic amines of the formula (IV), if appropriate in the presence of an acid, if appropriate in the presence of a solvent, according to the reaction scheme below (Scheme 2):

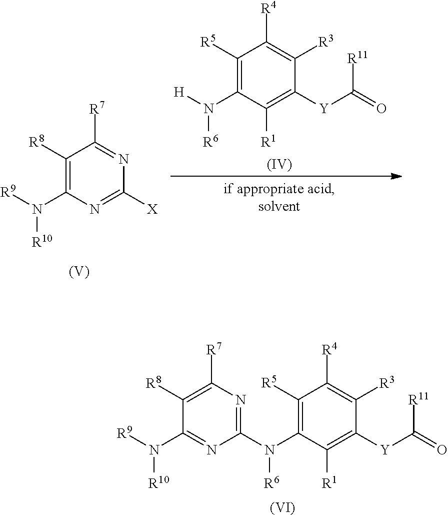

where X=F, Cl, Br, I
(c) the reaction of compounds of the formula (V) with aromatic amines of the formula (VII) if appropriate in the presence of an acid, if appropriate in the presence of a solvent, according to the reaction scheme below (Scheme 3):

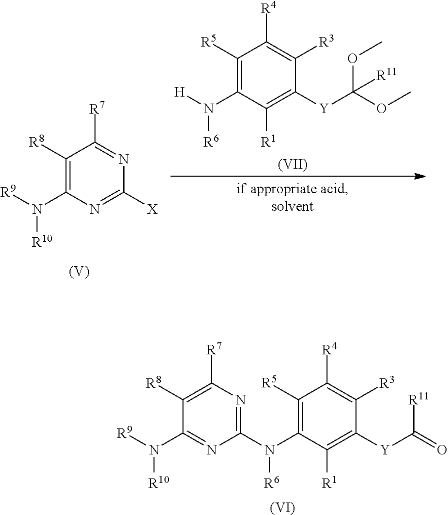

where X=F, Cl, Br, I
(d) the reaction of compounds of the formula (VI) with diols of the formula (VIII) in the presence of an acid, if appropriate in the presence of a solvent, to give compounds of the formula (Ia), according to the reaction scheme below (Scheme 4):

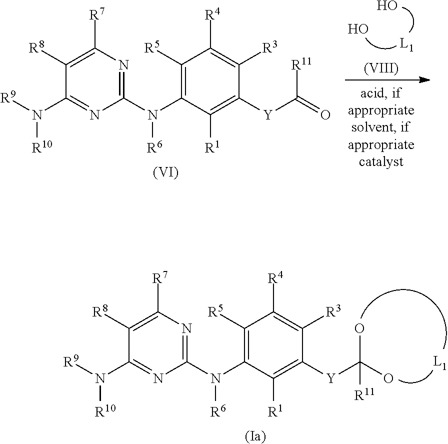

(e) The reaction of compounds of the formula (V) with anilines of the formula (IX) in the presence of an acid, if appropriate in the presence of a solvent, if appropriate in the presence of a catalyst, to give compounds of the formula (Ia), according to the reaction scheme below (Scheme 5)

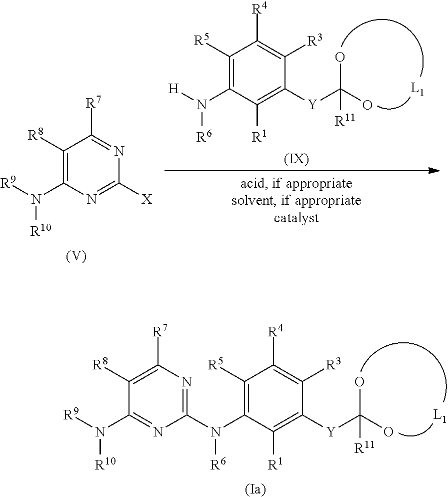

where X=F, Cl, Br, I
(f) The reaction of compounds of the formula (VI) with compounds of the formula (X) in the presence of an acid, if appropriate in the presence of a solvent, if appropriate in the presence of a catalyst, to give compounds of the formula (Ib), according to the reaction scheme below (Scheme 6)

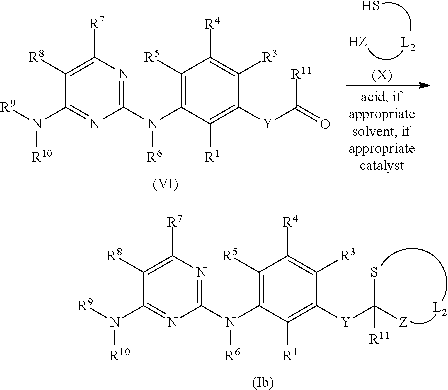

(g) The reaction of compounds of the formula (VI) with alcohols or thiols of the formula (XI) in the presence of an acid, if appropriate in the presence of a solvent, if appropriate in the presence of a catalyst, to give compounds of the formula (Ic), according to the reaction scheme below (Scheme 7)

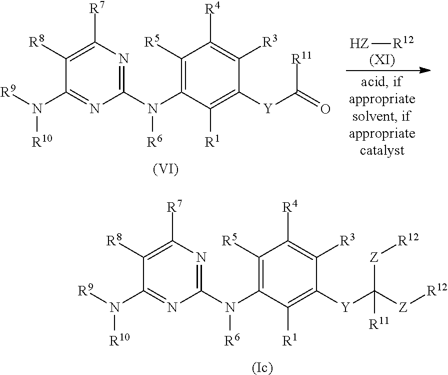

where the definitions of the radicals R¹ to R¹² and Y and Z in the above schemes correspond to the definitions given above.

One way of preparing the intermediate (V) is shown in Scheme 1.

The alkylamino compounds of the formula (II) are either commercially available or can be prepared by literature procedures. One method for preparing suitable cyclopropylamino compounds of type (II) is, for example, the rearrangement of suitable carboxylic acid derivatives to the corresponding amino compounds (described, for example, in J. Am. Chem. Soc. 1961, 83, 3671-3678). Other methods, for example for preparing cyclobutylamino compounds of type (II), comprise the hydroboration of suitable cyclobutenes and subsequent treatment with NH₂SO₃H (for example Tetrahedron 1970, 26, 5033-5039), the reductive amination of cyclobutanones (described, for example, in J. Org. Chem. 1964, 29, 2588-2592) and also the reduction of nitro- or nitrosocyclobutanes (see, for example, J. Am. Chem. Soc. 1953, 75, 4044; Can. J. Chem. 1963, 41, 863-875) or azidocyclobutanes (described, for example, in Chem. Pharm. Bull. 1990, 38, 2719-2725; J. Org. Chem. 1962, 27, 1647-1650). The halogen-substituted amino compounds of the formula (II) are either commercially available or can be prepared by literature procedures. One method for preparing suitable halogen-substituted amino compounds (II) is, for example, the reduction of corresponding carboximides (described, for example, in EP30092) or corresponding oximes or azides (described, for example, in Chem. Ber. 1988, 119, 2233) or nitro compounds (described, for example, in J. Am. Chem. Soc, 1953, 75, 5006). A further alternative consists in the treatment of corresponding aminocarboxylic acids with SF₄ in HF (described, for example, in J. Org. Chem. 1962, 27, 1406). The ring-opening of substituted aziridines with HF as described in J. Org. Chem. 1981, 46, 4938. Further methods for preparing halogen-substituted amino compounds (II) comprise the cleavage of corresponding phthalimides according to Gabriel (described, for example, in DE 3429048), the aminolysis of suitable haloalkyl halides (described, for example, in U.S. Pat. No. 2,539,406) or the degradation of corresponding carboxylic acid azides (described, for example, in DE3611195). Using suitable fluorinating agents (for example DAST), aminoaldehydes or -ketones can be converted into the corresponding difluoroalkylamines (WO2008008022), whereas amino alcohols form the corresponding monofluoroalkylamines (for example WO2006029115). Analogously, using suitable chlorinating and brominating agents, chloro- and bromoalkylamines, respectively, can be obtained from amino alcohols (J. Org. Chem. 2005, 70, 7364, or Org. Lett., 2004, 6, 1935).

Suitable substituted 2,4-dihalopyrimidines (III) are either commercially available or can be prepared according to literature procedures, for example from commercially available substituted uracils (for example R⁸═CN: J. Org. Chem. 1962, 27, 2264; J. Chem. Soc. 1955, 1834; Chem. Ber. 1909, 42, 734; R⁸═CF₃: J. Fluorine Chem. 1996, 77, 93; see also WO 2000/047539).

Using a suitable base at a temperature of from −30° C. to +80° C. in a suitable solvent, such as, for example, dioxane, THF, dimethylformamide or acetonitrile, initially an amine (II) is reacted with a 2,4-dihalopyrimidine (III) over a period of 1-24 h. Suitable for the use as base are, for example, inorganic salts, such as NaHCO₃, Na₂CO₃ or K₂CO₃, organometallic compounds, such as LDA or NaHMDS, or amine bases, such as ethyldiisopropylamine, DBU, DBN or tri-n-butylamine. Alternatively, the reaction can also be carried out as described, for example, in Org. Lett. 2006, 8, 395 with the aid of a suitable transition metal catalyst, such as, for example, palladium, together with a suitable ligand, such as, for example, triphenylphosphine or xanthphos.

One way of preparing the compound (VI) is shown in Scheme 2.

The intermediate (V) is reacted in the presence of Brønsted acids, such as, for example, anhydrous hydrochloric acid, camphorsulphonic acid or p-toluenesulphonic acid, in a suitable solvent, such as, for example, dioxane, THF, DMSO, DME, 2-methoxyethanol, n-butanol or acetonitrile, at a temperature of 0° C.-140° C. over a period of 1-48 h with an aromatic amine (IV). Analogously described, for example, in Bioorg. Med. Chem. Lett. 2006, 16, 2689; GB2002 A1-2369359, Org. Lett. 2005, 7, 4113.

Alternatively, the reaction of (V) and (IV) to give (VI) can also be carried out with base catalysis, i.e. using, for example, carbonates, such as potassium carbonate, alkoxides, such as potassium tert-butoxide, or hydrides, such as sodium hydride, where the catalytic use of a transition metal, such as, for example, palladium, together with a suitable ligand, such as, for example, xanthphos, may also be useful.

Finally, it is possible to carry out the reaction of (V) and (IV) to give (VI) in the absence of solvents and/or Brønsted acids (described, for example, in Bioorg. Med. Chem. Lett. 2006, 16, 108; Bioorg. Med. Chem. Lett. 2005, 15, 3881).

The substituted aromatic amines (IV) are either commercially available or they can be prepared by methods known from the literature from commercially available precursors (also see next section under (VII)).

A further way of preparing the compound (VI) is shown in Scheme 3.

The substituted aromatic amines (VII) are either commercially available or can be prepared from commercially available precursors by methods known from the literature. Aromatic amines carrying one or more identical or different substituents in the aromatic moiety can be prepared by a large number of methods described in the relevant literature. By way of example, some of the methods are mentioned below. (Scheme 8)

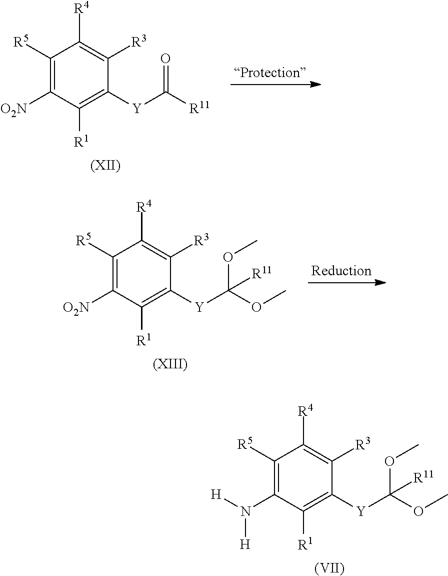

Nitroaromatic carbonyl compounds (XII) can be converted by standard methods into the corresponding dimethoxyacetals (XIII). The resulting compounds can then be reduced by methods known from the literature to give the corresponding amino compounds (VII). (for example Org. Biomol. Chem. 2006, 4, 3778; Org. Synth. 1949, 29, 6; Tetrahedron Lett. 2007, 48, 4727.)

Intermediates of the formula (V) can be reacted in the presence of Brønsted acids, such as, for example, hydrochloric acid, camphorsulphonic acid or 4-toluenesulphonic acid, in a suitable solvent, such as, for example, dioxane, THF, DMSO, DME, 2-methoxyethanol, n-butanol or acetonitrile, at a temperature of 0° C.-140° C. for 1-48 hours with the aromatic amine (VII) to give the free carbonyl compounds (VI).

Finally, it is possible to carry out the reaction of (V) and (VII) to give (VI) in the absence of solvents.

One way of preparing diaminopyrimidines of the formula (I) in which R² or R³ represents a group of the formula E1 [(Ia)] is shown in Scheme 4.

Diol compounds of the formula (VIII) are either commercially available or can be prepared by literature procedures, such as, for example, by dihydroxylation of double bonds, hydrolysis of epoxides, reduction of dicarbonyls, reductive coupling of carbonyl compounds (see, for example, Chem. Rev 1994, 94, 2483; Tetrahedron 2006, 62, 12137; Synthesis 2006, 557; Eur. J. Org. Chem. 1998, 2839; Org. Biomol. Chem. 2004, 2, 2403; Synthesis 2008, 1641; Synth. Commun. 2008, 38, 232; Tetrahedron Lett. 2006, 47, 3659; Angew. Chem. Int. Ed. 1999, 38, 3026; J. Am. Chem. Soc. 1990, 112, 6447).

By reaction with diols of the formula (VIII), carbonyl compounds of the formula (VI) can be converted into acetals or ketals of the formula (Ia). This reaction is carried out in the presence of Brønsted acids, such as, for example, anhydrous hydrochloric acid, sulphuric acid, camphorsulphonic acid or 4-toluenesulphonic acid, or Lewis acids, such as, for example, BF₃.Et₂O, in a suitable solvent, such as, for example, dioxane, THF, benzene, toluene or cyclohexane, at a temperature of −20° C.-140° C. for 1-48 hours.

Acetalization/ketalization is a known synthesis method. It is carried out with the aid of an alcohol, if appropriate in the presence of a solvent, and can be carried out in a wide temperature range or else under the effect of microwave radiation. In general, it is catalysed by Brønsted or Lewis acids, if appropriate in the presence of dehydrating agents (for a review, see: Greene's Protective Groups in Organic Synthesis 4^{th} Ed., 2007, pp. 431-532; Synthesis 1981, 501; specific examples in: Tetrahedron Lett. 2008, 64, 3287; Tetrahedron Lett. 2006, 47, 9317; Letters in Org. Chem. 2005, 2, 151; Adv. Synth. Catal. 2004, 346, 446 and the literature references cited therein). The reaction can also be carried out, for example, in the presence of NBS (for example: Synthesis 2005, 279) and, in rare cases, also under basic conditions (for example: Org. Lett. 2006, 8, 3745).

However, for acetalizations/ketalizations, alcohols are not necessarily required as reactants; they can also be prepared from oxiranes, carbonates and other compounds (for example: Synthesis 1981, 501).

A further way of preparing the compounds of the formula (Ia) is shown in Scheme 5.

The substituted aromatic amines (IX) are either commercially available or can be prepared by methods known from the literature from commercially available precursors. Aromatic amines carrying one or more identical or different substituents in the aromatic moiety can be prepared by a large number of methods described in the relevant literature. By way of example, some of the methods are mentioned below (Scheme 9).

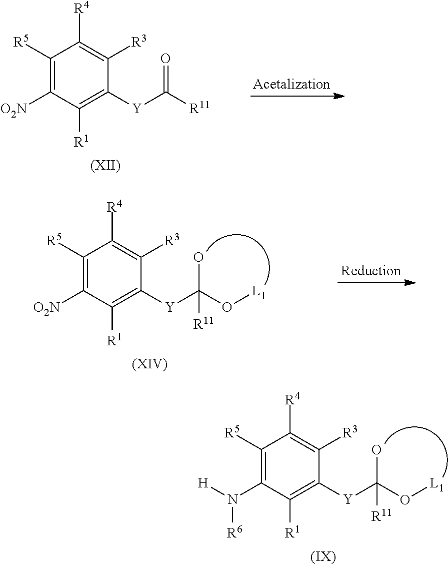

Nitroaromatic carbonyl compounds (XII) can be converted by standard methods into the corresponding cyclic acetals/ketals (XIV). By methods known from the literature, the resulting compounds can then be converted into the corresponding amino compounds (IX) (see literature references, Scheme xy).

Finally, the intermediate (V) is reacted in the presence of Brønsted acids, such as, for example, anhydrous hydrochloric acid, camphorsulphonic acid or p-toluenesulphonic acid, in a suitable solvent, such as, for example, dioxane, THF, DMSO, DME, 2-methoxyethanol, n-butanol or acetonitrile, at a temperature of 0° C.-140° C. over a period of 1-48 h with an aromatic amine (IX). The compound (Ia) according to the invention is formed in the process.

Alternatively, the reaction of (V) and (IX) to give (Ia) can also be carried out with base catalysis, i.e. using, for example, carbonates, such as potassium carbonate, alkoxides, such as potassium tert-butoxide, or hydrides, such as sodium hydride, where the catalytic use of a transition metal such as, for example, palladium, together with a suitable ligand, such as, for example, xanthphos, may also be useful.

Finally, it is possible to carry out the reaction of (V) and (IX) to give (Ia) in the absence of solvents and/or Brønsted acids (described, for example, in Bioorg. Med. Chem. Lett. 2006, 16, 108; Bioorg. Med. Chem. Lett. 2005, 15, 3881).

One way of preparing the compounds of the formula (I) in which R² or R³ represents a group of the formula E2, (Ib), is shown in Scheme 6.

Compounds of the formula (X) are either commercially available or can be prepared by numerous literature procedures (see, for example: J. Org. Chem. 2007, 72, 3776; Chem. Pharm. Bull. 2006, 54, 141; Tetrahedron Asymmetry 2004, 15, 895; J. Chem. Soc., Perkin Trans. 1 2002, 2282; Acta Chem. Scand. 1996, 50, 158; J. Am. Chem. Soc. 1985, 107, 4175; J. Org. Chem. 1967, 32, 282).

Carbonyl compounds of the formula (VI) are reacted with compounds of the formula (X) to give dithio or monothio acetals of the formula (Ib). This reaction can be carried out in the presence of Brønsted acids, such as, for example, anhydrous hydrochloric acid, sulphuric acid, camphorsulphonic acid or p-toluenesulphonic acid, or of Lewis acids, such as, for example, BF₃.Et₂O, in a suitable solvent, such as, for example, dioxane, THF, dichloromethane, benzene, toluene or cyclohexane, at a temperature of −20° C.-140° C. over a period of 1-48 h.

In the literature, there are numerous descriptions of the synthesis of cyclic dithio or monothio acetals and ketals. The synthesis is carried out with the aid of dithiols or hydroxythiols, if appropriate in the presence of a solvent, and can be carried out over a wide temperature range or else under the effect of microwave radiation. In general, the reaction is catalysed by Brønsted or Lewis acids, if appropriate in the presence of dehydrating agents. For a review, see: Greene's Protective Groups in Organic Synthesis 4^{th} Ed., 2007, pp. 431-532; for specific examples, see Tetrahedron Lett. 2008, 49, 1919; Synthesis 2006, 2761; Tetrahedron Lett. 2003, 44, 8597; Synlett 1999, 415 and the literature references cited therein).

One way of preparing the compounds of the formula (I) in which R² or R³ represents a group of the formula E3, (Ic), is shown in Scheme 7.

Carbonyl compounds of the formula (VI) are reacted with compounds of the formula (XI) to give compounds of the formula (Ic). This reaction can be carried out in the presence of Brønsted acids, such as, for example, anhydrous hydrochloric acid, sulphuric acid, camphorsulphonic acid or p-toluenesulphonic acid, or of Lewis acids, such as, for example, BF₃.Et₂O, in a suitable solvent, such as, for example, dioxane, THF, dichloromethane, benzene, toluene or cyclohexane, at a temperature of −20° C.-140° C. over a period of 1-48 h.

Numerous of these reactions have been described in the literature, and the reaction conditions are similar to the conditions described above (literature references, see Schemes 4 and 6).

Another way of preparing diaminopyrimidines of the formula (I) in which R² or R³ represents a group of the formula E2, (Ib), is shown in Scheme 10.

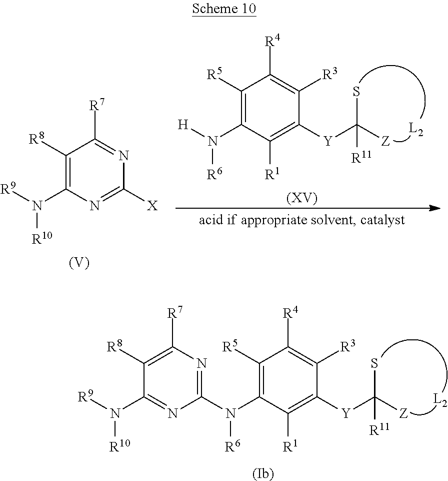

Another way of preparing diaminopyrimidines of the formula (I) in which R² or R³ represents a group of the formula E3, (Ic), is shown in Scheme 11.

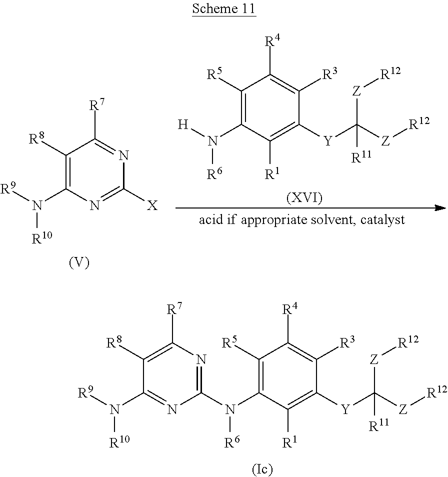

In general, it also possible to choose another route for preparing the compounds (Ia), (Ib), (Ic), (VI) according to the invention, such as shown in Scheme 12:

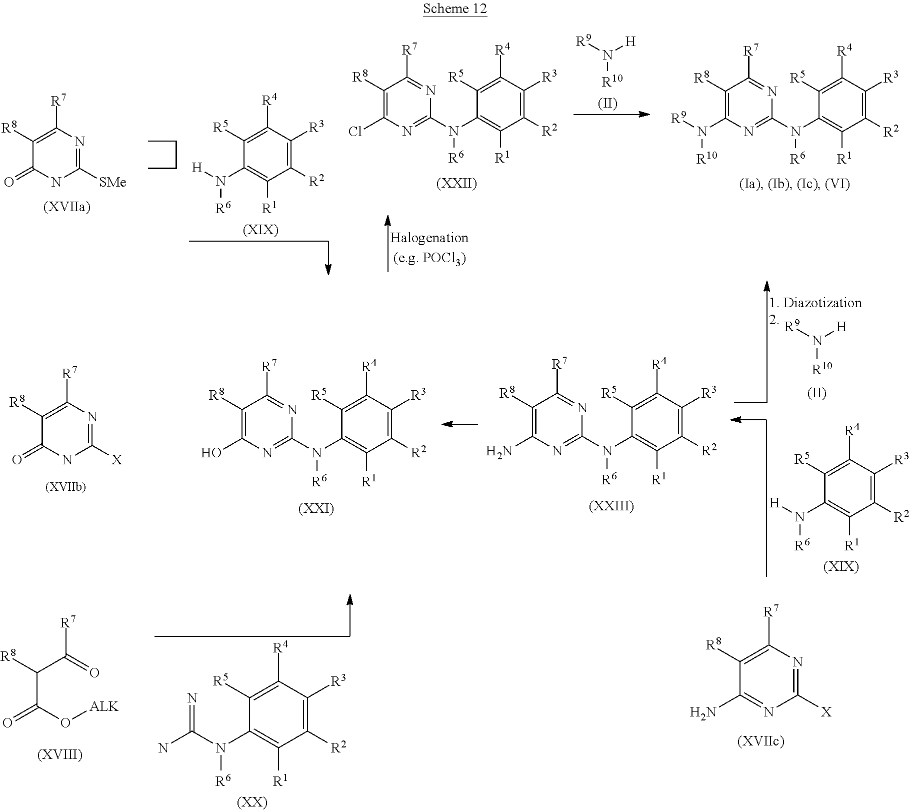

Compounds of the formula (VI) can be converted into the compounds (Ia), (Ib) and (Ic) according to the invention by using the above-described processes (see Schemes 4, 6 and 7).

In general, compounds of the formula (I) can be prepared, for example, by sequential nucleophilic addition of an aliphatic amine (II) and an aromatic amine (XIX) to a suitable substituted pyrimidine (III), as outlined below in Scheme 13:

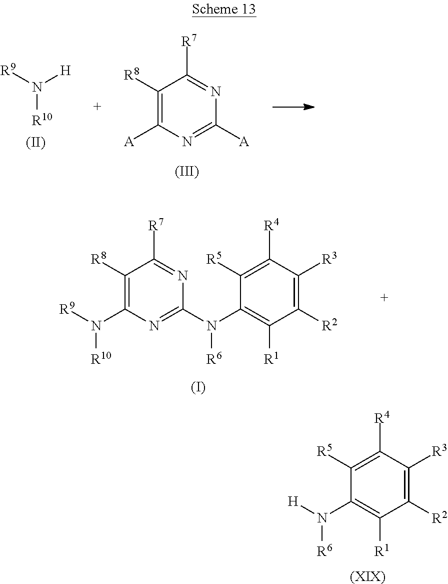

Here, A, in each case independently of one another, represent suitable leaving groups, for example a halogen atom (F, Cl, Br, I), SMe, SO₂Me, SOMe or else triflate (CF₃SO₂O: for pyrimidines known from WO 05/095386).

The synthesis of diaminopyrimidines of the formula (I) according to Scheme 13 or else by other routes has been described in the literature in many different instances (see, for example, also WO 07/140,957, WO 06/021544, WO 07/072,158, WO 07/003,596, WO 05/016893, WO 05/013996, WO 04/056807, WO 04/014382, WO 03/030909).

The processes according to the invention for preparing the compounds of the formulae (I), (Ia), (Ib) and (Ic) are preferably carried out using one or more reaction auxiliaries.

Suitable reaction auxiliaries are, if appropriate, the customary inorganic or organic bases or acid acceptors. These preferably include alkali metal and alkaline earth metal acetates, amides, carbonates, bicarbonates, hydrides, hydroxides, and alkoxides, such as, for example, sodium acetate, potassium acetate or calcium acetate, lithium amide, sodium amide, potassium amide or calcium amide, sodium carbonate, potassium carbonate or calcium carbonate, sodium bicarbonate, potassium bicarbonate, or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, i-, s- or t-butoxide or potassium methoxide, ethoxide, n- or i-propoxide, n-, i-, s- or t-butoxide; furthermore also basic organic nitrogen compounds, such as, for example, trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]-octane (DABCO), 1,5-diazabicyclo[4.3.0]non-5-ene (DBN) or 1,8-diazabicyclo[5.4.0]undec-7-ene (DBU).

The processes according to the invention are preferably carried out using one or more diluents. Suitable diluents are virtually all inert organic solvents. These preferably include aliphatic and aromatic, unbranched or halogenated hydrocarbons, such as pentane, hexane, heptane, cyclohexane, petroleum ether, benzine, ligroin, benzene, toluene, xylene, methylene chloride, ethylene chloride, chloroform, carbon tetrachloride, chlorobenzene and o-dichlorobenzene, ethers, such as diethyl ether and dibutyl ether, glycol dimethyl ether and diglycol dimethyl ether, tetrahydrofuran and dioxane, ketones, such as acetone, methyl ethyl ketone, methyl isopropyl ketone or methyl isobutyl ketone, esters, such as methyl acetate or ethyl acetate, nitriles, such as, for example, acetonitrile or propionitrile, amides, such as, for example, dimethylformamide, dimethylacetamide and N-methylpyrrolidone, and also dimethyl sulphoxide, tetramethylene sulphone and hexamethylphosphoric triamide and DMPU.

In the processes according to the invention, the reaction temperatures can be varied within a relatively wide range. In general, the processes are carried out at temperatures between 0° C. and 250° C., preferably at temperatures between 10° C. and 185° C.

The processes according to the invention are generally carried out under atmospheric pressure. However, it is also possible to operate under elevated or reduced pressure.

For carrying out the processes according to the invention, the starting materials required in each case are generally employed in approximately equimolar amounts. However, it is also possible to use in each case one of the components employed in a relatively large excess. Work-up in the processes according to the invention is in each case carried out by customary methods (cf. the Preparation Examples).

Some of the compounds of the formula (V) are novel and thus also form part of the subject-matter of the present invention.

Novel are compounds of the formula (V)

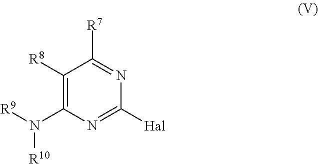

in which
R⁷ represents hydrogen,
   and, if
R⁸ represents CF₃, CFH₂ or CF₂H,
Hal represents F, Cl, Br or I,
R⁹ represents hydrogen, ethyl, propyl, propan-2-yl, 2-methoxyethan-1-yl, prop-2-en-1-yl, CH₂OCH₃, COMe, COOMe, COOEt, COOtertBu, COCF₃ or benzyl,
R¹⁰ represents ethyl, propyl, cyclopropyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, 2-methylcyclobut-1-yl, 3-methylcyclobut-1-yl, butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, butan-2-yl, 2,2,2-trifluoroethyl, 2,2-difluoroethyl, 2-methoxyethan-1-yl, 2-methylmercaptoethan-1-yl, 2-fluoroethan-1-yl, 2-chloroethan-1-yl, 2-cyanoethan-1-yl, 1-methoxypropan-2-yl, 3-methoxypropan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1-yl or 1-fluoropropan-2-yl,
   or
R⁹ and R¹⁰ together with the nitrogen atom to which they are attached form an unsubstituted or substituted 3- to 7-membered saturated cycle which may contain up to one further heteroatom,
   where the substituents independently of one another are selected from the group consisting of methyl, fluorine, chlorine and/or bromine atoms, cyano, hydroxyl, methoxy or CF₃,
   and where the heteroatoms are selected from the group consisting of oxygen, sulphur and nitrogen.

Novel are compounds of the formula (V)

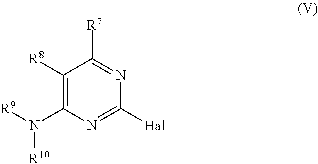

in which
R⁷ represents hydrogen,
   and, if
R⁸ represents iodine,
Hal represents F, Cl, Br or I,
R⁹ represents hydrogen, methyl, ethyl, propyl, propan-2-yl, 2-methoxyethan-1-yl, prop-2-en-1-yl, CH₂OCH₃, COMe, COOMe, COOEt, COOtertBu, COCF₃ or benzyl,
R¹⁰ represents methyl, ethyl, propyl, cyclopropyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, 2-methylcyclobut-1-yl, 3-methylcyclobut-1-yl, butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, butan-2-yl, 2,2,2-trifluoroethyl, 2,2-difluoroethyl, 2-methoxyethan-1-yl, 2-methylmercaptoethan-1-yl, 2-fluoroethan-1-yl, 2-chloroethan-1-yl, 2-cyanoethan-1-yl, 1-methoxypropan-2-yl, 3-methoxypropan-1-yl, 1-methyl-mercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1-yl or 1-fluoropropan-2-yl,
   or
R⁹ and R¹⁰ together with the nitrogen atom to which they are attached form an unsubstituted or substituted 3- to 7-membered saturated cycle which may contain up to one further heteroatom,
   where the substituents independently of one another are selected from the group consisting of methyl, fluorine, chlorine and/or bromine atoms, cyano, hydroxyl, methoxy or CF₃,
   and where the heteroatoms are selected from the group consisting of oxygen, sulphur and nitrogen.

Novel are compounds of the formula (V)

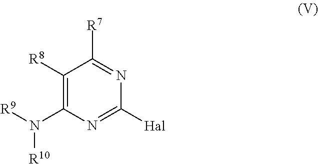

in which
R⁷ represents hydrogen,
   and, if
R⁸ represents SMe, SOMe or SO₂Me,
Hal represents F, Cl, Br or I,
R⁹ represents hydrogen, methyl, ethyl, propyl, propan-2-yl, 2-methoxyethan-1-yl, prop-2-en-1-yl, CH₂OCH₃, COMe, COOMe, COOEt, COOtertBu, COCF₃ or benzyl,
R¹⁰ represents ethyl, propyl, cyclopropyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, 2-methylcyclobut-1-yl, 3-methylcyclobut-1-yl, butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, butan-2-yl, 2,2,2-trifluoroethyl, 2,2-difluoroethyl, 2-methoxyethan-1-yl, 2-methylmercaptoethan-1-yl, 2-fluoroethan-1-yl, 2-chloroethan-1-yl, 2-cyanoethan-1-yl, 1-methoxypropan-2-yl, 3-methoxypropan-1-yl, 1-methyl-mercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1-yl or 1-fluoropropan-2-yl,
   or
R⁹ and R¹⁰ together with the nitrogen atom to which they are attached form an unsubstituted or substituted 3- to 7-membered saturated cycle which may contain up to one further heteroatom,
   where the substituents independently of one another are selected from the group consisting of methyl, fluorine, chlorine and/or bromine atoms, cyano, hydroxyl, methoxy or CF₃,
   and where the heteroatoms are selected from the group consisting of oxygen, sulphur and nitrogen.

Novel are compounds of the formula (V)

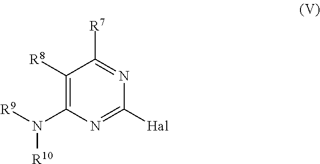

in which
R⁷ represents hydrogen,
   and, if
R⁸ represents cyano,
Hal represents F, Cl, Br or I,
R⁹ represents hydrogen, methyl, propyl, propan-2-yl, 2-methoxyethan-1-yl, prop-2-en-1-yl, CH₂OCH₃, COMe, COOMe, COOEt, COOtertBu, COCF₃ or benzyl,
R¹⁰ represents propyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, 2-methylcyclobut-1-yl, 3-methylcyclobut-1-yl, 3-methylbut-1-yl, 2-methylbut-1-yl, 1-fluoroprop-2-yl, cyclopentyl, propan-2-yl, pentan-3-yl, pentan-2-yl, pentyl, butan-2-yl, 2,2-difluoroethyl, 2-methylmercaptoethan-1-yl, 2-chloroethan-1-yl, 1-methoxypropan-2-yl, 3-methoxy-propan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1-yl or 1-fluoropropan-2-yl.

Some of the compounds of the formula (VI) are novel and thus also form part of the subject-matter of the present invention.

Compounds of the formula (VI) according to the invention and their agrochemically active salts are highly suitable for controlling phytopathogenic harmful fungi. The compounds according to the invention mentioned above have in particular strong fungicidal activity and can be used in crop protection, in the domestic and hygiene field and in the protection of materials.

Novel are compounds of the formula (VI)

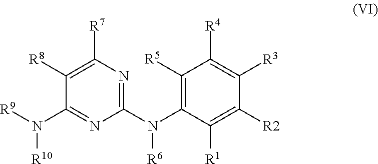

in which
R¹ and R⁵ represent hydrogen,
R² to R⁴ independently of one another represent hydrogen, OH, halogen, cyano, C₁-C₄-alkyl, C₁-C₄-alkoxy, C₁-C₄-haloalkyl, C₁-C₄-alkoxycarbonyl, NMe₂, SCF₃, SCH₃, OCF₂H or OCF₃,
   where exactly one of the radicals R² and R³ represents a group of the formula E4

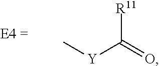

in which the symbols have the meanings below:
R¹¹ represents hydrogen, CH₂OH, optionally branched C₁-C₄-alkyl, optionally branched C₁-C₄-alkoxy-C₁-C₄-alkyl, optionally branched C₁-C₄-haloalkyl, C₁-C₄-alkoxycarbonylmethyl, optionally branched C₃-C₆-cycloalkyl, CH₂CN, CH₂CH₂CN, 3-oxobutanoyl, thiophen-2-yl, CH₂CH₂OH, phenyl or CH₂Ph,
R¹⁰ represents methyl, ethyl, propyl, cyclopropyl, 1-cyclopropyleth-1-yl, 2-methylcyclopropyl, 2,2-dimethylcyclopropyl, 2,2-dimethylprop-1-yl, tert-butyl, cyclobutyl, 2-methylcyclobut-1-yl, 3-methylcyclobut-1-yl, butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, butan-2-yl, 2,2,2-trifluoroethyl, 2,2-difluoroethyl, 2-methoxyethan-1-yl, 2-methyl-mercaptoethan-1-yl, 2-fluoroethan-1-yl, 2-chloroethan-1-yl, 2-cyanoethan-1-yl, 1-methoxypropan-2-yl, 3-methoxypropan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-penta-fluoropropyl, 1,1,1-trifluoropropan-1-yl, 1,1,1-trifluorobutan-2-yl, 1,1,1-trifluorobutan-3-yl, 2-methylprop-2-en-1-yl or 1-fluoropropan-2-yl,
   or
R⁹ and R¹⁰ together with the nitrogen atom to which they are attached form an optionally branched, optionally substituted 3- to 7-membered saturated cycle which may contain up to one further heteroatom,
   where the substituents independently of one another are selected from the group consisting of methyl, fluorine, chlorine and/or bromine atoms, cyano, hydroxyl, methoxy and CF₃,
   and
Y, R⁶, R⁷, R⁸ and R⁹ have the general, preferred, particularly preferred and very particularly preferred meanings as defined above,
   and agrochemically active salts thereof.

The invention furthermore provides the non-medicinal use of the diaminopyrimidines according to the invention for controlling unwanted microorganisms.

The invention furthermore provides a composition for controlling unwanted microorganisms, comprising at least one diaminopyrimidine according to the present invention.

Moreover, the invention relates to a method for controlling unwanted microorganisms, characterized in that the diaminopyrimidines according to the invention are applied to the microorganisms and/or their habitat.

The compounds according to the invention have strong microbicidal action and can be used for controlling unwanted microorganisms, such as fungi and bacteria, in crop protection and in the protection of materials.

The diaminopyrimidines of the formula (I) according to the invention have very good fungicidal properties and can be used in crop protection, for example for controlling Plasmodiophoromycetes, Oomycetes, Chytridiomycetes, Zygomycetes, Ascomycetes, Basidiomycetes and Deuteromycetes.

In crop protection, bactericides can be used for controlling Pseudomonadaceae, Rhizobiaceae, Enterobacteriaceae, Corynebacteriaceae and Streptomycetaceae.

The fungicidal compositions according to the invention can be used for the curative or protective control of phytopathogenic fungi. Accordingly, the invention also relates to curative and protective methods for controlling phytopathogenic fungi using the active compounds or compositions according to the invention, which are applied to the seed, the plant or plant parts, the fruit or the soil on which the plants grow.

The compositions according to the invention for controlling phytopathogenic fungi in crop protection comprise an effective, but non-phytotoxic amount of the active compounds according to the invention. “Effective, but non-phytotoxic amount” means an amount of the composition according to the invention which is sufficient to control the fungal disease of the plant in a satisfactory manner or to eradicate the fungal disease completely, and which, at the same time, does not cause any significant symptoms of phytotoxicity. In general, this application rate may vary within a relatively wide range. It depends on a plurality of factors, for example on the fungus to be controlled, the plant, the climatic conditions and the ingredients of the compositions according to the invention.

According to the invention, it is possible to treat all plants and parts of plants. Plants are to be understood here as meaning all plants and plant populations, such as wanted and unwanted wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by varietal property rights. Parts of plants are to be understood as meaning all above-ground and below-ground parts and organs of the plants, such as shoot, leaf, flower and root, examples which may be mentioned being leaves, needles, stems, trunks, flowers, fruit bodies, fruits and seeds and also roots, tubers and rhizomes. Plant parts also include harvested material and vegetative and generative propagation material, for example seedlings, tubers, rhizomes, cuttings and seeds.

The following plants may be mentioned as plants which can be treated according to the invention: cotton, flax, grapevines, fruit, vegetables, such as Rosaceae sp. (for example pomaceous fruit, such as apples and pears, but also stone fruit, such as apricots, cherries, almonds and peaches and soft fruit such as strawberries), Ribesioidae sp., Juglandaceae sp., Betulaceae sp., Anacardiaceae sp., Fagaceae sp., Moraceae sp., Oleaceae sp., Actimidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit), Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumbers), Alliaceae sp. (for example leek, onions), Papilionaceae sp. (for example peas); major crop plants, such Gramineae sp. (for example maize, lawn, cereals such as wheat, rye, rice, barley, oats, millet and triticale), Asteraceae sp. (for example sunflowers), Brassicaceae sp. (for example white cabbage, red cabbage, broccoli, cauliflowers, brussel sprouts, pak choi, kohlrabi, garden radish, and also oilseed rape, mustard, horseradish and cress), Fabacae sp. (for example beans, peas), Papilionaceae sp. (for example soya beans), Solanaceae sp. (for example potatoes), Chenopodiaceae sp. (for example sugarbeet, fodderbeet, swiss chard, beetroot); crop plants and ornamental plants in garden and forest; and also in each case genetically modified varieties of these plants. Preferably, cereal plants are treated according to the invention.

Some pathogens of fungal diseases which can be treated according to the invention may be mentioned by way of example, but not by way of limitation:

Diseases caused by powdery mildew pathogens, such as, for example, Blumeria species, such as, for example, Blumeria graminis; Podosphaera species, such as, for example, Podosphaera leuco-tricha; Sphaerotheca species, such as, for example, Sphaerotheca fuliginea; Uncinula species, such as, for example, Uncinula necator;
Diseases caused by rust disease pathogens, such as, for example, Gymnosporangium species, such as, for example, Gymnosporangium sabinae; Hemileia species, such as, for example, Hemileia vastatrix; Phakopsora species, such as, for example, Phakopsora pachyrhizi and Phakopsora meibomiae; Puccinia species, such as, for example, Puccinia recondita or Puccinia triticina; Uromyces species, such as, for example, Uromyces appendiculatus;
Diseases caused by pathogens from the group of the Oomycetes, such as, for example, Bremia species, such as, for example, Bremia lactucae; Peronospora species, such as, for example, Peronospora pisi or P. brassicae; Phytophthora species, such as, for example Phytophthora infestans; Plasmopara species, such as, for example, Plasmopara viticola; Pseudoperonospora species, such as, for example, Pseudoperonospora humuli or Pseudoperonospora cubensis; Pythium species, such as, for example, Pythium ultimum;
Leaf blotch diseases and leaf wilt diseases caused, for example, by Alternaria species, such as, for example, Alternaria solani; Cercospora species, such as, for example, Cercospora beticola; Cladiosporium species, such as, for example, Cladiosporium cucumerinum; Cochliobolus species, such as, for example, Cochliobolus sativus (conidia form: Drechslera, Syn: Helminthosporium); Colletotrichum species, such as, for example, Colletotrichum lindemuthanium; Cycloconium species, such as, for example, Cycloconium oleaginum; Diaporthe species, such as, for example, Diaporthe citri; Elsinoe species, such as, for example, Elsinoe fawcettii; Gloeosporium species, such as, for example, Gloeosporium laeticolor; Glomerella species, such as, for example, Glomerella cingulata; Guignardia species, such as, for example, Guignardia bidwelli; Leptosphaeria species, such as, for example, Leptosphaeria maculans; Magnaporthe species, such as, for example, Magnaporthe grisea; Microdochium species, such as, for example, Microdochium nivale; Mycosphaerella species, such as, for example, Mycosphaerella graminicola and M. fijiensis; Phaeosphaeria species, such as, for example, Phaeosphaeria nodorum; Pyrenophora species, such as, for example, Pyrenophora teres; Ramularia species, such as, for example, Ramularia collo-cygni; Rhynchosporium species, such as, for example, Rhynchosporium secalis; Septoria species, such as, for example, Septoria apii; Typhula species, such as, for example, Typhula incarnata; Venturia species, such as, for example, Venturia inaequalis;
Root and stem diseases caused, for example, by Corticium species, such as, for example, Corticium graminearum; Fusarium species, such as, for example, Fusarium oxysporum; Gaeumannomyces species, such as, for example, Gaeumannomyces graminis; Rhizoctonia species, such as, for example Rhizoctonia solani; Tapesia species, such as, for example, Tapesia acuformis; Thielaviopsis species, such as, for example, Thielaviopsis basicola;
Ear and panicle diseases (including maize cobs) caused, for example, by Alternaria species, such as, for example, Alternaria spp.; Aspergillus species, such as, for example, Aspergillus flavus; Cladosporium species, such as, for example, Cladosporium cladosporioides; Claviceps species, such as, for example, Claviceps purpurea; Fusarium species, such as, for example, Fusarium culmorum; Gibberella species, such as, for example, Gibberella zeae; Monographella species, such as, for example, Monographella nivalis; Septoria species, such as for example, Septoria nodorum;
Diseases caused by smut fungi, such as, for example, Sphacelotheca species, such as, for example, Sphacelotheca reiliana; Tilletia species, such as, for example, Tilletia caries; T. controversa; Urocystis species, such as, for example, Urocystis occulta; Ustilago species, such as, for example, Ustilago nuda; U. nuda tritici;
Fruit rot caused, for example, by Aspergillus species, such as, for example, Aspergillus flavus; Botrytis species, such as, for example, Botrytis cinerea; Penicillium species, such as, for example, Penicillium expansum and P. purpurogenum; Sclerotinia species, such as, for example, Sclerotinia sclerotiorum;
Verticilium species, such as, for example, Verticilium alboatrum;
Seed- and soil-borne rot and wilt diseases, and also diseases of seedlings, caused, for example, by Fusarium species, such as, for example, Fusarium culmorum; Phytophthora species, such as, for example, Phytophthora cactorum; Pythium species, such as, for example, Pythium ultimum; Rhizoctonia species, such as, for example, Rhizoctonia solani; Sclerotium species, such as, for example, Sclerotium rolfsii;
Cancerous diseases, galls and witches' broom caused, for example, by Nectria species, such as, for example, Nectria galligena;
Wilt diseases caused, for example, by Monilinia species, such as, for example, Monilinia laxa;
Deformations of leaves, flowers and fruits caused, for example, by Taphrina species, such as, for example, Taphrina deformans;
Degenerative diseases of woody plants caused, for example, by Esca species, such as, for example, Phaemoniella clamydospora and Phaeoacremonium aleophilum and Fomitiporia mediterranea;
Diseases of flowers and seeds caused, for example, by Botrytis species, such as, for example, Botrytis cinerea;
Diseases of plant tubers caused, for example, by Rhizoctonia species, such as, for example, Rhizoctonia solani; Helminthosporium species, such as, for example, Helminthosporium solani;
Diseases caused by bacterial pathogens, such as, for example, Xanthomonas species, such as, for example, Xanthomonas campestris pv. oryzae; Pseudomonas species, such as, for example, Pseudomonas syringae pv. lachrymans; Erwinia species, such as, for example, Erwinia amylovora.

Preference is given to controlling the following diseases of soya beans:

Fungal diseases on leaves, stems, pods and seeds caused, for example, by alternaria leaf spot (Alternaria spec. atrans tenuissima), anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

In the present case, undesired microorganisms are understood as meaning phytopathogenic fungi and bacteria. Thus, the substances according to the invention can be employed for protecting plants against attack by the abovementioned pathogens within a certain period of time after the treatment. The period of time within which their protection is effected is generally extended from 1 to 10 days, preferably 1 to 7 days, after the plants have been treated with the active compounds.

The fact that the active compounds, at the concentrations required for the controlling of plant diseases, are well tolerated by plants permits the treatment of above-ground plant parts, of vegetative propagation material and seed, and of the soil.

In this context, the active compounds according to the invention can be employed particularly successfully for controlling cereal diseases such as, for example, against Erysiphe species, against Puccinia and against Fusaria species, rice diseases such as, for example against Pyricularia and Rhizoctonia and diseases in viticulture, fruit production and vegetable production such as, for example, against Botrytis, Venturia, Sphaerotheca and Podosphaera species.

The active compounds according to the invention are also suitable for increasing the yield. Moreover, they display a low degree of toxicity and are well tolerated by plants.

If appropriate, the compounds according to the invention can, at certain concentrations or application rates, also be used as herbicides, safeners, growth regulators or agents to improve plant properties, or as microbicides, for example as fungicides, antimycotics, bactericides, viricides (including agents against viroids) or as agents against MLO (Mycoplasma-like organisms) and RLO (Rickettsia-like organisms). If appropriate, they can also be employed as insecticides. If appropriate, they can also be employed as intermediates or precursors for the synthesis of other active compounds.

If appropriate, at certain concentrations and application rates, the active compounds according to the invention can also be used as herbicides, for influencing plant growth. If appropriate, they can also be employed as intermediates and precursors for the synthesis of further active compounds.

The active compounds according to the invention, in combination with good plant tolerance and favourable toxicity to warm-blooded animals and being tolerated well by the environment, are suitable for protecting plants and plant organs, for increasing harvest yields and for improving the quality of harvested material in agriculture, in horticulture, in animal husbandry, in forests, in gardens and leisure facilities, in the protection of stored products and of materials, and in the hygiene sector. They are preferably employed as crop protection agents. They are active against normally sensitive and resistant species and against all or some stages of development.

The treatment according to the invention of the plants and plant parts with the active compounds or compositions is carried out directly or by action on their surroundings, habitat or storage space using customary treatment methods, for example by dipping, spraying, atomizing, irrigating, evaporating, dusting, fogging, broadcasting, foaming, painting, spreading-on, watering (drenching), drip irrigating and, in the case of propagation material, in particular in the case of seeds, furthermore as a powder for dry seed treatment, a solution for wet seed treatment, a water-soluble powder for slurry treatment, by encrusting, by coating with one or more coats, etc. It is furthermore possible to apply the active compounds by the ultra-low-volume method or to inject the active compound preparation or the active compound itself into the soil.

In addition, by the treatment according to the invention it is possible to reduce the mycotoxin content in the harvested material and the foodstuffs and feedstuffs prepared therefrom. Particular, but not exclusive, mention may be made here of the following mycotoxins: deoxynivalenol (DON), nivalenol, 15-Ac-DON, 3-Ac-DON, T2- and HT2-toxin, fumonisins, zearalenone, moniliformin, fusarin, diacetoxyscirpenol (DAS), beauvericin, enniatin, fusaroproliferin, fusarenol, ochratoxins, patulin, ergot alkaloids and aflatoxins produced, for example, by the following fungi: Fusarium spec., such as Fusarium acuminatum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides, inter alia, and also by Aspergillus spec., Penicillium spec., Claviceps purpurea, Stachybotrys spec., inter alia.

In the protection of materials, the compositions or active compounds according to the invention can furthermore be employed for protecting industrial materials against attack and destruction by unwanted microorganisms, such as, for example, fungi.

In the present context, industrial materials are understood as meaning nonliving materials which have been made for use in technology. For example, industrial materials which are to be protected by active compounds according to the invention from microbial modification or destruction can be glues, sizes, paper and board, textiles, leather, timber, paints and plastic articles, cooling lubricants and other materials which are capable of being attacked or destroyed by microorganisms. Parts of production plants, for example cooling-water circuits, which can be adversely affected by the multiplication of microorganisms may also be mentioned within the materials to be protected. Industrial materials which may be mentioned with preference for the purposes of the present invention are glues, sizes, paper and board, leather, timber, paints, cooling lubricants and heat-transfer fluids, especially preferably wood. The compositions or active compounds according to the invention can prevent disadvantageous effects such as rotting, decay, discoloration, decoloration or the formation of mould.

The method according to the invention for controlling unwanted fungi can also be employed for protecting storage goods. Here, storage goods are to be understood as meaning natural substances of vegetable or animal origin or process products thereof of natural origin, for which long-term protection is desired. Storage goods of vegetable origin, such as, for example, plants or plant parts, such as stems, leaves, tubers, seeds, fruits, grains, can be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The active compound combinations according to the invention can prevent disadvantageous effects, such as rotting, decay, discoloration, decoloration or the formation of mould.

Microorganisms capable of degrading or changing the industrial materials which may be mentioned are, for example, bacteria, fungi, yeasts, algae and slime organisms. The active compounds according to the invention preferably act against fungi, in particular moulds, wood-discoloring and wood-destroying fungi (Basidiomycetes) and against slime organisms and algae. Microorganisms of the following genera may be mentioned as examples: Alternaria, such as Alternaria tenuis; Aspergillus, such as Aspergillus niger; Chaetomium, such as Chaetomium globosum; Coniophora, such as Coniophora puetana; Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride; Escherichia, such as Escherichia coli; Pseudomonas, such as Pseudomonas aeruginosa; Staphylococcus, such as Staphylococcus aureus.

The present invention furthermore relates to a composition for controlling unwanted microorganisms comprising at least one of the diaminopyrimidines according to the invention. These are preferably fungicidal compositions comprising auxiliaries, solvents, carriers, surfactants or extenders suitable for use in agriculture.

According to the invention, a carrier is a natural or synthetic, organic or inorganic substance with which the active compounds are mixed or bonded for better applicability, in particular for application to plants or parts of plants or seed. The carrier, which may be solid or liquid, is generally inert and should be suitable for use in agriculture.

Suitable solid carriers are: for example ammonium salts and ground natural minerals, such as kaolins, clays, talc, chalk, quartz, attapulgite, montmorillonite or diatomaceous earth, and ground synthetic minerals, such as finely divided silica, alumina and silicates; suitable solid carriers for granules are: for example crushed and fractionated natural rocks, such as calcite, marble, pumice, sepiolite and dolomite, and also synthetic granules of inorganic and organic meals, and granules of organic material, such as paper, sawdust, coconut shells, maize cobs and tobacco stalks; suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are nonionic and/or ionic substances, for example from the classes of the alcohol/POE and/or POP ethers, acid and/or POP/POE esters, alkylaryl and/or POP/POE ethers, fat and/or POP/POE adducts, POE and/or POP polyols derivatives, POE and/or POP/sorbitan or sugar adducts, alkyl or aryl sulphates, sulphonates and phosphates, or the corresponding PO ether adducts. Furthermore suitable oligo- or polymers, for example those derived from vinylic monomers, from acrylic acid, from EO and/or PO alone or in combination with, for example, (poly)alcohols or (poly)amines. It is also possible to employ lignin and its sulphonic acid derivatives, unmodified and modified celluloses, aromatic and/or aliphatic sulphonic acids and their adducts with formaldehyde.

The active compounds can be converted to the customary formulations, such as solutions, emulsions, wettable powders, water- and oil-based suspensions, powders, dusts, pastes, soluble powders, soluble granules, granules for broadcasting, suspension-emulsion concentrates, natural materials impregnated with active compound, synthetic materials impregnated with active compound, fertilizers and also microencapsulations in polymeric substances.

The active compounds can be used as such, in the form of their formulations or the use forms prepared therefrom, such as ready-to-use solutions, emulsions, water- or oil-based suspensions, powders, wettable powders, pastes, soluble powders, dusts, soluble granules, granules for broadcasting, suspension-emulsion concentrates, natural materials impregnated with active compound, synthetic materials impregnated with active compound, fertilizers and also microencapsulations in polymeric substances. Application is carried out in a customary manner, for example by pouring, spraying, atomizing, broadcasting, dusting, foaming, painting-on, etc. It is furthermore possible to apply the active compounds by the ultra-low-volume method or to inject the preparation of active compound or the active compound itself into the soil. It is also possible to treat the seed of the plants.

The formulations mentioned can be prepared in a manner known per se, for example by mixing the active compounds with at least one customary extender, solvent or diluent, emulsifier, dispersant and/or binder or fixative, wetting agent, water repellant, if appropriate siccatives and UV stabilizers and if appropriate colorants and pigments, antifoams, preservatives, secondary thickeners, glues, gibberellins and other processing auxiliaries.

The compositions according to the invention include not only formulations which are already ready to use and can be applied to the plant or the seed using a suitable apparatus, but also commercial concentrates which have to be diluted with water prior to use.

The active compounds according to the invention can be present as such or in their (commercial) formulations and also in the use forms prepared from these formulations as a mixture with other (known) active compounds, such as insecticides, attractants, sterilants, bactericides, acaricides, nematicides, fungicides, growth regulators, herbicides, fertilizers, safeners and/or semiochemicals.

Suitable for use as auxiliaries are substances which are suitable for imparting to the composition itself and/or to preparations derived therefrom (for example spray liquors, seed dressings) particular properties such as certain technical properties and/or also particular biological properties. Typical suitable auxiliaries are: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

Liquefied gaseous extenders or carriers are liquids which are gaseous at ambient temperature and under atmospheric pressure, for example aerosol propellants, such as halogenated hydrocarbons, and also butane, propane, nitrogen and carbon dioxide.

Tackifiers, such as carboxymethylcellulose and natural and synthetic polymers in the form of powders, granules and latices, such as gum arabic, polyvinyl alcohol, polyvinyl acetate, or else natural phospholipids, such as cephalins and lecithins and synthetic phospholipids can be used in the formulations. Other possible additives are mineral and vegetable oils.

If the extender used is water, it is also possible to use, for example, organic solvents as auxiliary solvents. Suitable liquid solvents are essentially: aromatic compounds, such as xylene, toluene or alkylnaphthalenes, chlorinated aromatic compounds or chlorinated aliphatic hydrocarbons, such as chlorobenzenes, chloroethylenes or methylene chloride, aliphatic hydrocarbons, such as cyclohexane or paraffins, for example mineral oil fractions, alcohols, such as butanol or glycol, and also ethers and esters thereof, ketones, such as acetone, methyl ethyl ketone, methyl isobutyl ketone or cyclohexanone, strongly polar solvents, such as dimethylformamide and dimethyl sulphoxide, and also water.

The compositions according to the invention may additionally comprise further components, such as, for example, surfactants. Suitable surfactants are emulsifiers and/or foam-formers, dispersants or wetting agents having ionic or nonionic properties, or mixtures of these surfactants. Examples of these are salts of polyacrylic acid, salts of lignosulphonic acid, salts of phenolsulphonic acid or naphthalenesulphonic acid, polycondensates of ethylene oxide with fatty alcohols or with fatty acids or with fatty amines, substituted phenols (preferably alkylphenols or arylphenols), salts of sulphosuccinic esters, taurine derivatives (preferably alkyl taurates), phosphoric esters of polyethoxylated alcohols or phenols, fatty esters of polyols, and derivatives of the compounds containing sulphates, sulphonates and phosphates, for example alkylaryl polyglycol ethers, alkylsulphonates, alkyl sulphates, arylsulphonates, protein hydrolysates, lignosulphite waste liquors and methylcellulose. The presence of a surfactant is required if one of the active compounds and/or one of the inert carriers is insoluble in water and the application is carried out in water. The proportion of surfactants is between 5 and 40 percent by weight of the compositions according to the invention.

It is possible to use colorants such as inorganic pigments, for example iron oxide, titanium oxide, Prussian blue, and organic dyes, such as alizarin dyes, azo dyes and metal phthalocyanine dyes, and trace nutrients, such as salts of iron, manganese, boron, copper, cobalt, molybdenum and zinc.

Other possible additives are perfumes, mineral or vegetable oils, if appropriate modified, waxes and nutrients (including trace nutrients), such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present.

If appropriate, it is also possible for other additional components to be present, for example protective colloids, binders, glues, thickeners, thixotropic agents, penetrants, stabilizers, sequestrants, complex fomers. In general, the active compounds can be combined with any solid or liquid additive customarily used for formulation purposes.

The formulations generally comprise between 0.05 and 99% by weight, 0.01 and 98% by weight, preferably between 0.1 and 95% by weight, particularly preferably between 0.5 and 90% by weight, of active compound, very particularly preferably between 10 and 70 percent by weight.

The formulations described above can be employed in a method according to the invention for controlling unwanted microorganisms where the diaminopyrimidines according to the invention are applied to the microorganisms and/or their habitat.

The active compounds according to the invention, as such or in their formulations, can also be used in a mixture with known fungicides, bactericides, acaricides, nematicides or insecticides, for example to broaden the activity spectrum or to prevent the development of resistance.

Suitable mixing partners are, for example, known fungicides, insecticides, acaricides, nematicides or else bactericides (see also Pesticide Manual, 13th ed.).

A mixture with other known active compounds, such as herbicides, or with fertilizers and growth regulators, safeners and/or semiochemicals is also possible.

Application is carried out in a manner adapted to the use forms.

The control of phytopathogenic harmful fungi which damage plants post-emergence is primarily by treating the soil and the above-ground parts of the plants with crop protection compositions. Owing to concerns with a view to a possible impact of the crop protection compositions on the environment and human and animal health, there are efforts to reduce the amount of active compounds applied.

The active compounds can be applied as such, in the form of their formulations and the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, painting-on, etc. It is also possible to apply the active compounds by the ultra-low-volume method or to inject the preparation of active compound or the active compound itself into the soil. It is also possible to treat the seed of the plants.

When using the active compounds according to the invention as fungicides, the application rates can be varied within a relatively wide range, depending on the type of application. The application rate of the active compounds according to the invention is
in the treatment of parts of plants, for example leaves: from 0.1 to 10 000 g/ha, preferably from 10 to 1000 g/ha, particularly preferably from 50 to 300 g/ha (when the application is by watering or dripping, it is possible to reduce the application rate even more, in particular when inert substrates such as rock wool or perlite are used);
in the treatment of seed: from 2 to 200 g per 100 kg of seed, preferably from 3 to 150 g per 100 kg of seed, particularly preferably from 2.5 to 25 g per 100 kg of seed, very particularly preferably from 2.5 to 12.5 g per 100 kg of seed;
in soil treatment: from 0.1 to 10 000 g/ha, preferably from 1 to 5000 g/ha.

These application rates are mentioned only in an exemplary manner and not limiting for the purpose of the invention.

The compounds according to the invention can also be used for protecting objects which come into contact with salt water or brackish water, such as hulls, screens, nets, buildings, moorings and signalling systems, against colonization.

The active compounds according to the invention, alone or in combination with other active compounds, can furthermore be employed as antifouling agents.

The treatment method according to the invention can be used for treating genetically modified organisms (GMOs), for example plants or seeds. Genetically modified plants (or transgenic plants) are plants in which a heterologous gene has been stably integrated into the genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which is/are present in the plant (using for example, antisense technology, cosuppression technology or RNA interference RNAi technology). A heterologous gene that is located in the genome is also called a transgene. A transgene that is defined by its particular location in the plant genome is called a transformation or transgenic event.

Depending on the plant species or plant cultivars, their location and growth conditions (soils, climate, vegetation period, diet), the treatment according to the invention may also result in superadditive (“synergistic”) effects. Thus, for example, the following effects, which exceed the effects which were actually to be expected, are possible: reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf colour, earlier flowering, higher quality and/or a higher nutritional value of the harvested products, higher sugar concentration within the fruits, better storage stability and/or processability of the harvested products.

In the present case, unwanted phytopathogenic fungi and/or microorganisms and/or viruses are to be understood as meaning phytopathogenic fungi, bacteria and viruses. Thus, the substances according to the invention can be employed for protecting plants against attack by the abovementioned pathogens within a certain period of time after the treatment. The period of time within which protection is effected generally extends from 1 to 10 days, preferably 1 to 7 days, after the treatment of the plants with the active compounds.

Plants and plant cultivars which are preferably treated according to the invention include all plants with genetic material which bestows upon these plants particularly advantageous useful properties (whether this was achieved by breeding and/or biotechnology is immaterial).

Plants and plant cultivars which are also preferably treated according to the invention are resistant against one or more biotic stress factors, i.e. said plants have a better defence against animal and microbial pests, such as against nematodes, insects, mites, phytopathogenic fungi, bacteria, viruses and/or viroids.

Plants and plant cultivars which may also be treated according to the invention are those plants which are resistant to one or more abiotic stress factors. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients or shade avoidance.

Plants and plant cultivars which may also be treated according to the invention are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore by affected by improved plant architecture (under stress and non-stress conditions), including early flowering, flowering control for hybrid seed production, seedling vigour, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristic of heterosis or the hybrid effect which results in generally higher yield, vigour, health and resistance towards biotic and abiotic stress factors. Such plants are typically made by crossing an inbred male sterile parent line (the female parent) with another inbred male fertile parent line (the male parent). Hybrid seed is typically harvested from the male sterile plants and sold to growers. Male sterile plants can sometimes (e.g. in corn) be produced by detasseling, (i.e. the mechanical removal of the male reproductive organs or male flowers) but, more typically, male sterility is the result of genetic determinants in the plant genome. In that case, and especially when seed is the desired product to be harvested from the hybrid plants, it is typically useful to ensure that male fertility in the hybrid plants, which contain the genetic determinants responsible for male sterility, is fully restored. This can be accomplished by ensuring that the male parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described in Brassica species. However, genetic determinants for male sterility can also be located in the nuclear genome. Male sterile plants can also be obtained by plant biotechnology methods such as genetic engineering. A particularly useful means of obtaining male sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar.

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium, the CP4 gene of the bacterium Agrobacterium sp., the genes encoding a petunia EPSPS, a tomato EPSPS, or an Eleusine EPSPS. It can also be a mutated EPSPS. Glyphosate-tolerant plants can also be obtained by expressing a gene that encodes a glyphosate oxidoreductase enzyme. Glyphosate-tolerant plants can also be obtained by expressing a gene that encodes a glyphosate acetyl transferase enzyme. Glyphosate-tolerant plants can also be obtained by selecting plants containing naturally-occurring mutations of the above-mentioned genes.

Other herbicide-resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One such efficient detoxifying enzyme is, for example, an enzyme encoding a phosphinothricin acetyltransferase (such as the bar or pat protein from Streptomyces species). Plants expressing an exogenous phosphinothricin acetyltransferase have been described.

Further herbicide-tolerant plants are also plants that are made tolerant to the herbicides inhibiting the enzyme hydroxyphenylpyruvatedioxygenase (HPPD). Hydroxyphenylpyruvatedioxygenases are enzymes that catalyse the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated HPPD enzyme. Tolerance to HPPD-inhibitors can also be obtained by transforming plants with genes encoding certain enzymes enabling the formation of homogentisate despite the inhibition of the native HPPD enzyme by the HPPD-inhibitor. Tolerance of plants to HPPD inhibitors can also be improved by transforming plants with a gene encoding an enzyme prephenate dehydrogenase in addition to a gene encoding an HPPD-tolerant enzyme.

Still further herbicide-resistant plants are plants that are made tolerant to acetolactate synthase (ALS) inhibitors. Known ALS-inhibitors include, for example, sulphonylurea, imidazolinone, triazolopyrimidines, pyrimidinyloxy(thio)benzoates, and/or sulphonylaminocarbonyltriazolinone herbicides. Different mutations in the ALS enzyme (also known as acetohydroxyacid synthase, AHAS) are known to confer tolerance to different herbicides and groups of herbicides. The production of sulphonylurea-tolerant plants and imidazolinone-tolerant plants has been described in the international publication WO 1996/033270. Further sulphonylurea- and imidazolinone-tolerant plants have also been described, for example in WO 2007/024782.

Other plants tolerant to imidazolinone and/or sulphonylurea can be obtained by induced mutagenesis, by selection in cell cultures in the presence of the herbicide or by mutation breeding.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

In the present context, the term “insect-resistant transgenic plant” includes any plant containing at least one transgene comprising a coding sequence encoding:
1) an insecticidal crystal protein from Bacillus thuringiensis or an insecticidal portion thereof, such as the insecticidal crystal proteins listed online at: http://www.lifesci.sussex.ac.uk/Home/Neil Crickmore/Bt/, or insecticidal portions thereof, for example proteins of the Cry protein classes Cry1Ab, Cry1Ac, Cry1F, Cry2Ab, Cry3Ae or Cry3Bb or insecticidal portions thereof; or
2) a crystal protein from Bacillus thuringiensis or a portion thereof which is insecticidal in the presence of a second other crystal protein from Bacillus thuringiensis or a portion thereof, such as the binary toxin made up of the Cy34 and Cy35 crystal proteins; or
3) a hybrid insecticidal protein comprising parts of two different insecticidal crystal proteins from Bacillus thuringiensis, such as a hybrid of the proteins of 1) above or a hybrid of the proteins of 2) above, for example the Cry1A.105 protein produced by maize event MON98034 (WO 2007/027777); or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation, such as the Cry3Bb1 protein in maize events MON863 or MON88017, or the Cry3A protein in maize event MIR604;
5) an insecticidal secreted protein from Bacillus thuringiensis or Bacillus cereus, or an insecticidal portion thereof, such as the vegetative insecticidal proteins (VIP) listed at: http://www.lifesci.sussex.ac.uk/home/Neil_Crickmore/Bt/vip.html, for example proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1a and VIP2A proteins;
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation (while still encoding an insecticidal protein), such as the VIP3Aa protein in cotton event COT102.

Of course, insect-resistant transgenic plants, as used herein, also include any plant comprising a combination of genes encoding the proteins of any one of the above classes 1 to 8. In one embodiment, an insect-resistant plant contains more than one transgene encoding a protein of any one of the above classes 1 to 8, to expand the range of target insect species affected or to delay insect resistance development to the plants, by using different proteins insecticidal to the same target insect species but having a different mode of action, such as binding to different receptor binding sites in the insect.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance. Particularly useful stress tolerance plants include:
a. plants which contain a transgene capable of reducing the expression and/or the activity of the poly(ADP-ribose)polymerase (PARP) gene in the plant cells or plants.
b. plants which contain a stress tolerance-enhancing transgene capable of reducing the expression and/or the activity of the PARG encoding genes of the plants or plant cells;
c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyl transferase, nicotinamide adenine dinucleotide synthetase or nicotinamide phosphoribosyltransferase.

Plants or plant cultivars (obtained by plant biotechnology methods such as genetic engineering) which may also be treated according to the invention show altered quantity, quality and/or storage-stability of the harvested product and/or altered properties of specific ingredients of the harvested product such as, for example:
1) transgenic plants which synthesize a modified starch, which in its physical-chemical characteristics, in particular the amylose content or the amylose/amylopectin ratio, the degree of branching, the average chain length, the side chain distribution, the viscosity behaviour, the gelling strength, the starch grain size and/or the starch grain morphology, is changed in comparison with the synthesized starch in wild type plant cells or plants, so that this modified starch is better suited for special applications.
2) transgenic plants which synthesize non-starch carbohydrate polymers or which synthesize non-starch carbohydrate polymers with altered properties in comparison to wild type plants without genetic modification. Examples are plants which produce polyfructose, especially of the inulin and levan type, plants which produce alpha-1,4-glucans, plants which produce alpha-1,6 branched alpha-1,4-glucans, and plants producing alternan.
3) transgenic plants which produce hyaluronan.

Plants or plant cultivars (that can be obtained by plant biotechnology methods such as genetic engineering) which may also be treated according to the invention are plants, such as cotton plants, with altered fibre characteristics. Such plants can be obtained by genetic transformation, or by selection of plants containing a mutation imparting such altered fibre characteristics and include:
a) plants, such as cotton plants, which contain an altered form of cellulose synthase genes,
b) plants, such as cotton plants, which contain an altered form of rsw2 or rsw3 homologous nucleic acids;
c) plants, such as cotton plants, with an increased expression of sucrose phosphate synthase;
d) plants, such as cotton plants, with an increased expression of sucrose synthase;
e) plants, such as cotton plants, wherein the timing of the plasmodesmatal gating at the basis of the fibre cell is altered, for example through downregulation of fibre-selective β-1,3-glucanase;
f) plants, such as cotton plants, which have fibres with altered reactivity, for example through the expression of the N-acetylglucosaminetransferase gene including nodC and chitin synthase genes.

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation or by selection of plants containing a mutation imparting such altered oil characteristics and include:
a) plants, such as oilseed rape plants, which produce oil having a high oleic acid content;
b) plants, such as oilseed rape plants, which produce oil having a low linolenic acid content;
c) plants, such as oilseed rape plants, which produce oil having a low level of saturated fatty acids.

Particularly useful transgenic plants which may be treated according to the invention are plants which comprise one or more genes which encode one or more toxins, are the following which are sold under the trade names YIELD GARD® (for example maize, cotton, soya beans), KnockOut® (for example maize), BiteGard® (for example maize), Bt-Xtra® (for example maize), StarLink® (for example maize), Bollgard® (cotton), Nucotn® (cotton), Nucotn 33B® (cotton), NatureGard® (for example maize), Protecta® and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya beans), Liberty Link® (tolerance to phosphinothricin, for example oilseed rape), IMI® (tolerance to imidazolinone) and SCS® (tolerance to sulphonylurea, for example maize). Herbicide-resistant plants (plants bred in a conventional manner for herbicide tolerance) which may be mentioned include the varieties sold under the name Clearfield® (for example maize).

Particularly useful transgenic plants which may be treated according to the invention are plants containing transformation events, or a combination of transformation events, that are listed for example in the databases for various national or regional regulatory agencies (see for example http://gmoinfo.jrc.it/gmp_browse.aspx and http://www.agbios.com/dbase.php).

According to the invention, the plants listed can be treated particularly advantageously with the compounds of the general formula (I) or the active compound mixtures according to the invention. The preferred ranges indicated above for the active compounds and mixtures also apply to the treatment of these plants. Particular emphasis is given to treating the plants with the compounds and mixtures specifically indicated in the present text.

The compositions or active compounds according to the invention can also be used to protect plants for a certain period after treatment against attack by the pathogens mentioned. The period for which protection is provided generally extends over 1 to 28 days, preferably over 1 to 14 days, particularly preferably over 1 to 10 days, very particularly preferably over 1 to 7 days, after the treatment of the plants with the active compounds, or over up to 200 days after seed treatment.

Preparation and use of the active compounds of the formulae (I) according to the invention is shown in the examples below. However, the invention is not limited to these examples.

### Preparation of Starting Materials of the Formula (V)

### 2,5-Dichloro-N-cyclobutylpyrimidin-4-amine

At −10° C., 3.39 g (24.5 mmol) of potassium carbonate are added to a solution of 3.00 g (16.4 mmol) of 2,4,5-trichloropyrimidine in 50 ml of acetonitrile. 1.22 g (17.2 mmol) of cyclobutylamine are then added dropwise as a 20% strength solution in acetonitrile. With stirring, the reaction mixture is allowed to warm to room temperature overnight. The reaction mixture is stirred into 250 ml of ice-water/dilute hydrochloric acid (1:1). The mixture is extracted with ethyl acetate (2×200 ml), the combined organic phases are then washed with water (2×100 ml) and dried over MgSO₄ and the solvent is removed under reduced pressure. This gives 3.45 g (94%) of 2,5-dichloro-N-cyclobutylpyrimidin-4-amine (V-1) (logP (pH 2.3): 2.62).

¹H NMR (400 MHz, MeCN-d) δ=8.00 (s, 1H), 6.31 (br. s, 1H), 4.54-4.46 (m, 1H), 2.39-2.31 (m, 2H), 2.15-2.04 (m, 2H), 1.83-1.77 (m, 2H).

The compounds below can be prepared in an analogous manner:

### 5-Bromo-2-chloro-N-cyclobutylpyrimidin-4-amine logP (pH 2.3): 2.87

¹H NMR (400 MHz, DMSO-d) δ=8.20 (s, 1H), 7.52 (br. s, 1H), 4.45 (br. m, 1H), 2.24 (m, 2H), 2.17 (m, 2H), 1.69 (m, 2H).

### 2,5-Dichloro-N-cyclopropylpyrimidin-4-amine logP (pH 2.3): 1.79.

¹H NMR (400 MHz, DMSO-d) δ=8.11 (s, 1H), 7.71 (br. s, 1H), 2.89-2.84 (m, 1H), 0.79-0.64 (m, 4H).

### 5-Bromo-2-chloro-N-cyclopropylpyrimidin-4-amine logP (pH 2.3): 1.97

¹H NMR (400 MHz, MeCN-d) δ=8.12 (s, 1H), 6.17 (br. s, 1H), 2.87-2.80 (m, 1H), 0.85-0.79 (m, 2H) 0.66-0.62 (m, 2H).

### 2-Chloro-N-cyclopropyl-5-fluoropyrimidin-4-amine logP (pH 2.3): 1.42

(CAS: 893772-23-1 commercially available from Aurora Screening Library, Graz, Austria)

### 2-Chloro-N-cyclopropyl-5-methylpyrimidin-4-amine logP (pH 2.3): 1.28

### 2-Chloro-N-cyclopropyl-5-iodopyrimidin-4-amine logP (pH 2.3): 2.19

¹H NMR (400 MHz, MeCN-d) δ=8.28 (s, 1H), 5.96 (br. s, 1H), 2.85-2.80 (m, 1H), 0.84-0.79 (m, 2H) 0.64-0.61 (m, 2H).

### 2,5-Dichloro-N-(cyclopropylmethyl)pyrimidin-4-amine logP (pH 2.3): 2.51

¹H NMR (400 MHz, MeCN-d) δ=8.01 (s, 1H), 6.34 (br. s, 1H), 3.33-3.29 (m, 2H), 1.16-1.06 (m, 1H), 0.54-0.45 (m, 2H) 0.33-0.24 (m, 2H).

### 2,5-Dichloro-N-(2,2-difluoroethyl)pyrimidin-4-amine logP (pH 2.3): 1.96

¹H NMR (400 MHz, MeCN-d) δ=8.10 (s, 1H), 6.47 (br. s, 1H), 6.02 (tt, 1H), 3.86 (m, 2H).

### 2,5-Dichloro-N-(2,2,2-trifluoroethyl)pyrimidin-4-amine logP (pH 2.3): 2.26

¹H NMR (400 MHz, DMSO-d) δ=8.29 (s, 1H), 8.25 (br. s, 1H), 4.24-4.15 (m, 2H).

### 2,5-Dichloro-N-(3-methoxypropan-2-yl)pyrimidin-4-amine logP (pH 2.3): 2.10

¹H NMR (400 MHz, MeCN-d) δ=8.02 (s, 1H), 6.03 (br. s, 1H), 4.39-4.33 (m, 1H), 3.48-3.40 (m, 2H), 3.33 (s., 3H), 1.23 (d, 3H).

### 2,5-Dichloro-N-(prop-2-yn-1-yl)pyrimidin-4-amine logP (pH 2.3): 2.12

¹H NMR (400 MHz, MeCN-d) δ=8.03 (s, 1H), 6.40 (br. s, 1H), 5.98-5.88 (m, 1H), 5.23-5.12 (m, 2H), 4.09-4.06 (m, 2H).

### 2,5-Dichloro-N-cyclopropyl-N-methylpyrimidin-4-amine logP (pH 2.3): 2.82

¹H NMR (400 MHz, MeCN-d) δ=8.09 (s, 1H), 3.15-3.12 (m, 1H), 3.11 (s, 3H), 0.87-0.82 (m, 2H), 0.72-0.70 (m, 2H).

### 2,5-Dichloro-N-(2-methylcyclopropyl)pyrimidin-4-amine logP (pH 2.3): 2.53

¹H NMR (400 MHz, DMSO-d, Main isomer) δ=8.10 (s, 1H), 7.49 (s, 1H), 2.48-2.49 (m, 1H), 1.09 (d, 3H), 0.96-1.02 (m, 1H), 0.81-0.85 (m, 1H), 0.53-0.58 (m, 1H).

### 5-Bromo-2-chloro-N-(2-methylcyclopropyl)pyrimidin-4-amine logP (pH 2.3): 2.68

¹H NMR (400 MHz, DMSO-d, Main isomer) δ=8.19 (s, 1H), 7.71 (s, 1H), 1.09 (d, 3H), 0.90-1.06 (m, 2H), 0.81-0.86 (m, 1H), 0.53-0.58 (m, 1H).

### 2-Chloro-N-(2-methylcyclopropyl)-5-(trifluoromethyl)pyrimidin-4-amine logP (pH 2.3): 3.02

¹H NMR (600 MHz, DMSO-d, Main isomer) δ=8.39 (s, 1H), 8.00 (s, 1H), 1.10 (d, 3H), 0.84-1.08 (m, 3H), 0.57-0.66 (m, 1H).

2,5-Dichloro-N-(2-ethylcyclopropyl)pyrimidin-4-amine logP (pH 2.3): 3.10; ¹H NMR (400 MHz, DMSO-d₆, Main isomer) δ=8.10 (s, 1H), 7.70 (s, 1H), 2.48-2.56 (m, 1H), 1.25-1.40 (m, 2H), 1.00-1.04 (q, 2H), 0.85-0.77 (m, 1H), 0.82-0.84 (m, 1H), 0.56-0.60 (m, 1H).

### 2,5-Dichloro-N-(propan-2-yl)pyrimidin-4-amine logP (pH 2.3): 2.46

¹H NMR (400 MHz, MeCN-d) δ=7.99 (s, 1H), 5.92 (br. s, 1H), 4.31-4.23 (m, 1H), 1.25 (d, 6 H)

### 2-Chloro-N-cyclobutyl-5-trifluoromethylpyrimidin-4-amine

A mixture of 8.07 g (37.2 mmol) of 2,4-dichloro-5-trifluoromethylpyrimidine and 12.8 g (92.9 mmol) of potassium carbonate in 150 ml of acetonitrile is warmed to 50° C. 4.00 g (37.2 mmol) of cyclobutylamine hydrochloride are then added, and the mixture is stirred for 2 h. After cooling, the reaction mixture is stirred into 500 ml of ice-water and extracted with ethyl acetate (3×200 ml). The combined organic phases are separated off, washed with water (2×250 ml), dried over MgSO₄ and freed from the solvent under reduced pressure. The crude product is purified by column chromatography on silica gel (cyclohexane/ethyl acetate). This gives 4.00 g (41%) of 2-chloro-N-cyclobutyl-5-trifluoromethylpyrimidin-4-amine (V-4). logP (pH 2.3): 3.20

¹H NMR (400 MHz, MeCN-d) δ=8.27 (s, 1H), 6.19 (br. s, 1H), 4.64-4.56 (m, 1H), 2.40-2.32 (m, 2H), 2.14-2.04 (m, 2H), 1.82-1.74 (m, 2H).

The following compounds can be prepared in an analogous manner:

### 2-Chloro-N-cyclopropyl-5-trifluoromethylpyrimidin-4-amine logP (pH 2.3): 2.39

¹H NMR (400 MHz, MeCN-d) δ=8.28 (s, 1H), 6.34 (br. s, 1H), 2.91-2.86 (m, 1H), 0.85-0.80 (m, 2H), 0.66-0.62 (m, 2H).

### 2-Chloro-N-(cyclopropylmethyl)-5-trifluoromethylpyrimidin-4-amine logP (pH 2.3): 3.40

¹H NMR (400 MHz, DMSO-d6) δ=8.05 (s, 1H), 7.51 (br. s., 1H), 3.02 (t, 2H), 0.79-0.89 (m, 1 H), 0.11-0.17 (m, 2H), −0.03-0.03 (m, 2H); M+H=252.0.

### Preparation of Compounds of the Formula (Vi)

### Method A:

### 3-{[5-Chloro-4-(cyclopropylamino)pyrimidin-2-yl]amino}benzenecarbaldehyde

A mixture of 15 g (73.51 mmol) of 2,5-dichloro-N-cyclopropylpyrimidin-4-amine, 15.98 g (95.56 mmol) of 3-(dimethoxymethyl)aniline and 12.243 g (58.81 mmol) of 4-toluenesulphonic acid in 350 ml of dioxane is stirred at 105° C. for 16 h. After cooling, the reaction mixture is freed from the solvent under reduced pressure and the residue is neutralized with saturated NaHCO₃ solution and extracted with ethyl acetate. The organic phase is dried over MgSO₄ and the solvent is removed under reduced pressure. This gives 20.31 g of the desired product (logP (pH 2.7): 1.82).

¹H NMR (400 MHz, DMSO-d) δ=9.94 (s, 1H), 9.36 (s, 1H), 8.54 (s, 1H), 8.05-7.98 (m, 1H), 7.95 (s, 1H), 7.50-7.41 (m, 2H), 7.08 (s, 1H), 2.95-2.86 (m, 1H), 0.86-0.78 (m, 2H), 0.70-0.63 (m, 2H)

The following compounds can be prepared in an analogous manner:

### 3-{[5-Bromo-4-(cyclopropylamino)pyrimidin-2-yl]amino}benzenecarbaldehyde logP (pH 2.7): 1.93

¹H NMR (400 MHz, DMSO-d) δ=9.94 (s, 1H), 9.38 (s, 1H), 8.54 (s, 1H), 8.06-7.99 (m, 2H), 7.50-7.43 (m, 2H), 6.84 (s, 1H), 2.94-2.84 (m, 1H), 0.86-0.78 (m, 2H), 0.70-0.62 (m, 2H)

### 3-{[5-Chloro-4-(cyclobutylamino)pyrimidin-2-yl]amino}benzenecarbaldehyde logP (pH 2.7): 2.16

¹H NMR (400 MHz, DMSO-d) δ=9.98 (s, 1H), 9.30 (s, 1H), 8.48 (s, 1H), 7.95 (s, 1H), 7.90-7.85 (m, 1H), 7.50-7.45 (m, 2H), 7.05 (d, 1H), 4.69-4.55 (m, 1H), 2.38-2.26 (m, 2H), 2.20-2.07 (m, 2H), 1.70-1.68 (m, 2H)

### 3-{[5-Bromo-4-(cyclobutylamino)pyrimidin-2-yl]amino}benzenecarbaldehyde logP (pH 2.7): 2.35

¹H NMR (400 MHz, DMSO-d) (selected signals) δ=9.98 (s, 1H), 9.32 (s, 1H), 8.48 (s, 1H); MM+1: 348.1

### 3-({5-Chloro-4-[(cyclopropylmethyl)amino]pyrimidin-2-yl}amino)benzenecarbaldehyde logP (pH 2.7): 1.94

¹H NMR (400 MHz, DMSO-d) (selected signals) δ=9.95 (s, 1H), 9.28 (s, 1H), 8.42 (s, 1H); MM+1: 303.1

### 3-{[4-(Cyclopropylamino)-5-(trifluoromethyl)pyrimidin-2-yl]amino}benzenecarbaldehyde logP (pH 2.7): 2.73

¹H NMR (400 MHz, DMSO-d6) δ=9.96 (s, 1H), 9.76 (s, 1H), 8.57 (s, 1H), 8.20 (s, 1H), 8.10-8.02 (m, 1H), 7.55-7.49 (m, 2H), 6.93 (s, 1H), 2.99-2.90 (m, 1H), 0.88-0.70 (m, 2H), 0.70-0.62 (m, 2H)

### 3-{[4-(Cyclopropylamino)-5-iodopyrimidin-2-yl]amino}benzenecarbaldehyde logP (pH 2.7): 1.96

¹H NMR (400 MHz, DMSO-d) (selected signals) δ=9.94 (s, 1H), 9.36 (s, 1H), 8.53 (s, 1H), 8.15 (s, 1H); MM+1: 381.0

### 3-{[4-(Cyclobutylamino)-5-(trifluoromethyl)pyrimidin-2-yl]amino}benzenecarbaldehyde logP (pH 2.7): 3.26

¹H NMR (400 MHz, DMSO-d) δ=10.00 (s, 1H), 9.69 (s, 1H), 8.50 (s, 1H), 8.21 (s, 1H), 7.93-7.86 (m, 1H), 7.60-7.49 (m, 2H), 6.78 (d, 1H), 4.79-4.63 (m, 1H), 2.38-2.25 (m, 2H), 2.21-2.08 (m, 2H), 1.69-1.57 (m, 2H)

### 3-({5-Chloro-4-[cyclopropyl(methyl)amino]pyrimidin-2-yl}amino)benzenecarbaldehyde logP (pH 2.7): 2.84

¹H NMR (400 MHz, DMSO-d) (selected signals) δ=9.94 (s, 1H), 9.42 (s, 1H), 8.43 (s, 1H); MM+1: 303.1

### 3-({5-Chloro-4-[(2,2,2-trifluoroethyl)amino]pyrimidin-2-yl}amino)benzenecarbaldehyde logP (pH 2.7): 2.46

¹H NMR (400 MHz, acetonitrile-d) δ=9.97 (s, 1H), 8.32 (t, 1H), 8.05 (s, 1H), 8.04-7.97 (m, 1H), 7.84-7.76 (m, 1H), 7.55-7.45 (m, 2H), 6.40 (s, 1H), 4.32-4.20 (2H, m).

### 3-{[4-(Cyclopropylamino)-5-fluoropyrimidin-2-yl]amino}benzenecarbaldehyde logP (pH 2.7): 1.26

¹H NMR (400 MHz, acetonitrile-d) (selected signals) δ=9.96 (s, 1H), 8.44 (s, 1H), 7.94-7.87 (m, 1H); MM+1: 273.1

### 3-{[4-(Cyclopropylamino)-5-methylpyrimidin-2-yl]amino}benzenecarbaldehyde logP (pH 2.7): 1.13

¹H NMR (400 MHz, acetonitrile-d) (selected signals) δ=9.94 (s, 1H), 8.58 (s, 1H), 7.99-7.92 (m, 1H); MM+1: 269.1

### 3-{[5-Chloro-4-(prop-2-yn-1-ylamino)pyrimidin-2-yl]amino}benzenecarbaldehyde logP (pH 2.7): 1.74

¹H NMR (400 MHz, acetonitrile-d) (selected signals) δ=10.00 (s, 1H), 8.40 (s, 1H), 7.97 (s, 1H); MM+1: 287.1

### 3-({5-Chloro-4-[(1-methoxypropan-2-yl)amino]pyrimidin-2-yl}amino)benzenecarbaldehyde logP (pH 2.7): 1.80

¹H NMR (400 MHz, acetonitrile-d) (selected signals) δ=9.96 (s, 1H), 8.38 (s, 1H), 7.93 (s, 1H); MM+1: 321.1

### 3-({5-Chloro-4-[(2,2-difluoroethyl)amino]pyrimidin-2-yl}amino)benzenecarbaldehyde logP (pH 2.7): 2.01

¹H NMR (400 MHz, acetonitrile-d) δ=9.97 (s, 1H), 8.32 (s, 1H), 8.01 (s, 1H), 7.97 (s, 1H), 7.84-7.78 (m, 1H), 7.55-7.45 (m, 2H), 6.28 (s, 1H), 6.11 (tt, 1H), 3.95-3.80 (m, 2H)

### 3-((5-Chloro-4-[(2-methylcyclopropyl)amino]pyrimidin-2-yl)amino)benzaldehyde (logP (pH 2.3): 2.11

¹H NMR (400 MHz, DMSO-d, main isomer) δ=10.05 (s, 1H), 9.31 (s, 1H), 8.44 (s, 1H), 8.04-8.07 (m, 1H), 7.93 (s, 1H), 7.43-7.48 (m, 2H), 7.00 (s, 1H), 2.63-2.66 (m, 1H), 1.08 (d, 3H), 0.93-1.09 (m, 1H), 0.74-0.97 (m, 1H) 0.58-0.62 (m, 1H).

### 3-{[5-Chloro-4-(isopropylamino)pyrimidin-2-yl]amino}benzaldehyde logP (pH 2.7): 1.86

¹H NMR (400 MHz, DMSO-d6) (selected signals) δ=9.95 (s, 1H), 9.28 (s, 1H), 8.42 (s, 1H), 7.94 (s, 1H); MM+1: 291.1

### Method B

### 1-(3-{[5-Chloro-4-(cyclopropylamino)pyrimidin-2-yl]amino}phenyl)ethanone

A mixture of 2.3 g (11.3 mmol) of 2,5-dichloro-N-cyclopropylpyrimidin-4-amine, 1.9 g (14.1 mmol) of 1-(3-aminophenyl)ethanone and 388 mg (2.25 mmol) of 4-toluenesulphonic acid in 40 ml of dioxan is stirred at 105° C. for 16 h. After cooling, the reaction mixture is freed from the solvent under reduced pressure and the residue is neutralized with saturated NaHCO₃ solution and extracted with ethyl acetate. The organic phase is dried over MgSO₄ and the solvent is removed under reduced pressure. This gives 2.2 g of the desired product (logP (pH 2.7): 1.75).

¹H NMR (400 MHz, DMSO-d) δ=9.24 (s, br., 1H), 8.44 (s, br., 1H), 8.06-8.03 (m, 1H), 7.93 (s, 1H), 7.51-7.48 (m, 1H), 7.37 (t, 1H), 7.04 (s, br., 1H), 2.96-2.90 (m, 1H), 2.52 (s), 0.83-0.74 (m, 2H), 0.67-0.64 (m, 2H)

The following compounds can be prepared in an analogous manner:

### 1-(4-{[5-Chloro-4-(cyclopropylamino)pyrimidin-2-yl]amino}phenyl)ethanone logP (pH 2.7): 1.93

¹H NMR (400 MHz, DMSO-d) δ=9.51 (s, br., 1H), 7.99-7.97 (d, 3H), 7.87-7.84 (d, 2H), 7.14 (s, br., 1H), 3.91-2.85 (m, 1H), 2.48 (s), 0.85-0.80 (m, 2H), 0.70-0.68 (m, 2H)

### 2-Chloro-{[5-chloro-4-(cyclopropylamino)pyrimidin-2-yl]amino}benzenecarbaldehyde logP (pH 2.7): 2.68

¹H NMR (400 MHz, DMSO-d) δ=10.31 (s, 1H), 9.47 (s, 1H), 8.54 (d, 1H), 8.02 (dd, 1H), 7.95 (s, 1H), 7.46 (d, 1H), 7.11 (s, 1H), 2.95-2.86 (m, 1H), 0.86-0.80 (m, 2H), 0.68-0.61 (m, 2H)

### 1-(3-{[4-(Cyclopropylamino)-5-(trifluoromethyl)pyrimidin-2-yl]amino}phenyl)ethanone logP (pH 2.7): 2.72

¹H NMR (400 MHz, DMSO-d) δ=9.64 (s, br., 1H), 8.47 (s, br., 1H), 8.20 (s, 1H), 8.11-8.09 (m, 1H), 7.58-7.55 (m, 1H), 7.42 (t, 1H), 6.87 (s, br., 1H), 3.00-2.93 (m, 1H), 2.53 (s, 3H), 0.84-0.76 (m, 2H), 0.72-0.64 (m, 2H)

### 1-(3-{[4-(Cyclobutylamino)-5-(trifluoromethyl)pyrimidin-2-yl]amino}phenyl)ethanone logP (pH 2.7): 3.24

¹H NMR (400 MHz, DMSO-d) δ=9.58 (s, br., 1H), 8.47 (s, br., 1H), 8.20 (s, 1H), 7.89-7.86 (m, 1H), 7.60-7.57 (d, 1H), 7.42 (t, 1H), 6.75-6.74 (d, br., 1H), 4.75-4.69 (m, 1H), 2.57 (s, 3H), 2.35-2.25 (m, 2H), 2.19-2.09 (m, 2H), 1.75-1.67 (m, 2H)

### 1-(3-{[5-Chloro-4-(cyclopropylamino)pyrimidin-2-yl]amino}phenyl)propan-1-one logP (pH 2.7) 2.25

¹H NMR (400 MHz, DMSO-d) δ=9.22 (s, br., 1H), 8.42 (s, br., 1H), 8.07-8.04 (m, 1H), 7.93 (s, 1H), 7.50-7.48 (m, 1H), 7.37 (t, 1H), 7.02 (s, br., 1H), 2.99-2.90 (m, 3H), 1.09 (s, 3H), 0.82-0.78 (m, 2H), 0.68-0.64 (m, 2H)

### 1-[3-({4-[(Cyclopropylmethyl)amino]-5-(trifluoromethyl)pyrimidin-2-yl}amino)phenyl]-ethanone logP (pH 2.7): 2.77

¹H NMR (400 MHz, DMSO-d) δ=9.56 (s, br., 1H), 8.41 (s, br., 1H), 8.20 (s, br., 1H), 7.94-7.91 (m, 1H), 7.58-7.55 (m, 1H), 7.41 (t, 1H), 6.88 (s, br., 1H), 3.42-3.37 (m, 2H), 2.56 (s, 3H), 1.21-1.17 (m, 1H), 0.43-0.38 (m, 2H), 0.28-0.25 (m, 2H)

### Preparation of Compounds of the Formula (Ia)

### Method C:

### 5-Chloro-N⁴-cyclopropyl-N²-[3-(1,3-dioxolan-2-yl)phenyl]pyrimidine-2,4-diamine (Example 1)

At 20° C., a mixture of 10 g (34.63 mmol) of 3-{[5-chloro-4-(cyclopropylamino)pyrimidin-2-yl]amino}benzenecarbaldehyde and 43 g of 1,2-ethanediol (692.7 mmol) is initially charged in 200 ml of 1,4-dioxane, 17.32 ml of 4N HCl (69.267 mmol) in 1,4-dioxane are added and stirring at 20° C. is continued overnight. The reaction mixture is then freed from the solvent under reduced pressure and the residue is stirred with a mixture of sodium bicarbonate solution and ethyl acetate. The organic phase is dried over MgSO₄ and freed from the solvent under reduced pressure. The crude product is purified by column chromatography on alumina using the mobile phase cyclohexane/ethyl acetate. This gives 5.8 g of the desired product (logP (pH 2.7): 1.59).

¹H NMR (400 MHz, DMSO-d) δ=9.07 (s, 1H), 8.02 (t, 1H), 7.91 (s, 1H), 7.77-7.21 (m, 1H), 7.23 (t, 1H), 7.03-6.93 (m, 2H), 5.67 (s, 1H), 4.03-3.89 (m, 4H), 2.94-2.85 (m, 1H), 0.85-0.77 (m, 2H), 0.69-0.60 (m, 1H)

### 5-Chloro-N⁴-cyclopropyl-N²-[3-(2-methyl-1,3-dioxolan-2-yl)phenyl]pyrimidine-2,4-diamine (Example 12)

At 110° C., 0.50 g (1.65 mmol) of 1-(3-{[5-chloro-4-(cyclopropylamino)pyrimidin-2-yl]amino}phenyl)ethanone, 0.51 g of 1,2-ethanediol (8.3 mmol) and 100 mg of 4-toluenesulphonic acid in 40 ml of toluene are heated on a water separator for 24 hours. After cooling, the reaction mixture is freed from the solvent under reduced pressure and the residue is stirred with a mixture of ice-cold sodium bicarbonate solution and ethyl acetate. The organic phase is dried over MgSO₄ and freed from the solvent under reduced pressure. The crude product is triturated with a mixture of tert-butyl methyl ether and petroleum ether 1:2. This gives 300 mg of the desired product (logP (pH 2.7): 1.90);

¹H NMR (400 MHz, DMSO-d) δ=9.02 (s, br., 1H), 8.04-8.03 (m, 1H), 7.9 (s, 1H), 7.71-7.69 (m, 1H), 7.19 (t, 1H), 6.97-6.94 (m, 2H), 3.96-3.92 (m, 2H), 3.71-3.68 (m, 2H), 2.98-2.91 (m, 1H), 1.53 (s, 3H), 0.83-0.76 (m, 2H), 0.67-0.65 (m, 2H)

### 5-Chloro-N⁴-cyclopropyl-N²-[4-(2-methyl-1,3-dioxolan-2-yl)phenyl]pyrimidine-2,4-diamine (Example 27)

At 110° C., 0.50 g (1.65 mmol) of 1-(4-{[5-chloro-4-(cyclopropylamino)pyrimidin-2-yl]amino}-phenyl)ethanone, 0.51 g of 1,2-ethanediol (8.3 mmol) and 100 mg of 4-toluenesulphonic acid in 40 ml of toluene are heated on a water separator for 24 hours. After cooling, the reaction mixture is freed from the solvent under reduced pressure and the residue is stirred with a mixture of ice-cold sodium bicarbonate solution and ethyl acetate. The organic phase is dried over MgSO₄ and freed from the solvent under reduced pressure. The crude product is triturated with a mixture of tert-butyl methyl ether and petroleum ether 1:2. This gives 500 mg of the desired product (logP (pH 2.7): 1.84);

¹H NMR (400 MHz, DMSO-d) δ=9.03 (s, br., 1H), 7.90 (s, 1H), 7.81-7.79 (d, 2H), 7.29-7.26 (m, 2H), 6.98 (s, br., 1H), 3.99-3.93 (m, 2H), 3.73-3.70 (m, 2H), 2.89-2.83 (m, 1H), 1.54 (s, 3H), 0.83-0.76 (m, 2H), 0.68-0.66 (m, 2H)

### Method D:

### 5-Chloro-N⁴-cyclopropyl-N²-(3-{4-[(diethylamino)methyl]-1,3-dioxolan-2-yl}phenyl)-pyrimidine-2,4-diamine (Example 40)

A mixture of 150 mg (0.52 mmol) of 3-{[5-chloro-4-(cyclopropylamino)pyrimidin-2-yl]amino}benzenecarbaldehyde and 0.765 g of 3-diethylamino-1,2-propanediol (5.195 mmol) and 811 mg (5.715 mmol) of BF₃.Et₂O is stirred at 20° C. for 16 hours. The mixture is then neutralized with saturated NaHCO₃ solution and extracted with dichloromethane, and the organic phase is washed once with 10 ml of water. The organic phase is dried over MgSO₄ and freed from the solvent under reduced pressure. The crude product is purified by column chromatography on silica gel (RS70 ND100-20) using water/acetonitrile. This gives 88 mg of the desired product as a mixture of two stereoisomers (logP (pH 2.7): 1.05).

¹H NMR (400 MHz, DMSO-d) (2 diastereoisomers) δ=9.08 (s, 1H), [8.01, 7.96] (s, 1H), 7.90 (s, 1H), 7.80-7.70 (m, 1H), 7.36-7.28 (m, 1H), 7.03-6.90 (m, 2H), [5.78, 5.67] (s, 1H), 4.20-3.98 (m, 2H), 3.72-3.60 (m, 1H), 2.95-2.85 (m, 1H), 2.70-2.45 (m), 0.95 (q, 6H), 0.83-0.75 (m, 2H), 0.68-0.62 (m, 2H)

### Method E:

### 5-Chloro-N⁴-cyclopropyl-N²-[3-(1,3-dioxan-2-yl)phenyl]pyrimidine-2,4-diamine (Example 5)

A mixture of 150 mg (0.735 mmol) of 2,5-dichloro-N-cyclopropylpyrimidin-4-amine, 158.1 mg (0.882 mmol) of 3-(1,3-dioxan-2-yl)aniline and 107.6 mg (0.625 mmol) of 4-toluenesulphonic acid in 6 ml of dioxane is stirred at 105° C. for 24 h. The mixture is then concentrated using a rotary evaporator, the residue is neutralized with saturated NaHCO₃ solution and extracted with 20 ml of ethyl acetate and the organic phase is dried over MgSO₄ and concentrated on a rotary evaporator. The crude product is purified by column chromatography on alumina (cyclohexane/ethyl acetate). This gives 178.5 mg of the desired product (logP (pH 2.7): 1.69).

¹H NMR (400 MHz, DMSO-d) δ=9.04 (s, 1H), 8.11 (s, 1H), 7.90 (s, 1H), 7.62 (dd 1H), 7.18 (t, 1H), 6.92-6.98 (m, 2H), 4.07-4.11 (m, 6H), 3.87-3.94 (m, 2H), 1.90-1.98 (m, 1H), 1.55-1.61 (m, 1H), 1.40-1.44 (m, 1H), 0.79-0.84 (m, 1H), 0.63-0.67 (m, 1H).

### Preparation of Compounds of the Formula (Ib)

### 5-Chloro-N⁴-cyclopropyl-N²-[3-(1,3-dithian-2-yl)phenyl]pyrimidine-2,4-diamine (Example 35)

At 20° C., 250 mg (0.866 mmol) of 3-{[5-chloro-4-(cyclopropylamino)pyrimidin-2-yl]amino}-benzenecarbaldehyde and 937 mg (8.66 mmol) of 1,3-propanedithiol are initially charged in 5 ml of dioxane, and 1.352 g (9.524 mmol) of BF₃.Et₂O are added. The mixture is stirred at 20° C. for 16 hours. The reaction solution is then neutralized with 5 ml of NaHCO₃ solution and extracted with 20 ml of dichloromethane. The organic phase is dried over MgSO₄ and freed from the solvent under reduced pressure. The crude product is purified by column chromatography on alumina (cyclohexane/ethyl acetate). This gives 210 mg of the desired product (logP (pH 2.7): 2.44).

¹H NMR (400 MHz, DMSO-d) δ=9.12 (s, 1H), 8.13 (t, 1H), 7.91 (s, 1H), 7.65-7.56 (m, 1H), 7.19 (t, 1H), 7.03-6.90 (m, 2H), 5.27 (s, 1H), 2.90-2.80 (m, 2H), 2.16-2.08 (m, 1H), 1.75-1.60 (m, 1H), 0.92-0.85 (m, 2H), 0.69-0.62 (m, 2H)

### 5-Chloro-N⁴-cyclopropyl-N²-[3-(1,3-oxathiolan-2-yl)phenyl]pyrimidine-2,4-diamine (Example 46)

At 20° C., 150 mg (0.52 mmol) of 3-{[5-chloro-4-(cyclopropylamino)pyrimidin-2-yl]amino}benzenecarbaldehyde and 41 mg (0.52 mmol) of 2-mercaptoethanol are initially charged in 15 ml of dioxane, and 81 mg (0.571 mmol) of BF₃.Et₂O are added. The mixture is stirred at 20° C. for 16 hours. The reaction solution is then neutralized with saturated NaHCO₃ solution and extracted with 20 ml of dichloromethane. The organic phase is dried over MgSO₄ and freed from the solvent under reduced pressure. The crude product is purified by column chromatography on alumina (cyclohexane/ethyl acetate). This gives 53 mg of the desired product (logP (pH 2.7): 2.05).

¹H NMR (400 MHz, DMSO-d) δ=9.08 (s, 1H), 8.03 (s, 1H), 7.90 (s, 1H), 7.72-7.64 (m, 1H), 7.20 (t, 1H), 7.05-6.93 (m, 2H), 5.99 (s, 1H), 4.45-4.35 (m, 1H), 3.95-3.85 (m, 1H), 2.97-2.88 (m, 1H), 0.85-0.75 (m, 2H), 0.70-0.60 (m, 2H)

### Preparation of compounds of the formula (Ic) (Example 66)

A mixture of 200 mg (0.693 mmol) of 3-{[5-chloro-4-(cyclopropylamino)pyrimidin-2-yl]amino}-benzenecarbaldehyde, 7 ml of anhydrous n-butanol and 0.26 ml (1.039 mmol) of 4N hydrochloric acid in dioxane is heated at 80° C. for 16 hours. After cooling, the reaction solution is neutralized with 5 ml of NaHCO₃ solution and extracted with 20 ml of dichloromethane. The organic phase is dried over MgSO₄ and freed from the solvent under reduced pressure. The crude product is purified by column chromatography on alumina (RS30) using the mobile phase cyclohexane/ethyl acetate. This gives 216 mg of the desired product (logP (pH 2.7): 4.64).

¹H NMR (400 MHz, acetonitrile-d) d=7.88 (s, 2H), 7.70-7.62 (m, 1H), 7.51 (s, 1H), 7.25 (t, 1H), 7.02 (d, 1H), 7.87 (s, 1H), 5.38 (s, 1H), 3.60-3.40 (m, 4H), 2.91-2.84 (m, 1H), 1.60-1.50 (m, 4H), 1.45-1.30 (m, 4H), 0.95-0.82 (m, 8H), 0.67-0.60 (m, 2H)

### EXAMPLES

The compounds of the formula (I) listed in Table I below are also obtained by the methods given above.

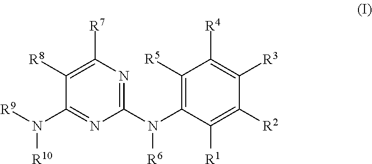

TABLE I

| Ex. | R¹ | R² | R³ | R⁴ | R⁵ | R⁶ | R⁷ | R⁸ | R⁹ | R¹⁰ | log p |
|---|---|---|---|---|---|---|---|---|---|---|---|
| 1 | H | 1,3-Dioxolan-2-yl | H | H | H | H | H | Chlorine | H | Cyclopropyl | 2.67[c]; 1.59[b] |
| 2 | H | 5,5-Dimethyl-1,3-dioxan-2-yl | H | H | H | H | H | Chlorine | H | Cyclopropyl | 2.45[b] |
| 3 | H | 4,5-Dimethyl-1,3-dioxolan-2-yl | H | H | H | H | H | Chlorine | H | Cyclopropyl | 2.2[b] |
| 4 | H | Bis(2-sulphanylethoxy)methyl | H | H | H | H | H | Chlorine | H | Cyclopropyl | 1.36[b]; 2.03[c] |
| 5 | H | 1,3-Dioxan-2-yl | H | H | H | H | H | Chlorine | H | Cyclopropyl | 2.83[c]; 1.69[b] |
| 6 | H | 2-Methyl-1,3-dioxan-2-yl | H | H | H | H | H | Chlorine | H | Cyclopropyl | 87.2[b] |
| 7 | H | 4-Ethyl-1,3-dioxolan-2-yl | H | H | H | H | H | Chlorine | H | Cyclopropyl | 3.48[c]; 2.29[b] |
| 8 | H | 4-Methyl-1,3-dioxolan-2-yl | H | H | H | H | H | Chlorine | H | Cyclopropyl | 1.9[b] |
| 9 | H | 4,6-Dimethyl-1,3-dioxan-2-yl | H | H | H | H | H | Chlorine | H | Cyclopropyl | 3.51[c]; 2.26[b] |
| 10 | H | 2-Methyl-1,3-dioxolan-2-yl | H | H | H | H | H | CF₃ | H | Cyclopropylmethyl | 3.16[a]; 3.79[c]; |
| | | | | | | | | | | | 3.15[b] |
| 11 | H | 2-Methyl-1,3-dioxolan-2-yl | H | H | H | H | H | CF₃ | H | Cyclopropyl | 3.46[c]; 3.03[b] |
| 12 | H | 2-Methyl-1,3-dioxolan-2-yl | H | H | H | H | H | Chlorine | H | Cyclopropyl | 3.09[c]; 1.9[b] |
| 13 | H | 1,3-Dioxolan-2-yl | H | H | H | H | H | Bromine | H | Cyclopropyl | 2.82[c]; 1.75[b] |
| 14 | H | 4,5-Dimethyl-1,3-dioxolan-2-yl | H | H | H | H | H | Bromine | H | Cyclopropyl | 3.5[c]; 2.31[b] |
| 15 | H | 1,3-Dioxan-2-yl | H | H | H | H | H | Bromine | H | Cyclopropyl | 2.97[c]; 96.9[b] |
| 16 | H | 5,5-Dimethyl-1,3-dioxan-2-yl | H | H | H | H | H | Bromine | H | Cyclopropyl | 3.9[c]; 2.65[b] |
| 17 | H | 4,5-Dimethyl-1,3-dioxolan-2-yl | H | H | H | H | H | Bromine | H | Cyclobutyl | 4.12[c]; 2.67[b] |
| 18 | H | 1,3-Dioxolan-2-yl | H | H | H | H | H | Chlorine | H | Cyclopropylmethyl | 3.06[c]; 1.73[b] |
| 19 | H | 5,5-Dimethyl-1,3-dioxan-2-yl | H | H | H | H | H | Chlorine | H | Cyclobutyl | 2.29[a]; 4.13[c]; |
| | | | | | | | | | | | 2.57[b] |
| 20 | H | 1,3-Dioxolan-2-yl | H | H | H | H | H | Bromine | | Cyclobutyl | 3.39[c]; 2.05[b] |
| 21 | H | 5,5-Dimethyl-1,3-dioxan-2-yl | H | H | H | H | H | Bromine | | Cyclobutyl | 4.51[c]; 3[b] |
| 22 | H | 1,3-Dioxolan-2-yl | H | H | H | H | H | CF₃ | H | Cyclobutyl | 3.57[c]; 3.06[b] |
| 23 | H | 1,3-Dioxolan-2-yl | H | H | H | H | H | Chlorine | CH₃ | Cyclopropyl | 3.45[c]; 2.52[b] |
| 24 | H | 1,3-Dioxolan-2-yl | H | H | H | H | H | CF₃ | H | Cyclopropyl | 3.02[c]; 3.7[b] |
| 25 | H | 5,5-Dimethyl-1,3-dioxan-2-yl | H | H | H | H | H | CF₃ | H | Cyclopropyl | 4.07[c]; 3.7[b] |
| 26 | H | 5,5-Dimethyl-1,3-dioxan-2-yl | H | H | H | H | H | CF₃ | H | Cyclobutyl | 4.62[c]; 4.22[b] |
| 27 | H | H | 2-Methyl-1,3- | H | H | H | H | Chlorine | H | Cyclopropyl | 2.99[c]; 1.84[b] |
| | | | dioxolan-2-yl | | | | | | | | |
| 28 | H | 4-(Methoxymethyl)-1,3-dioxolan-2-yl | H | H | H | H | H | Chlorine | H | Cyclopropyl | 2.81[c]; 2.21[b] |
| 29 | H | 4-Phenyl-1,3-dioxolan-2-yl | H | H | H | H | H | Chlorine | H | Cyclopropyl | 3.93[c] |
| 30 | H | 4-tert-Butyl-1,3-dioxolan-2-yl | H | H | H | H | H | Chlorine | H | Cyclopropyl | 4.34[c] |
| 31 | H | 1,3-Dioxolan-2-yl | H | H | H | H | H | Chlorine | H | Cyclobutyl | 3.23[c]; 1.91[b] |
| 32 | H | Tetrahydrofuro[3,4-d][1,3]dioxol-2-yl | H | H | H | H | H | Chlorine | H | Cyclopropyl | 1.55[b] |
| 33 | H | 4-(Chloromethyl)-1,3-dioxolan-2-yl | H | H | H | H | H | Chlorine | H | Cyclopropyl | 1.85[a]; 2.19[b] |
| 34 | H | 4-[(Prop-2-en-1-yloxy)methyl]-1,3- | H | H | H | H | H | Chlorine | H | Cyclopropyl | 3.39[c]; 2.25[b] |
| | | dioxolan-2-yl | | | | | | | | | |
| 35 | H | 1,3-Dithian-2-yl | H | H | H | H | H | Chlorine | H | Cyclopropyl | 3.65[c]; 2.44[b] |
| 36 | H | 4-[(4-Methoxyphenoxy)methyl]-1,3- | H | H | H | H | H | Chlorine | H | Cyclopropyl | 3.78[c]; 2.64[b] |
| | | dioxolan-2-yl | | | | | | | | | |
| 37 | H | 4-(2-Nitrophenyl)-1,3-dioxolan-2-yl | H | H | H | H | H | Chlorine | H | Cyclopropyl | 4[c]; 2.89[b] |
| 38 | H | 4-Hexyl-1,3-dioxolan-2-yl | H | H | H | H | H | Chlorine | H | Cyclopropyl | 5.29[c]; 4.14[b] |
| 39 | H | 4-Butyl-1,3-dioxolan-2-yl | H | H | H | H | H | Chlorine | H | Cyclopropyl | 4.4[c]; 3.16[b] |
| 40 | H | 4-[(Diethylamino)methyl]-1,3- | H | H | H | H | H | Chlorine | H | Cyclopropyl | 3.9[c]; 1.07[b] |
| | | dioxolan-2-yl | | | | | | | | | |
| 41 | H | 4-(Morpholin-4-ylmethyl)-1,3- | H | H | H | H | H | Chlorine | H | Cyclopropyl | 2.54[c] |
| | | dioxolan-2-yl | | | | | | | | | |
| 42 | H | 4-(Hydroxymethyl)-1,3-dioxolan-2-yl | H | H | H | H | H | Chlorine | H | Cyclopropyl | 1.96[c]; 1.16[b] |
| 43 | H | 4-(Pyrrolidin-1-ylmethyl)-1,3- | H | H | H | H | H | Chlorine | H | Cyclopropyl | 0.99[b] |
| | | dioxolan-2-yl | | | | | | | | | |
| 44 | H | Hexahydro-1,3-benzodioxol-2-yl | H | H | H | H | H | Chlorine | H | Cyclopropyl | 3.99[c]; 2.65[b] |
| 45 | H | 1,3-Dioxolan-2-yl | H | H | H | H | H | Chlorine | H | 2,2,2-Trifluoroethyl | 2.73[c]; 2.35[b] |
| 46 | H | 1,3-Oxathiolan-2-yl | H | H | H | H | H | Chlorine | H | Cyclopropyl | 3.26[c]; 2.05[b] |
| 47 | H | 1,3-Dithiolan-2-yl | H | H | H | H | H | Chlorine | H | Cyclopropyl | 3.68[c]; 2.42[b] |
| 48 | H | 2-Methyl-1,3-dioxan-2-yl | H | H | H | H | H | CF₃ | H | Cyclopropyl | 3.64[c]; 3.29[b] |
| 49 | H | 1,3-Dioxolan-2-yl | H | H | H | H | H | Iodine | H | Cyclopropyl | 3.02[c]; 1.75[b] |
| 50 | H | 1,3-Dioxolan-2-yl | Chlorine | H | H | H | H | Chlorine | H | Cyclopropyl | 3.28[c]; 2.31[b] |
| 51 | H | 1,3-Dioxan-2-yl | H | H | H | H | H | Fluorine | H | Cyclopropyl | 1.23[a]; 2.4[c]; |
| | | | | | | | | | | | 1.28[b] |
| 52 | H | 1,3-Dioxan-2-yl | H | H | H | H | H | Chlorine | H | 2,2-Difluoroethyl | 1.6[a]; 2.68[c]; |
| | | | | | | | | | | | 1.97[b] |
| 53 | H | 1,3-Dioxan-2-yl | H | H | H | H | H | Chlorine | H | 2,2,2-Trifluoroethyl | 2.08[a]; 2.86[c]; |
| | | | | | | | | | | | 2.48[b] |
| 54 | H | 1,3-Dioxan-2-yl | H | H | H | H | H | Chlorine | H | Prop-2-yn-1-yl | 1.38[a]; 2.48[c]; |
| | | | | | | | | | | | 1.67[b] |
| 55 | H | 1,3-Dioxan-2-yl | H | H | H | H | H | CH₃ | H | Cyclopropyl | 1.32[a]; 2.37[c]; |
| | | | | | | | | | | | 1.28[b] |
| 56 | H | 1,3-Dioxan-2-yl | H | H | H | H | H | Chlorine | H | 1-Methoxypropan-2-yl | 1.46[a]; 2.92[c]; |
| | | | | | | | | | | | 1.69[b] |
| 57 | H | 1,3-Dioxan-2-yl | H | H | H | H | H | Iodine | H | Cyclopropyl | 1.6[a]; 3.18[c]; |
| | | | | | | | | | | | 2.23[b] |
| 58 | H | 1,3-Dioxolan-2-yl | H | H | H | H | H | CH₃ | H | Cyclopropyl | 1.21[a]; 2.25[c]; |
| | | | | | | | | | | | 1.21[b] |
| 59 | H | 1,3-Dioxolan-2-yl | H | H | H | H | H | Fluorine | H | Cyclopropyl | 1.14[a]; 2.28[c]; |
| | | | | | | | | | | | 1.60[b] |
| 60 | H | 1,3-Dioxolan-2-yl | H | H | H | H | H | Chlorine | H | 2,2-Difluoroethyl | 1.49[a]; 2.55[c]; |
| | | | | | | | | | | | 1.88[b] |
| 61 | H | 1,3-Dioxolan-2-yl | H | H | H | H | H | Chlorine | H | 1-Methoxypropan-2-yl | 1.62[b] |
| 62 | H | 1,3-Dioxan-2-yl | Chlorine | H | H | H | H | Chlorine | H | Cyclopropyl | 3.47[c]; 2.44[b] |
| 63 | H | Dimethoxymethyl | H | H | H | H | H | Chlorine | H | Cyclopropyl | 3.07[c]; 1.87[b] |
| 64 | H | Diethoxymethyl | H | H | H | H | H | Chlorine | H | Cyclopropyl | 3.86[c]; 2.58[b] |
| 65 | H | Bis(propan-2-yloxy)methyl | H | H | H | H | H | Chlorine | H | Cyclopropyl | 4.44[c]; |
| 66 | H | Dibutoxymethyl | H | H | H | H | H | Chlorine | H | Cyclopropyl | 5.83[c]; 4.64[b] |
| 67 | H | 1,3-Dioxan-2-yl | H | H | H | H | H | Cl | H | 2-Ethylcyclopropyl | |
| 68 | H | 2-Methyl-1,3-dioxan-2-yl | H | H | H | H | H | CF₃ | H | Cyclopropylmethyl | 3.42[b] |
| 69 | H | 2-Methyl-1,3-dioxan-2-yl | H | H | H | H | H | CF₃ | H | Cyclobutyl | 4.24[c]; 3.81[b] |
| 70 | H | 4-(Methoxymethyl)-2-methyl-1,3- | H | H | H | H | H | Cl | H | Cyclopropyl | |
| | | dioxolan-2-yl | | | | | | | | | |
| 71 | H | 2,5,5-Trimethyl-1,3-dioxan-2-yl | H | H | H | H | H | Cl | H | Cyclopropyl | 4.35[c]; 2.99[b] |
| 72 | H | 2,4-Dimethyl-1,3-dioxolan-2-yl | H | H | H | H | H | CF₃ | H | Cyclopropyl | 3.94[c]; 3.52[b] |
| 73 | H | 2,5,5-Trimethyl-1,3-dioxan-2-yl | H | H | H | H | H | CF₃ | H | Cyclopropyl | 4.7[c]; 4.32[b] |
| 74 | H | 2-Ethyl-4-methyl-1,3-dioxolan-2-yl | H | H | H | H | H | Cl | H | Cyclopropyl | 4.1[c]; 2.67[b] |
| 75 | H | 1,3-Dioxolan-2-yl | H | H | H | H | H | Cl | H | 2-Methylcyclopropyl | 3.21[c] |
| 76 | H | 2-Methyl-1,3-dioxolan-2-yl | H | H | H | H | H | Cl | H | 2-Methylcyclopropyl | 3.67[c]; 2.32[b] |
| 77 | H | H | 1,3-Dioxan- | H | H | H | H | Br | H | Cyclobutyl | 3.61[c]; 2.2[b] |
| | | | 2-yl | | | | | | | | |
| 78 | H | H | 1,3-Dioxan- | H | H | H | H | CF₃ | H | Cyclopropyl | 3.21[c]; 2.72[b] |
| | | | 2-yl | | | | | | | | |
| 79 | H | 5-Cyclopropyl-1,3-dioxan-2-yl | H | H | H | H | H | Br | H | Cyclopropyl | 4.03[c]; 2.77[b] |
| 80 | H | 5-Cyclopropyl-1,3-dioxan-2-yl | H | H | H | H | H | Cl | H | Cyclobutyl | 4.43[c]; 2.93[b] |
| 81 | H | 5-Methylidene-1,3-dioxan-2-yl | H | H | H | H | H | Br | H | Cyclopropyl | 3.27[c]; 2.1[b] |
| 82 | H | 5-Methylidene-1,3-dioxan-2-yl | H | H | H | H | H | Br | H | Cyclobutyl | 3.85[c]; 2.43[b] |
| 83 | H | 4-Methyl-1,3-dioxolan-2-yl | H | H | H | H | H | CF₃ | H | Cyclopropyl | 2.44[a]; 3.45[c]; |
| | | | | | | | | | | | 2.94[b] |
| 84 | H | 4-Methyl-1,3-dioxolan-2-yl | H | H | H | H | H | Br | H | Cyclopropyl | 3.25[c]; 2.03[b] |
| 85 | H | 4-Methyl-1,3-dioxolan-2-yl | H | H | H | H | H | Br | H | Cyclobutyl | 3.84[c]; 2.39[b] |
| 86 | H | 4-Methyl-1,3-dioxolan-2-yl | H | H | H | H | H | Cl | H | Cyclobutyl | 3.68[c]; 2.2[b] |
| 87 | H | 4-Methyl-1,3-dioxolan-2-yl | H | H | H | H | H | CF₃ | H | Cyclobutyl | 4.04[c]; 3.51[b] |
| 88 | H | 4-Ethyl-1,3-dioxolan-2-yl | H | H | H | H | H | CF₃ | H | Cyclopropyl | 3.9[c]; 3.48[b] |
| 89 | H | 4-Ethyl-1,3-dioxolan-2-yl | H | H | H | H | H | Br | H | Cyclobutyl | 4.35[c]; 2.88[b] |
| 90 | H | 4-Ethyl-1,3-dioxolan-2-yl | H | H | H | H | H | Cl | H | Cyclobutyl | 4.18[c]; 2.65[b] |
| 91 | H | 5-Methylidene-1,3-dioxan-2-yl | H | H | H | H | H | Cl | H | Cyclobutyl | 3.73[c] |
| 92 | H | 5-Methylidene-1,3-dioxan-2-yl | H | H | H | H | H | Cl | H | Cyclopropyl | 3.18[c]; 2.01[b] |
| 93 | H | 5-Cyclopropyl-1,3-dioxan-2-yl | H | H | H | H | H | Cl | H | Cyclopropyl | 3.91[c]; 2.6[b] |
| 94 | H | 4-Phenyl-1,3-dioxan-2-yl | H | H | H | H | H | Cl | H | Cyclopropyl | 4.11[c]; 2.83[b] |
| 95 | H | 4-(tert-Butoxymethyl)-1,3-dioxolan-2-yl | H | H | H | H | H | Cl | H | Cyclopropyl | 4.08[c]; 2.76[b] |
| 96 | H | 4-(Propoxymethyl)-1,3-dioxolan-2-yl | H | H | H | H | H | Cl | H | Cyclopropyl | 3.79[c]; 2.51[b] |
| 97 | H | 4-Methyl-1,3-dioxan-2-yl | H | H | H | H | H | Cl | H | Cyclopropyl | 3.25[c]; 2[b] |
| 98 | H | 4,5,6-Trimethyl-1,3-dioxan-2-yl | H | H | H | H | H | Cl | H | Cyclopropyl | 4.21[c]; 2.77[b] |
| 99 | H | 5-Chloro-1,3-dioxan-2-yl | H | H | H | H | H | Cl | H | Cyclopropyl | 3.33[c]; 2.15[b] |
| 100 | H | 4,4-Dimethyl-1,3-dioxan-2-yl | H | H | H | H | H | Cl | H | Cyclopropyl | 3.57[c]; 2.22[b] |
| 101 | H | 5-Methyl-1,3-dioxan-2-yl | H | H | H | H | H | Cl | H | Cyclopropyl | 3.35[c]; 2.1[b] |
| 102 | H | 4,4,6,6-Tetramethyl-1,3-dioxan-2-yl | H | H | H | H | H | Cl | H | Cyclopropyl | 4.32[c]; 2.86[b] |
| 103 | H | 6,8-Dioxaspiro[3.5]non-7-yl | H | H | H | H | H | Cl | H | Cyclopropyl | 3.62[c]; 2.6[b] |
| 104 | H | 4-(Methoxymethyl)-1,3-dioxolan-2-yl | H | H | H | H | H | Br | H | Cyclopropyl | 3.02[c]; 1.9[b] |
| 105 | H | 4-(Methoxymethyl)-1,3-dioxolan-2-yl | H | H | H | H | H | CF₃ | H | Cyclopropyl | 2.26[a]; 3.23[c]; |
| | | | | | | | | | | | 2.74[b] |
| 106 | H | 4-(Methoxymethyl)-1,3-dioxolan-2-yl | H | H | H | H | H | CF₃ | H | Cyclobutyl | 3.77[c]; 3.22[b] |
| 107 | H | 4-[(Prop-2-en-1-yloxy)methyl]-1,3- | H | H | H | H | H | Br | H | Cyclopropyl | 2.01[a]; 2.43[b] |
| | | dioxolan-2-yl | | | | | | | | | |
| 108 | H | 4-[(Prop-2-en-1-yloxy)methyl]-1,3- | H | H | H | H | H | CF₃ | H | Cyclopropyl | 3.79[c]; 3.35[b] |
| | | dioxolan-2-yl | | | | | | | | | |
| 109 | H | 4-[(Prop-2-en-1-yloxy)methyl]-1,3- | H | H | H | H | H | CF₃ | H | Cyclobutyl | 4.35[c]; 3.84[b] |
| | | dioxolan-2-yl | | | | | | | | | |
| 110 | H | Tetrahydrofuro[3,4-d][1,3]dioxol-2-yl | H | H | H | H | H | Br | H | Cyclopropyl | 2.66[c] |
| 111 | H | Tetrahydrofuro[3,4-d][1,3]dioxol-2-yl | H | H | H | H | H | CF₃ | H | Cyclopropyl | 1.94[a]; 2.87[c] |
| 112 | H | Tetrahydrofuro[3,4-d][1,3]dioxol-2-yl | H | H | H | H | H | CF₃ | H | Cyclobutyl | 3.37[c]; 2.79[b] |
| 113 | H | 4-Ethyl-1,3-dioxolan-2-yl | H | H | H | H | H | CF₃ | H | Cyclobutyl | 4.56[c]; 4.06[b] |
| 114 | H | 4-(Methoxymethyl)-1,3-dioxolan-2-yl | H | H | H | H | H | Cl | H | Cyclobutyl | 3.42[c]; 2.09[b] |
| 115 | H | 4-(Methoxymethyl)-1,3-dioxolan-2-yl | H | H | H | H | H | Br | H | Cyclobutyl | 3.61[c]; 2.19[b] |
| 116 | H | 4-[(Prop-2-en-1-yloxy)methyl]-1,3- | H | H | H | H | H | Cl | H | Cyclobutyl | 4.07[c]; 2.59[b] |
| | | dioxolan-2-yl | | | | | | | | | |
| 117 | H | 4-[(Prop-2-en-1-yloxy)methyl]-1,3- | H | H | H | H | H | Br | H | Cyclobutyl | 4.24[c]; 2.77[b] |
| | | dioxolan-2-yl | | | | | | | | | |
| 118 | H | Tetrahydrofuro[3,4-d][1,3]dioxol-2-yl | H | H | H | H | H | Cl | H | Cyclobutyl | 3.04[c]; 1.77[b] |
| 119 | H | Tetrahydrofuro[3,4-d][1,3]dioxol-2-yl | H | H | H | H | H | Br | H | Cyclobutyl | 3.23[c]; 1.91[b] |
| 120 | H | 4-tert-Butyl-1,3-dioxan-2-yl | H | H | H | H | H | Br | H | Cyclopropyl | 4.7[c]; 3.34[b] |
| 121 | H | 4-tert-Butyl-1,3-dioxan-2-yl | H | H | H | H | H | Cl | H | Cyclopropyl | 3.16[b] |
| 122 | H | 4-tert-Butyl-1,3-dioxan-2-yl | H | H | H | H | H | CF₃ | H | Cyclopropyl | 4.82[c]; 4.44[b] |
| 123 | H | 5,7-Dioxaspiro[2.5]oct-6-yl | H | H | H | H | H | Cl | H | Cyclopropyl | 3.32[c]; 2.09[b] |
| 124 | H | 4-Methyl-6-(trifluoromethyl)-1,3- | H | H | H | H | H | Cl | H | Cyclopropyl | 2.84[b] |
| | | dioxan-2-yl | | | | | | | | | |
| 125 | H | 5-Ethoxy-1,3-dioxan-2-yl | H | H | H | H | H | Cl | H | Cyclopropyl | 3.27[c]; 2.07[b] |
| 126 | H | 1,3-Dioxepan-2-yl | H | H | H | H | H | Cl | H | Cyclopropyl | 3.48[c]; 2.14[b] |
| 127 | H | 1,3-Oxathian-2-yl | H | H | H | H | H | Cl | H | Cyclopropyl | 3.33[c]; 2.1[b] |
| 128 | H | 1,3-Dioxolan-2-yl | F | H | H | H | H | Cl | H | Cyclopropyl | 2.88[c]; 1.75[b] |
| 129 | H | 1,3-Dioxolan-2-yl | H | H | H | H | H | CF₃ | H | 2-Methylcyclopropyl | 3.58[c]; 3.03[b] |
| 130 | H | 4-(Methoxymethyl)-1,3-dioxolan-2-yl | H | H | H | H | H | CF₃ | H | 2-Methylcyclopropyl | 3.7[c]; 3.18[b] |
| 131 | H | 1,3-Dioxolan-2-yl | H | H | H | H | H | Br | H | 2-Methylcyclopropyl | 3.33[c] |
| 132 | H | 4-(Methoxymethyl)-1,3-dioxolan-2-yl | H | H | H | H | H | Br | H | 2-Methylcyclopropyl | 3.52[c]; 2.19[b] |
| 133 | H | 1,3-Dioxan-2-yl | H | H | H | H | H | Br | H | 2-Methylcyclopropyl | 3.53[c]; 2.18[b] |
| 134 | H | 1,3-Dioxolan-2-yl | H | H | H | H | H | Cl | H | Prop-2-yn-1-yl | 2.37[c]; 1.58[b] |
| 135 | H | H | 1,3-Dioxan- | H | H | H | H | Cl | H | Cyclopropyl | 2.82[c]; 1.72[b] |
| | | | 2-yl | | | | | | | | |
| 136 | H | 1,3-Dioxan-2-yl | Methoxy | H | H | H | H | CF₃ | H | Cyclopropyl | 3.18[c]; 2.38[b] |
| 137 | H | 1,3-Dioxolan-2-yl | Methoxy | H | H | H | H | Cl | H | Cyclopropyl | 2.57[c]; 1.42[b] |
| 138 | H | 1,3-Dioxolan-2-yl | Methoxy | H | H | H | H | Br | H | Cyclopropyl | 2.7[c]; 1.5[b] |
| 139 | H | 1,3-Dioxolan-2-yl | Methoxy | H | H | H | H | Br | H | Cyclobutyl | 3.29[c]; 1.79[b] |
| 140 | H | 4-(Methoxymethyl)-1,3-dioxan-2-yl | H | H | H | H | H | Cl | H | Cyclopropyl | |
| 141 | H | 1,3-Dioxolan-2-yl | F | H | H | H | H | Br | H | Cyclopropyl | 2.98[c]; 1.89[b] |
| 142 | H | 1,3-Dioxolan-2-yl | F | H | H | H | H | Br | H | Cyclobutyl | 3.55[c]; 2.22[b] |
| 143 | H | 1,3-Dioxolan-2-yl | F | H | H | H | H | CF₃ | H | Cyclopropyl | 3.2[c]; 2.76[b] |
| 144 | H | 1,3-Dioxan-2-yl | F | H | H | H | H | Cl | H | Cyclopropyl | 3.02[c]; 1.87[b] |
| 145 | H | 1,3-Dioxan-2-yl | F | H | H | H | H | Br | H | Cyclopropyl | 3.16[c]; 2.01[b] |
| 146 | H | 1,3-Dioxan-2-yl | F | H | H | H | H | Br | H | Cyclobutyl | 3.75[c]; 2.3[b] |
| 147 | H | 1,3-Dioxan-2-yl | F | H | H | H | H | CF₃ | H | Cyclopropyl | 3.40[c]; 2.27[b] |
| 148 | H | 1,3-Dioxolan-2-yl | H | H | H | H | H | Cl | H | Propan-2-yl | 3.04[c]; 1.7[b] |
| 149 | H | 1,3-Dioxan-2-yl | H | H | H | H | H | Cl | H | Propan-2-yl | 3.2[c]; 1.73[b] |
| 150 | H | 4-Ethyl-1,3-dioxolan-2-yl | H | H | H | H | H | Br | H | Cyclopropyl | 3.68[c] |
| 151 | H | 1,3-Dioxan-2-yl | H | H | H | H | H | Br | H | Cyclobutyl | 3.54[c]; 2.11[b] |
| 152 | H | 1,3-Dioxan-2-yl | H | H | H | H | H | Cl | H | Cyclopropylmethyl | 1.87[b] |
| 153 | H | 1,3-Dioxan-2-yl | H | H | H | H | H | CF₃ | H | Cyclopropyl | 2.71[b] |
| 154 | H | 4-[(2-Ethoxyethoxy)methyl]-1,3- | H | H | H | H | H | Cl | H | Cyclopropyl | 3.13[c]; 2.05[b] |
| | | dioxolan-2-yl | | | | | | | | | |
| 155 | H | 4-Methyl-1,3-dithiolan-2-yl | H | H | H | H | H | CF₃ | H | Cyclopropyl | 4.44[c]; 4.14[b] |
| 156 | H | 4-(Bromomethyl)-1,3-dioxolan-2-yl | H | H | H | H | H | Cl | H | Cyclopropyl | 3.48[c]; 2.39[b] |
| 157 | H | 4-Methyltetrahydro-3aH- | H | H | H | H | H | Cl | H | Cyclopropyl | 4.1[c]; 2.79[b] |
| | | cyclopenta[d][1,3]dioxol-2-yl | | | | | | | | | |
| 158 | H | 4,4,5,5-Tetramethyl-1,3-dioxolan-2-yl | H | H | H | H | H | Cl | H | Cyclopropyl | 4.13[c]; 2.78[b] |
| 159 | H | 5-Ethyl-5-(hydroxymethyl)-1,3- | H | H | H | H | H | Cl | H | Cyclopropyl | 2.77[c]; 1.82[b] |
| | | dioxan-2-yl | | | | | | | | | |
| 160 | H | 4-(Hydroxymethyl)-1,3-dithiolan-2-yl | H | H | H | H | H | Cl | H | Cyclopropyl | 2.72[c] |
| 161 | H | 4-Methyl-1,3-dithiolan-2-yl | H | H | H | H | H | Cl | H | Cyclopropyl | 4.14[c]; 2.79[b] |
| 162 | H | 4-(Hydroxymethyl)-1,3-dithiolan-2-yl | H | H | H | H | H | Cl | H | Cyclobutyl | 3.14[c]; 1.93[b] |
| 163 | H | 4-Methyl-1,3-dithiolan-2-yl | H | H | H | H | H | Cl | H | Cyclobutyl | 4.72[c]; 3.23[b] |
| 164 | H | 4-(Hydroxymethyl)-1,3-dithiolan-2-yl | H | H | H | H | H | Br | H | Cyclobutyl | 3.28[c]; 2.11[b] |
| 165 | H | 4-Methyl-1,3-dithiolan-2-yl | H | H | H | H | H | Br | H | Cyclobutyl | 4.92[c]; 3.49[b] |
| 166 | H | 4-(Hydroxymethyl)-1,3-dithiolan-2-yl | H | H | H | H | H | Br | H | Cyclopropyl | 2.85[c]; 1.89[b] |
| 167 | H | 4-Methyl-1,3-dithiolan-2-yl | H | H | H | H | H | Br | H | Cyclopropyl | 4.31[c]; 3.1[b] |
| 168 | H | 4-(Hydroxymethyl)-1,3-dithiolan-2-yl | H | H | H | H | H | CF₃ | H | Cyclopropyl | 3.03[c]; 2.65[b] |
| 169 | H | 4-(Methoxymethyl)-1,3-dioxan-2-yl | H | H | H | H | H | CF₃ | H | Cyclopropyl | |
| 170 | H | 5-(Methoxymethyl)-1,3-dioxan-2-yl | H | H | H | H | H | Cl | H | Cyclopropyl | |
| 171 | H | 5-(Methoxymethyl)-1,3-dioxan-2-yl | H | H | H | H | H | CF₃ | H | Cyclopropyl | |
| 172 | H | 5-Methoxy-1,3-dioxan-2-yl | H | H | H | H | H | Cl | H | Cyclopropyl | |
| 173 | H | 2-(Methoxymethyl)-1,3-dioxolan-2-yl | H | H | H | H | H | Cl | H | Cyclopropyl | |
| 174 | H | 5-Methoxy-1,3-dioxan-2-yl | H | H | H | H | H | CF₃ | H | Cyclopropyl | |

The logP values were measured in accordance with EEC Directive 79/831 Annex V.A8 by HPLC (High Performance Liquid Chromatography) on reversed-phase columns (C 18) using the methods below:

[a] The determination was carried out in the acidic range at pH 2.3 using the mobile phases 0.1% aqueous phosphoric acid and acetronitrile
linear gradient from 10% acetonitrile to 95% acetronitrile.
[b] The LC-MS determination in the acid range was carried out at pH 2.7 using the mobile phases 0.1% aqueous formic acid and acetonitrile (contains 0.1% formic acid)
linear gradient from 10% acetonitrile to 95% acetonitrile.
[c] The LC-MS determination in the neutral range was carried out at pH 7.8 using the mobile phases 0.001 molar aqueous ammonium bicarbonate solution and acetonitrile
linear gradient from 10% acetonitrile to 95% acetonitrile.

Calibration was carried out using unbranched alkan-2-ones (having 3 to 16 carbon atoms) with known logP values (determination of the logP values by the retention times using linear interpolation between two successive alkanones).

The lambda-maX values were determined in the maxima of the chromatographic signals using the UV spectra from 200 nm to 400 nm.

| Ex. No from | | |
|---|---|---|
| Table 1 | 1H NMR | M + 1 |
| 1 | ¹H NMR: δ = 9.07 (s, 1H), 8.02 (t, 1H), 7.91 (s, 1H), 7.77-7.21 | 333.1 |
| | (m, 1H), 7.23 (t, 1H), 7.03-6.93 (m, 2H), 5.67 (s, 1H), 4.03-3.89 | |
| | (m, 4H), 2.94-2.85 (m, 1H), 0.85-0.77 (m, 2H), 0.69-0.60 (m, 1H) | |
| 2 | ¹H NMR: δ = 9,94 (br. s, 1 H), 8.03 (s, 1 H), 7.90 (s, 1 H), 7.68 | 375.1 |
| | (dd, 1 H), 7.22 (t, 1 H), 6.98 (m, 2 H), 5.34 (s, 1 H), 3.61 (m, 4 | |
| | H), 2.92 (m, 1 H), 1.17 (s, 3 H) 0.78 (m, 2 H), 0.75 (s, 3 H), 0.64 | |
| | (m, 2 H). | |
| 3 | ¹H NMR: δ = 9.06 (s, 1 H), 7.98 (m, 1 H), 7.90 (s, 1 H), 7.75 (m, | 361.2 |
| | 1 H), 7.21 (m, 1 H), 6.95 (m, 2 H), 5.80 (s, 1 H), 3.74 (m, 1 H), | |
| | 3.67 (m, 1 H), 2.86 (m, 1 H), 1,27 (d, 3 H), 1.22 (sd, 3 H), 0.79 | |
| | (m, 2 H), 0.65 (m, 2 H). | |
| 4 | ¹H NMR (acetonitrile-d₃): δ = 5.09 (s, 1H) | 427.1 |
| 5 | ¹H NMR: δ = 9.04 (s, 1 H), 8.11 (s, 1 H), 7.90 (s, 1 H), 7.62 (dd 1 H), | 375.1 |
| | 7.18 (t, 1 H), 6.92-6.98 (m, 2 H), 4.07-4.11 (m, 6 H), 3.87- | |
| | 3.94 (m, 2 H), 1.90-1.98 (m, 1 H), 1.55-1.61 (m, 1 H), 1.40-1.44 | |
| | (m, 1 H), 0.79-0.84 (m, 1 H), 0.63-0.67 (m, 1 H). | |
| 6 | ¹H NMR: δ = 9.06 (s, br., 1H), 7.97-7.96 (m, 1H), 7.90 (s, 1H), | 361.1 |
| | 7.72-7.70 (m, 1H), 7.26 (t, 1H), 6.96 (s, br., 1H), 6.92-6.89 (m, | |
| | 1H), 3.79-3.70 (m, 4H), 2.95-2.89 (m, 1H), 1.93-1.84 (m, 1H), | |
| | 1.38 (s, 3H), 1.30-1.25 (m, 1H), 0.83-0.79 (m, 2H), 0.66-0.64 (m, | |
| | 2H) | |
| 7 | ¹H NMR (2 diastereomers): δ = [9.12, 9.05] (s, 1H), [8.02, 7.96] | 361.1 |
| | (t, 1H), 7.90 (s, 1H), 7.83-7.70 (1H), 7.26-7.19 (m, 1H), 7.05-6.94 | |
| | (2H), [5.78, 5.67] (s, 1H), 4.18-3.99 (m, 2H), 3.60-3.50 (m, 1H), | |
| | 2.93-2.83 (m, 1H), 1.70-1.48 (m, 2H), 0.97-0.89 (m, 3H), 0.83- | |
| | 0.75 (m, 2H), 0.66-0.61 (2H) | |
| 8 | ¹H NMR (2 diastereomers): δ = [9.08, 9.06] (s, 1H), [8.02, 7.97] | 347.1 |
| | (s, 1H), 7.90 (s, 1H), 7.80-7.70 (m, 1H), 7.27-7.19 (m, 1H), 7.02- | |
| | 6.93 (m, 2H), [5.81, 5.67] (s, 1H), 4.31-4.21 (m, 1H), [4.17, 4.04] | |
| | (dd, 1H), 3.46 (t, 1H), 2.93-2.85 (m, 1H), 1.30-1.20 (m, 3H), | |
| | 0.82-0.75 (m, 2H), 0.68-0.61 (m, 2H) | |
| 9 | ¹H NMR (2 diastereomers): δ = 5.73, 5.43 (s, 1H) | 375.1 |
| 10 | ¹H NMR: δ = 8.16 | 395.2 |
| 11 | ¹H NMR: δ = 9.45 (s, br., 1H), 8.17 (s, 1H), 8.03-8.02 (m, 1H), | 381.2 |
| | 7.77-7.74 (m, 1H), 7.23 (t, 1H), 7.03-7.01 (m, 1H), 6.79 (s, br., | |
| | 1H), 3.97-3.94 (m, 2H), 3.72-3.68 (m, 2H), 3.02-2.95 (m, 1H), | |
| | 1.53 (s, 3H), 0.85-0.80 (m, 2H), 0.69-0.66 (m, 2H) | |
| 12 | ¹H NMR: δ = 9.02 (s, br., 1H), 8.04-8.03 (m, 1H), 7.9 (s, 1H), | 347.2 |
| | 7.71-7.69 (m, 1H), 7.19 (t, 1H), 6.97-6.94 (m, 2H), 3.96-3.92 (m, | |
| | 2H), 3.71-3.68 (m, 2H), 2.98-2.91 (m, 1H), 1.53 (s, 3H), 0.83- | |
| | 0.76 (m, 2H), 0.67-0.65 (m, 2H) | |
| 13 | ¹H NMR: δ = 5.67 (s, 1H) | 378 |
| 14 | ¹H NMR (3 diastereomers): δ = 5.94, 5.81, 5.62 (s, 1H) | 406.1 |
| 15 | ¹H NMR: δ = 5.42 (s, 1H) | 392 |
| 16 | ¹H NMR: δ = 5.34 (s, 1H) | 420.1 |
| 17 | ¹H NMR (3 diastereomers): δ = 5.65, 5.84, 5.97 (s, 1H) | 420.1 |
| 18 | ¹H NMR: δ = 5.40 (s, 1H) | 347.1 |
| 19 | ¹H NMR: δ = 5.34 (s, 1H) | 389.1 |
| 20 | ¹H NMR: δ = 5.71 (s, 1H) | 392 |
| 21 | ¹H NMR: δ = 5.37 (s, 1H) | 434.1 |
| 22 | ¹H NMR: δ = 5.73 (s, 1H) | 381.1 |
| 23 | ¹H NMR: δ = 5.67 (s, 1H) | 347.1 |
| 24 | ¹H NMR: δ = 9.50 (s, 1H), 8.17 (s, 1H), 8.06 (s, 1H), 7.82-7.77 | 367.1 |
| | (m, 1H), 7.27 (t, 1H), 7.02 (d, 1H), 6.83 (s, 1H), 5.68 (s, 1H), | |
| | 4.02-3.89 (m, 4H), 2.98-2.88 (m, 1H), 0.86-0.79 (m, 2H), 0.69- | |
| | 0.62 (m, 2H) | |
| 25 | ¹H NMR: δ = 5.35 (s, 1H) | 409.2 |
| 26 | ¹H NMR: δ = 5.39 (s, 1H) | 423.2 |
| 27 | ¹H NMR: δ = 9.03 (s, br., 1H), 7.90 (s, 1H), 7.81-7.79 (d, 2H), | 347.1 |
| | 7.29-7.26 (m, 2H), 6.98 (s, br., 1H), 3.99-3.93 (m, 2H), 3.73-3.70 | |
| | (m, 2H), 2.89-2.83 (m, 1H), 1.54 (s, 3H), 0.83-0.76 (m, 2H), 0.68- | |
| | 0.66 (m, 2H) | |
| 28 | ¹H NMR (2 diastereomers): δ = [9.09, 9.08] (s, 1H), [8.04-7.96] | 377.1 |
| | (t, 1H), 7.81 (s, 1H), 7.83-7.70 (m, 1H), 7.28-7.18 (m, 1H), 7.03- | |
| | 6.93 (m, 2H), [5.78, 5.68] (s, 1H), 4.45-4.35 (m, 1H), [4.13, 4.01] | |
| | (dd, 1H), [3.74, 3.67] (dd, 1H), 3.55-3.42 (m, 2H), [3.34, 3.32] (s, | |
| | 3H), 2.94-2.85 (m, 1H), 0.85-0.75 (m, 2H), 0.69-0.62 (m, 2H) | |
| 29 | ¹H NMR (2 diastereomers): δ = 6.09, 5.89 (s, 1H) | 409.1 |
| 30 | ¹H NMR (2 diastereomers): δ = 5.80, 5.64 (s, 1H) | 389.1 |
| 31 | ¹H NMR: δ = 5.71 (s, 1H) | 347.1 |
| 32 | ¹H NMR: δ = 9.09 (s, 1H), 7.94 (t, 1H), 7.90 (s, 1H), 7.83-7.75 | 375.1 |
| | (m, 1H), 7.24 (t, 1H), 7.05-6.95 (m, 2H), 5.65 (m, 1H), 4.79 (m, | |
| | 2H), 3.94 (d, 2H), 3.58-3.49 (m, 2H), 2.93-2.84 (m, 1H), 0.82- | |
| | 0.74 (m, 2H), 0.67-0.60 (m, 2H) | |
| 33 | ¹H NMR (2 diastereomers): δ = 5.87; 5.73 (s, 1H) | 382.1 |
| 34 | ¹H NMR (2 diastereomers): δ = 5.78, 5.69 (s, 1H) | 403.1 |
| 35 | ¹H NMR: δ = 9.12 (s, 1H), 8.13 (t, 1H), 7.91 (s, 1H), 7.65-7.56 | 379.1 |
| | (m, 1H), 7.19 (t, 1H), 7.03-6.90 (m, 2H), 5.27 (s, 1H), 2.90-2.80 | |
| | (m, 2H), 2.16-2.08 (m, 1H), 1.75-1.60 (m, 1H), 0.92-0.85 (m, | |
| | 2H), 0.69-0.62 (m, 2H) | |
| 36 | ¹H NMR (2 diastereomers): δ = 5.86, 5.74 (s, 1H) | 469.1 |
| 37 | ¹H NMR (2 diastereomers): δ = 6.19, 5.91 (s, 1H) | 454.1 |
| 38 | ¹H NMR (2 diastereomers): δ = 5.78, 5.66 (s, 1H) | 417.2 |
| 39 | ¹H NMR (2 diastereomers): δ = 5.78, 5.66 (s, 1H) | 389.1 |
| 40 | ¹H NMR (2 diastereomers): δ = 9.08 (s, 1H), [8.01, 7.96] (s, | 418.1 |
| | 1H), 7.90 (s, 1H), 7.80-7.70 (m, 1H), 7.36-7.28 (m, 1H), 7.03- | |
| | 6.90 (m, 2H), [5.78, 5.67] (s, 1H), 4.20-3.98 (m, 2H), 3.72-3.60 | |
| | (m, 1H), 2.95-2.85 (m, 1H), 2.70-2.45 (m), 0.95 (q, 6H), 0.83- | |
| | 0.75 (m, 2H), 0.68-0.62 (m, 2H) | |
| 41 | ¹H NMR (2 diastereomers): δ = 5.78, 5.67 (s, 1H) | 432.2 |
| 42 | ¹H NMR (2 diastereomers): δ = 5.69, 5.46 (s, 1H) | 363.1 |
| 43 | ¹H NMR (2 diastereomers): δ = 5.76, 5.66 (s, 1H) | 416.1 |
| 44 | ¹H NMR: δ = 6.01 (s, 1H) | 387.1 |
| 45 | ¹H NMR: δ = 5.68 (s, 1H) | 375.1 |
| 46 | ¹H NMR: δ = 9.08 (s, 1H), 8.03 (s, 1H), 7.90 (s,1H), 7.72-7.64 | 349.1 |
| | (m, 1H), 7.20 (t, 1H), 7.05-6.93 (m, 2H), 5.99 (s, 1H), 4.45-4.35 | |
| | (m, 1H), 3.95-3.85 (m, 1H), 2.97-2.88 (m, 1H), 0.85-0.75 (m, | |
| | 2H), 0.70-0.60 (m, 2H) | |
| 47 | ¹H NMR: δ = 9.05 (s, 1H), 8.02 (t, 1H), 7.90 (s, 1H), 7.72-7.65 | 365.1 |
| | (m, 1H), 7.17 (t, 1H), 7.08-7.02 (m, 1H), 6.97 (s, 1H), 5.63 (s, | |
| | 1H), 3.50-3.27 (m, 4H), 3.0-2.91 (m, 1H), 0.87-0.72 (m, 2H), | |
| | 0.69-0.61 (m, 2H) | |
| 48 | ¹H NMR: δ = 9.48 (s, br., 1H), 8.17 (s, 1H), 8.00 (s, br., 1H), | 395.1 |
| | 7.79-7.76 (m, 1H), 7.30 (t, 1H), 6.98-6.96 (m, 1H), 6.78 (s, br., | |
| | 1H), 3.80-3.78 (m, 4H), 2.99-2.94 (m, 1H), 1.92-1.86 (m, 1H), | |
| | 1.39 (s, 3H), 1.31-1.26 (m, 1H), 0.86-0.81 (m, 2H), 0.69-0.65 (m, | |
| | 2H) | |
| 49 | ¹H NMR (acetonitrile-d₃): δ = 5.71 (s, 1H) | 425 |
| 50 | ¹H NMR (acetonitrile-d₃): δ = 8.00 (d, 1H), 7.89 (s, 1H), 7.87-7.81 | 367.1 |
| | (m, 1H), 7.60 (1H, s), 7.30 (d, 1H), 5.99 (s, 1H), 4.14-3.95 (m, | |
| | 4H), 2.93-2.85 (m, 1H), 0.88-0.78 (m, 2H), 0.68-0.63 (m, 2H) | |
| 51 | ¹H NMR (acetonitrile-d₃): δ = 5.46 (s, 1H) | 331.1 |
| 52 | ¹H NMR (acetonitrile-d₃): δ = 5.47 (s, 1H) | 371 |
| 53 | ¹H NMR (acetonitrile-d₃): δ = 5.47 (s, 1H) | 389 |
| 54 | ¹H NMR (acetonitrile-d₃): δ = 5.46 (s, 1H) | 345.1 |
| 55 | ¹H NMR (acetonitrile-d₃): δ = 5.45 (s, 1H) | 327.2 |
| 56 | ¹H NMR (acetonitrile-d₃): δ = 5.47 (s, 1H) | 379.1 |
| 57 | ¹H NMR (acetonitrile-d₃): δ = 5.46 (s, 1H) | 439 |
| 58 | ¹H NMR (acetonitrile-d₃): δ = 5.70 (s, 1H) | 313.1 |
| 59 | ¹H NMR (acetonitrile-d₃): δ = 5.71 (s, 1H) | 317.1 |
| 60 | ¹H NMR (acetonitrile-d₃): δ = 5.72 (s, 1H) | 357.1 |
| 61 | ¹H NMR (acetonitrile-d₃): δ = 5.72 (s, 1H) | 365.1 |
| 62 | ¹H NMR (acetonitrile-d₃): δ = 5.76 (s, 1H) | 382.1 |
| 63 | ¹H NMR: δ = 5.27 (s, 1H) | 335.1 |
| 64 | ¹H NMR: δ = 5.38 (s, 1H) | 363.1 |
| 65 | ¹H NMR (acetonitrile-d₃): δ = 7.93-7.85 (m, 2H), 7.70-7.62 (m, | 391.1 |
| | 1H), 7.51 (s, 1H), 7.24 (t, 1H), 7.01 (d, 1H), 5.87 (s, 1H), 5.42 (s, | |
| | 1H), 3.92-3.61 (m, 2H), 2.93-2.84 (m, 1H), 1.13 (dd, 12H), 0.92- | |
| | 0.80 (m, 2H), 0.67-0.58 (m, 2H) | |
| 66 | ¹H NMR (acetonitrile-d₃): δ = 7.88 (s, 2H), 7.70-7.62 (m, 1H), | 419.2 |
| | 7.51 (s, 1H), 7.25 (t, 1H), 7.02 (d, 1H), 7.87 (s, 1H), 5.38 (s, 1H), | |
| | 3.60-3.40 (m, 4H), 2.91-2.84 (m, 1H), 1.60-1.50 (m, 4H), 1.45- | |
| | 1.30 (m, 4H), 0.95-0.82 (m, 8H), 0.67-0.60 (m, 2H) | |
| 67 | ¹H NMR δ = 7.88 (s, 1 H), | 347.2 |
| 68 | ¹H NMR: δ = 8.16 | 409.1 |
| 69 | ¹H NMR: δ = 8.17 | 409.2 |
| 70 | ¹H NMR (diastereomer mixture): δ = 9.04 (s, br., 1H), 8.00-7.98 | 391.2 |
| | (m, 1H), 7.90 (s, 1H), 7.71-7.69 (m, 1H), 7.20 (t, 1H), 6.97-6.93 | |
| | (m, 1H), 4.14-3.67 (m, 3H), 3.46-3.40 (m, 2H), 3.30/3.22 (s, 3H), | |
| | 2.96-2.91 (m, 1H), 1.54/1.52 (s, 3H), 0.83-0.79 (m, 2H), 0.66- | |
| | 0.64 (m, 2H) | |
| 71 | ¹H NMR: δ = 7.90 | 389.2 |
| 72 | ¹H NMR (diastereomer mixture): δ = 9.45 (s, br., 1H), 8.16 (s, | 395.2 |
| | 1H), 8.04-8.02 (m, 1H), 7.78-7.72 (m, 1H), 7.22 (t, 1H), 7.06-7.00 | |
| | (m, 1H), 6.79 (s, br., 1H), 4.31-3.81 (m, 2H), 3.48-3.20 (m, 1H), | |
| | 3.01-2.96 (m, 1H), 1.54/1.51 (s, 3H), 1.23-1.21/1.13-1.11 (d, 3H), | |
| | 0.84-0.81 (m, 2H), 0.68-0.65 (m, 2H) | |
| 73 | ¹H NMR: δ = 8.17 | 423.2 |
| 74 | ¹H NMR: δ = 7.89 | 375.2 |
| 75 | ¹H NMR (6 stereoisomers, main isomer): δ = 9.07 (s, 1H), 7.89- | 347 |
| | 7.93 (m, 2H), 7.20-7.24 (m, 2H), 6.98 (d, 1H), 6.96 (s, 1H), | |
| | 5.68 (s, 1H), 3.92-4.03 (m, 4 H), 2.60-2.62 (m, 1H), 1.09 (d, 3H), | |
| | 0.93-1.01 (m, 1H), 0.79-0.83 (m, 1H), 0.55-0.59 (m, 1H). | |
| 76 | ¹H NMR (6 stereoisomers, main isomer): δ = 8.98 (s, 1 H), 7.89 | 361 |
| | (s, 1H), 7.85-7.86 (m, 1 H), 7.78-7.81 (m, 1 H), 7.17-7.20 (dd, | |
| | 1H), 6.95-6.97 (m, 1H), 6.89 (s, 1H), 3.93-3.97 (m, 2H), 3.69- | |
| | 3.73 (m, 2H), 2.63-2.67 (m, 1 H), 1.52 (s, 3 H), 1.10 (d, 3H), | |
| | 0.94-1.02 (m, 1H), 0.76-0.80 (m, 1 H), 0.58-0.62 (m, 1H). | |
| 77 | ¹H NMR: δ = 5.43 (s, 1H) | 406.1 |
| 78 | ¹H NMR: δ = 5.43 (s, 1H) | 381.2 |
| 79 | ¹H NMR: δ = 5.37 (s, 1H) | 432.1 |
| 80 | ¹H NMR: δ = 5.40 (s, 1H) | 401.2 |
| 81 | ¹H NMR: δ = 5.58 (s, 1H) | 404.1 |
| 82 | ¹H NMR: δ = 5.62 (s, 1H) | 418.1 |
| 83 | ¹H NMR (2 diastereomers): δ = 5.82, 5.67 (s, 1H) | 381.1 |
| 84 | ¹H NMR (2 diastereomers): δ = 5.81, 5.67 (s, 1H) | 392.1 |
| 85 | ¹H NMR (2 diastereomers): δ = 5.85, 5.71 (s, 1H) | 406 |
| 86 | ¹H NMR (2 diastereomers): δ = 5.85, 5.71 (s, 1H) | 361.1 |
| 87 | ¹H NMR (2 diastereomers): δ = 5.87, 5.73 (s, 1H) | 395.1 |
| 88 | ¹H NMR (2 diastereomers): δ = 5.80, 5.69 (s, 1H) | 395.1 |
| 89 | ¹H NMR (2 diastereomers): δ = 5.82, 5.70 (s, 1H) | 420.1 |
| 90 | ¹H NMR (2 diastereomers): δ = 5.84, 5.73 (s, 1H) | 375.1 |
| 91 | ¹H NMR: δ = 5.62 (s, 1H) | 373.1 |
| 92 | ¹H NMR: δ = 5.58 (s, 1H) | 359.1 |
| 93 | ¹H NMR: δ = 5.37 (s, 1H) | 387.1 |
| 94 | ¹H NMR: 8 = 5.68 (s, 1H) | 423.1 |
| 95 | ¹H NMR (2 diastereomers): δ = 5.78, 5.68 (s, 1H) | 419.2 |
| 96 | ¹H NMR (acetonitrile-d₃; 2 diastereomers): δ = 5.81, 5.72 (s, 1H) | 405.2 |
| 97 | ¹H NMR: δ = 9.04 (s, 1H), 8.09 (t, 1H), 7.90 (s, 1H), 7.66-7.60 | 361.2 |
| | (m, 1H), 7.18 (t, 1H), 6.99 (s, 1H), 6.94 (d, 1H), 5.42 (s, 1H), | |
| | 4.11-4.05 (m, 1H), 3.98-3.69 (m, 2H), 2.93-2.86 (m, 1H), 1.58- | |
| | 1.50 (m, 2H), 1.17 (d, 3H), 0.85-0.77 (m, 2H), 0.66-0.60 (m, 2H) | |
| 98 | ¹H NMR (3 diastereoisomers) d 5.47, 5.45, 5.42 (s, 1H) | 389.1 |
| 99 | ¹H NMR (2 diastereomers): δ = 5.87, 5.73 (s, 1H) | 382 |
| 100 | ¹H NMR: δ = 5.63 (s, 1H) | 375.2 |
| 101 | ¹H NMR: δ = 5.35 (s, 1H) | 361.1 |
| 102 | ¹H NMR: δ = 5.77 (s, 1H) | 403.2 |
| 103 | ¹H NMR: δ = 5.36 (s, 1H) | 387.1 |
| 104 | ¹H NMR (2 diastereomers): δ = [9.11, 9.10] (s, 1H), [8.13, 7.95] | 421.1 |
| | (t, 1H), 7.99 (s, 1H), 7.83-7.70 (m, 1H), 7.27-7.19 (m, 1H), 6.75 | |
| | (s, 1H), [5.78, 5.68] (s, 1H), 4.35-4.25 (m, 1H), [4.13, 4.01] (dd, | |
| | 1H), [3.74,3.67] (dd, 1H), 3.55-3.41 (m, 3H), [3.32, 3.30] (s, 3H), | |
| | 2.92-2.84 (m, 1H), 0.84-0.76 (m, 2H), 0.67-0.61 (m, 2H) | |
| 105 | ¹H NMR (2 diastereomers): δ = [9.51, 9.50] (s, 1H), 8.17 (s, | 411.2 |
| | 1H), [8.07, 7.99] ( t, 1H), 7.88-7.72 (m, 1H), 7.32-7.22 (m, 2H), | |
| | 7.09-7.00 (1H), 6.83 (s, 1H), [5.79, 5.70] (s, 1H), 4.35-4.25 (m, | |
| | 1H), [4.13, 4.02] (dd, 1H), [3.74, 3.68] (dd, 1H), 3.55-3.42 (m, | |
| | 2H), [3.34, 3.32] (s, 3H), 2.96-2.88 (m, 1H), 0.86-0.75 (m, 2H), | |
| | 0.68-0.63 (m, 2H) | |
| 106 | ¹H NMR (acetonitrile-d₃; 2 diastereomers): δ = 5.83, 5.74 (s, 1H) | 425.2 |
| 107 | ¹H NMR (acetonitrile-d₃; 2 diastereomers): δ = 5.78, 5.67 (s, 1H) | 448.1 |
| 108 | ¹H NMR (acetonitrile-d₃; 2 diastereomers): δ = 5.80, 5.70 (s, 1H) | 437.1 |
| 109 | ¹H NMR (acetonitrile-d₃; 2 diastereomers): δ = 5.82, 5.73 (s, 1H) | 451.1 |
| 110 | ¹H NMR: δ = 5.65 (s, 1H) | 420 |
| 111 | ¹H NMR (2 diastereomers): δ = 9.51 (s, 1H), 8.17 (s, 1H), [8.12, | 409.2 |
| | 7.97] (t, 1H), 7.89-7.70 (m, 1H), 7.32-7.22 (m, 1H), 7.11-6.98 (m, | |
| | 1H), 6.90-6.77 (m, 1H), [5.92, 5.67] (s, 1H), 4.86-4.77 (m, 2H), | |
| | 4.01-3.90 (m, 2H), 3.58-3.48 (m, 2H), 2.97-2.86 (m, 1H), 0.85- | |
| | 0.75 (m, 2H), 0.70-0.62 (m, 2H) | |
| 112 | ¹H NMR (2 diastereomers): δ = 5.97, 5.71 (s, 1H) | 423.2 |
| 113 | ¹H NMR (2 diastereomers): δ = 5.84, 5.72 (s, 1H) | 409.1 |
| 114 | ¹H NMR (2 diastereomers): δ = 5.81, 5.71 (s, 1H) | 391.1 |
| 115 | ¹H NMR (2 diastereomers): δ = 5.81, 5.72 (s, 1H) | 436.1 |
| 116 | ¹H NMR (2 diastereomers): δ = 5.82, 5.72 (s, 1H) | 417.1 |
| 117 | ¹H NMR (2 diastereomers): δ = 5.82, 5.72 (s, 1H) | |
| 118 | ¹H NMR: δ = 5.94 (s, 1H) | 389.1 |
| 119 | ¹H NMR: δ = 5.69 (s, 1H) | 434 |
| 120 | ¹H NMR: δ = 5.32 (s, 1H) | 448.1 |
| 121 | ¹H NMR: δ = 5.32 (s, 1H) | 403.2 |
| 122 | ¹H NMR: 8 = 5.34 (s, 1H) | 437.2 |
| 123 | ¹H NMR: δ = 5.51 (s, 1H) | 373.1 |
| 124 | ¹H NMR: δ = 5.63 (s, 1H) | 429.1 |
| 125 | ¹H NMR (acetonitrile-d₃; 2 diastereomers): δ = 7.91 (s, 1H), | 391.2 |
| | [7.85, 7.77] (t, 1H), 7.80-7.68 (m, 1H), 7.40 (d, 1H), 7.35-7.25 | |
| | (m, 1H), 7.12 (t, 1H), [5.94, 5.80] (s, 1H), 5.45 (s, 1H), 4.43-4.35 | |
| | (m, 1H), [4.20, 4.09] (dd, 1H), [3.92, 3.82] (dd, 1H), 3.66-3.47 (m, | |
| | 4H), 2.92-2.83 (m, 1H), 1.21 (q, 3H), 0.96-0.88 (m, 2H), 0.67- | |
| | 0.60 (m, 2H) | |
| 126 | ¹H NMR (DMF-d): δ = 9.30 (s, 1H), 8.24 (s, 1H), 7.94 (s, 1H), | 361.1 |
| | 7.90-7.83 (m, 1H), 7.32-7.21 (m, 2H), 7.03 (d, 1H), 5.66 (s, 1H), | |
| | 3.90-3.68 (m, 4H), 3.05-2.97 (m, 1H), 1.70-1.65 (m, 4H), 0.90- | |
| | 0.83 (m, 2H), 0.77-0.68 (m, 2H) | |
| 127 | ¹H NMR: δ = 9.08 (s, 1H), 8.14 (t, 1H), 7.90 (s, 1H), 7.62-7.56 | 363.1 |
| | (m, 1H), 7.18 (t, 1H), 7.02-6.96 (s, 1H), 6.91 (d, 1H), 5.79 (s, | |
| | 1H), 4.20-4.10 (m, 1H), 3.76 (td, 1H), 3.03-2.95 (m, 1H), 2.83- | |
| | 2.72 (m, 1H), 1.90-1.68 (m, 2H), 0.90-0.80 (m, 2H), | |
| 128 | ¹H NMR: δ = 9.09 (s, 1H), 8.01 (dd, 1H), 7.89 (s, 1H), 7.83-7.76 | 351.1 |
| | (m, 1H), 7.06 (dd, 1H), 6.99 (s, 1H), 5.91 (s, 1H), 4.05-3.90 (m, | |
| | 4H), 2.93-2.83 (m, 1H), 0.82-0.73 (m, 2H), 0.68-0.62 (m, 2H) | |
| 129 | ¹H NMR: δ = 5.68 (s, 1H) | 381.2 |
| 130 | ¹H NMR (2 diastereomers): δ = 5.78, 5.69 (s, 1H) | 425.2 |
| 131 | ¹H NMR (6 stereoisomers, main isomer, acetal-H): δ = 5.75 (s, 1H), | 391 |
| 132 | ¹H NMR (2 diastereomers): δ = 5.76, 5.67 (s, 1H) | 436.2 |
| 133 | ¹H NMR: δ = 5.43 (s, 1H) | 406.1 |
| 134 | ¹H NMR: δ = 5.70 (s, 1H) | 331 |
| 135 | ¹H NMR: δ = 9.06 (s, 1H), 7.90 (s, 1H), 7.80 (d, 2H), 7.25 (d, | 347.1 |
| | 2H), 7.00 (s, 1H), 5.41 (s, 1H), 4.15-4.08 (m, 2H), 3.95-3.85 (m, | |
| | 2H), 2.89-2.80 (m, 1H), 2.06-1.90 (m, 1H), 1.45-1.37 (m, 1H), | |
| | 0.80-0.73 (m, 2H), 0.68-0.62 (m, 2H) | |
| 136 | ¹H NMR: δ = 9.30 (s, 1H), 8.16 (s, 1H), 8.12 (s, 1H), 7.62 (dd, | 411.1 |
| | 1H), 6.91 (d, 1H), 6.75 (s, 1H), 5.73 (s, 1H), 4.10-4.00 (m, 2H), | |
| | 3.93-3.84 (m, 2H), 3.75 (s, 3H), 3.00-2.93 (m, 1H), 1.96-1.85 (m, | |
| | 1H), 1.45-1.35 (m, 1H), 0.88-0.80 (m, 2H), 0.67-0.60 (m, 2H) | |
| 137 | ¹H NMR: δ = 5.94 (s, 1H) | 363.1 |
| 138 | ¹H NMR: δ = 5.94 (s, 1H) | 408.1 |
| 139 | ¹H NMR: δ = 5.97 (s, 1H) | 422.1 |
| 141 | ¹H NMR: δ = 5.91 (s, 1H) | 396 |
| 142 | ¹H NMR: δ = 5.95 (s, 1H) | 410 |
| 143 | ¹H NMR: δ = 5.92 (s, 1H) | 385.1 |
| 144 | ¹H NMR: δ = 5.69 (s, 1H) | 365.1 |
| 145 | ¹H NMR: δ = 5.69 (s, 1H) | 410 |
| 146 | ¹H NMR: δ = 5.71 (s, 1H) | 424.1 |
| 147 | ¹H NMR: 8 = 5.70 (s, 1H) | 399.1 |
| 148 | ¹H NMR: δ = 9.01 (s, 1H), 7.95 (t, 1H), 7.90 (s, 1H), 7.61-7.55 | 335.1 |
| | (m, 1H), 7.22 (t, 1H), 6.95 (d, 1H), 6.45 (d, 1H), 5.68 (s, 1H), | |
| | 4.43-4.30 (m, 1H), 4.09-3.91 (m, 4H), 1.24 (d, 6H) | |
| 149 | ¹H NMR: δ = 5.44 (s, 1H) | 349.1 |
| 150 | ¹H NMR (2 diastereomers): δ = 5.78, 5.67 (s, 1H) | 406.1 |
| 151 | ¹H NMR (2 diastereomers): δ = 5.46, 5.69 (s, 1H) | 406 |
| 152 | ¹H NMR: δ = 5.44 (s, 1H) | 361.1 |
| 153 | ¹H NMR: δ = 5.44 (s, 1H) | 381.1 |
| 154 | ¹H NMR (2 diastereomers): δ = 5.78, 5.69 (s, 1H) | 435.1 |
| 155 | ¹H NMR (2 diastereomers): δ = [9.49, 9.46] (s, 1H), 8.17 (s, | 413 |
| | 1H), 8.05-8.00 (1H), 7.80-7.70 (m, 1H), 7.25-7.09 (m, 2H), 6.80 | |
| | (s, 1H), [5.73, 5.68] (s, 1H), [4.15-4.05, 3.95-3.85] (m, 1H), [3.53, | |
| | 3.41] (dd, 1H), 3.15-2.95 (m, 2H), [1.46, 1.40] (d, 3H), 0.85-0.76 | |
| | (m, 2H), 0.70-0.62 (m, 2H) | |
| 156 | ¹H NMR (2 diastereomers): δ = 5.88, 5.74 (s, 1H) | 426 |
| 157 | ¹H NMR (3 diastereoisomers) d 5.69, 5.88 (s, 1H) | 387.1 |
| 158 | ¹H NMR: δ = 5.44 (s, 1H) | 389 |
| 159 | ¹H NMR: δ = 5.34 (s, 1H) | 405.1 |
| 160 | ¹H NMR (2 diastereomers): δ = [9.07, 9.05] (s, 1H), 8.03-7.97 | 395.1 |
| | (m, 1H), 7.90 (s, 1H), 7.75-7.65 (m, 1H), 7.22-7.12 (m, 1H), 7.10- | |
| | 7.00 (m, 1H), 6.97 (s, 1H), 5.63 (s, 1H), [4.96, 4.92] (t, 1H), | |
| | 4.05-3.25 (m, 6H), 3.00-2.90 (m, 1H), 0.85-0.75 (m, 2H), 0.70- | |
| | 0.62 (m, 2H) | |
| 161 | ¹H NMR (2 diastereomers): δ = 5.72, 5.67 (s, 1H) | 379 |
| 162 | ¹H NMR (2 diastereomers): δ = 5.69, 5.67 (s, 1H) | 409.1 |
| 163 | ¹H NMR (2 diastereomers): δ = [9.01, 9.00] (s, 1H), 8.00-7.96 | 393 |
| | (m, 1H), 7.91 (s, 1H), 7.56-7.46 (m, 1H), 7.22-7.14 (m, 1H), 7.10- | |
| | 7.03 (t, 1H), 6.95 (d, 1H), [5.76, 5.70](s, 1H), 4.70-4.58 (m, 1H), | |
| | [4.18-4.09, 3.98-3.87] (m, 1H), [3.57, 3.42] (dd, 1H), 3.16-3.04 | |
| | (m, 1H), 2.36-2.25 (m, 2H), 2.20-2.06 (m, 2H), 1.78-1.60 (m, | |
| | 2H), [1.48, 1.42] (d, 3H), | |
| 164 | ¹H NMR (2 diastereomers): δ = 5.69, 5.67 (s, 1H) | 454 |
| 165 | ¹H NMR (2 diastereomers): δ = 5.76, 5.70 (s, 1H) | 438 |
| 166 | ¹H NMR (2 diastereomers): δ = 5.64 (s, 1H) | 440 |
| 167 | ¹H NMR (2 diastereomers): δ = 5.72, 5.67 (s, 1H) | 424 |
| 168 | ¹H NMR (2 diastereomers): 8 = 5.65 (s, 1H) | 429 |

The chemical NMR shifts in ppm were measured at 400 MHz, unless indicated otherwise in the solvent DMSO-d₆ using tetramethylsilane as internal standard.

The abbreviations below describe the signal splitting:

s=singlet, d=doublet, t=triplet, q=quadruplet, m=multiplet

### Example A

### Venturia Test (Apple)/Protective

Solvents: 24.5 parts by weight of acetone
24.5 parts by weight of dimethylacetamide
   Emulsifier: 1 part by weight of alkylaryl polyglycol ether

To produce a suitable preparation of active compound, 1 part by weight of active compound is mixed with the stated amounts of solvents and emulsifier, and the concentrate is diluted with water to the desired concentration.

To test for protective activity, young plants are sprayed with the preparation of active compound at the stated application rate. After the spray coating has dried on, the plants are inoculated with an aqueous conidia suspension of the apple pathogen Venturia inaequalis and then remain in an incubation cabinet at about 20° C. and 100% relative atmospheric humidity for 1 day.

The plants are then placed in a greenhouse at about 21° C. and a relative atmospheric humidity of about 90%.

Evaluation is carried out 10 days after the inoculation. 0% means an efficacy which corresponds to that of the control, whereas an efficacy of 100% means that no infection is observed.

In this test, the examples Nos. 1, 5, 6, 7, 8, 9, 10, 11, 12, 13, 14, 15, 16, 18, 20, 22, 23, 24, 44, 48, 51, 52, 53, 57, 59, 63, 64, 65, 67, 70, 75, 100, 104, 116, 118, 128, 141, 145, 151, 159, 160, 162 and 163 from Table I show, at an active compound concentration of 100 ppm, an efficacy of 70% or more.

### Example B

### Botrytis Test (Bean)/Protective

Solvents: 24.5 parts by weight of acetone
24.5 parts by weight of dimethylacetamide
   Emulsifier: 1 part by weight of alkylaryl polyglycol ether

To produce a suitable preparation of active compound, 1 part by weight of active compound is mixed with the stated amounts of solvents and emulsifier, and the concentrate is diluted with water to the desired concentration.

To test for protective activity, young plants are sprayed with the preparation of active compound at the stated application rate. After the spray coating has dried on, two small pieces of agar colonized by Botrytis cinerea are placed onto each leaf. The inoculated plants are placed in a dark chamber at about 20° C. and 100% relative atmospheric humidity.

The size of the infected areas on the leaves is evaluated 2 days after the inoculation. 0% means an efficacy which corresponds to that of the control, whereas an efficacy of 100% means that no infection is observed.

In this test, the examples Nos. 1, 2, 3, 5, 6, 7, 8, 9, 10, 11, 12, 13, 14, 15, 16, 18, 20, 22, 24, 44, 57, 63, 64, 67, 70, 75, 76, 100, 101, 104, 116, 118, 128, 141, 145, 151, 160 and 162 from Table I showed, at an active compound concentration of 100 ppm, an efficacy of 70% or more.

### Example C

### Sphaerotheca Test (Cucumber)/Protective

Solvent: 49 parts by weight of N,N-dimethylformamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young cucumber plants are sprayed with the preparation of active compound at the stated application rate. One day after the treatment, the plants are inoculated with a spore suspension of Sphaerotheca fuliginea. The plants are then placed in a greenhouse at 70% relative atmospheric humidity and a temperature of 23° C.

Evaluation is carried out 7 days after the inoculation. 0% means an efficacy which corresponds to that of the control, whereas an efficacy of 100% means that no infection is observed.

In this test, the examples Nos. 1, 3, 5, 6, 7, 8, 9, 10, 11, 12, 13, 14, 15, 18, 19, 20, 21, 22, 23, 24, 27, 28, 29, 30, 35, 38, 39, 42, 45, 46, 47, 48, 53, 56, 59, 60, 61, 63, 64, 70, 71, 72, 75, 76, 83, 84, 86, 87, 88, 90, 92, 95, 96, 97, 98, 99, 104, 105, 107, 108, 110, 111, 112, 114, 116, 117, 118, 119, 127, 128, 135, 139, 141, 143, 144, 148, 150, 153, 155, 156, 160, 161, 162, 163, 164, 165, 166, 167 and 168 from Table I showed, at an active compound concentration of 500 ppm, an efficacy of 70% or more.

### Example D

Leptosphaeria nodorum Test (Wheat)/Protective
Solvent: 49 parts by weight of N,N-dimethylformamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young wheat plants are sprayed with the preparation of active compound at the stated application rate. One day after the treatment, the plants are inoculated with an aqueous spore suspension of Leptosphaeria nodorum and then remain at 100% rel. atmospheric humidity and 22° C. for 48 h. The plants are then placed in a greenhouse at 90% rel. atmospheric humidity and a temperature of 22° C.

Evaluation is carried out 7-9 days after the inoculation. 0% means an efficacy which corresponds to that of the control, whereas an efficacy of 100% means that no infection is observed.

In this test, the examples Nos. 1, 2, 3, 5, 6, 7, 8, 9, 10, 11, 12, 13, 14, 15, 16, 17, 18, 19, 20, 21, 22, 23, 24, 27, 28, 30, 31, 32, 33, 34, 39, 40, 41, 42, 43, 44, 45, 46, 47, 48, 49, 51, 57, 59, 60, 61, 63, 64, 65, 67, 68, 69, 70, 71, 72, 73, 75, 76, 77, 78, 79, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 116, 117, 118, 119, 122, 124, 125, 126, 127, 128, 129, 135, 136, 137, 138, 139, 141, 142, 143, 144, 145, 146, 147, 148, 149, 150, 151, 152, 153, 154, 155, 157, 158, 159, 160, 161, 162, 163, 164, 165, 166, 167 and 168 from Table I showed, at an active compound concentration of 500 ppm, an efficacy of 70% or more.

### Example E

Septoria tritici Test (Wheat)/Protective
Solvent: 50 parts by weight of N,N-dimethylacetamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are sprayed with a spore suspension of Septoria tritici. The plants remain in an incubation cabin at 20° C. and 100% relative atmospheric humidity for 48 hours. The plants are then placed under a transparent hood at 15° C. and 100% relative atmospheric humidity.

The plants are placed in a greenhouse at a temperature of about 15° C. and a relative atmospheric humidity of 80%.

Evaluation is carried out 21 days after the inoculation. 0% means an efficacy which corresponds to that of the control, whereas an efficacy of 100% means that no infection is observed.

In this test, the examples Nos. 1, 5, 6, 7, 8, 9, 10, 11, 12, 13, 15, 18, 20, 22, 23, 24, 28, 31, 32, 34, 39, 44, 47, 48, 49, 57, 67, 68, 70, 75, 75, 76, 78, 81, 83, 84, 85, 87, 88, 89, 92, 97, 100, 101, 104, 105, 107, 108, 116, 117, 118, 125, 126, 128, 141, 143, 148, 149, 150, 153, 154, 156, 159, 160, 162, 163, 165 and 167 from Table I showed, at an active compound concentration of 500 ppm, an efficacy of 70% or more.

### Example F

### Pyricularia Test (Rice)/Protective

Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and a concentrate is diluted with water and the stated amount of emulsifier to the desired concentration.

To test for protective activity, young rice plants are sprayed with the preparation of active compound at the stated application rate. One day after the treatment, the plants are inoculated with an aqueous spore suspension of Pyricularia oryzae. The plants are then placed in a greenhouse at 100% relative atmospheric humidity and 25° C.

Evaluation is carried out 5 days after the inoculation. 0% means an efficacy which corresponds to that of the control, whereas an efficacy of 100% means that no infection is observed.

In this test, the examples Nos. 1, 2, 5, 6, 10, 11, 12, 13, 14, 15, 16, 17, 20, 24, 27, 28, 31, 32, 33, 34, 41, 42, 43, 44, 46, 47, 48, 49, 53, 57, 75, 78, 81, 82, 84, 88, 91, 92, 97, 98, 99, 104, 105, 111, 127, 135, 141, 153, 155, 156, 161 and 167 from Table I showed, at an active compound concentration of 250 ppm, an efficacy of 80% or more.

### Example G

### Rhizoctonia Test (Rice)/Protective

Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and a concentrate is diluted with water and the stated amount of emulsifier to the desired concentration.

To test for protective activity, young rice plants are sprayed with the preparation of active compound at the stated application rate. One day after the treatment, the plants are inoculated with hyphae of Rhizoctonia solani. The plants are then placed in a greenhouse at 100% relative atmospheric humidity and 25° C.

Evaluation is carried out 4 days after the inoculation. 0% means an efficacy which corresponds to that of the control, whereas an efficacy of 100% means that no infection is observed.

In this test, the examples Nos. 1, 2, 5, 6, 10, 11, 12, 14, 16, 17, 20, 24, 28, 31, 32, 33, 41, 42, 44, 46, 48, 75, 78, 81, 82, 91, 97, 104, 105, 111, 127, 135, 141, 153, 155 and 167 from Table I showed, at an active compound concentration of 250 ppm, an efficacy of 80% or more.

### Example H

### Cochliobolus Test (Rice)/Protective

Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. One day after the treatment, the plants are inoculated with an aqueous spore suspension of Cochliobolus miyabeanus. The plants are then placed in a greenhouse at 100% relative atmospheric humidity and 25° C.

Evaluation is carried out 4 days after the inoculation. 0% means an efficacy which corresponds to that of the control, whereas an efficacy of 100% means that no infection is observed.

In this test, the examples Nos. 1, 2, 5, 6, 10, 12, 13, 14, 15, 16, 17, 20, 24, 31, 32, 42, 44, 46, 47, 48, 49, 53, 57, 75, 78, 81, 84, 88, 91, 97, 98, 99, 104, 105, 111, 127, 135, 141, 153, 155, 156, 161 and 167 from Table I showed, at an active compound concentration of 250 ppm, an efficacy of 80% or more.

### Example I

### Gibberella Test (Rice)/Protective

Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. One day after the treatment, the plants are inoculated with an aqueous spore suspension of Gibberella zeae. The plants are then placed in a greenhouse at 100% relative atmospheric humidity and 25° C.

Evaluation is carried out 5 days after the inoculation. 0% means an efficacy which corresponds to that of the control, whereas an efficacy of 100% means that no infection is observed.

In this test, the compounds Nos. 84, 92 and 156 from Table I showed, at an active compound concentration of 250 ppm, an efficacy of 80% or more.

### Example J

### Phakopsora Test (Soya Beans)/Protective

Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young plants are sprayed with the preparation of active compound at the stated application rate. One day after the treatment, the plants are inoculated with an aqueous spore suspension of Phakopsora pachyrhizi. The plants are then placed in a greenhouse at 80% relative atmospheric humidity and 20° C.

Evaluation is carried out 1 day after the inoculation. 0% means an efficacy which corresponds to that of the control, whereas an efficacy of 100% means that no infection is observed.

In this test, the compound No. 105 from Table I showed, at an active compound concentration of 250 ppm, an efficacy of 80% or more.

### Example K

Production of Fumonisin FB1 by Fusarium proliferatum

The method used was adapted to microtitre plates using the method described by Lopez-Errasquin et al.: Journal of Microbiological Methods 68 (2007) 312-317.

Fumonisin-inducing liquid medium (Jiménez et al., Int. J. Food Microbiol. (2003), 89, 185-193) was inoculated with a concentrated spore suspension of Fusarium proliferatum (350 000 spores/ml, stored at −160° C.) to a final concentration of 2000 spores/ml.

The compounds were dissolved (10 mM in 100% DMSO) and diluted to 100 μM in H₂O. The compounds were tested at 7 concentrations in a range of from 50 μM to 0.01 μM (diluted, starting with the 100 μM stock solution in 10% DMSO).

From each diluted solution, 5 μl were mixed with 95 μl of inoculated medium in a well of a 96-well microarray plate. The plate was covered and incubated at 20° C. for 6 days.

At the beginning and after 6 days, an OD measurement (OD620 multiple read per well (square: 3×3)) was carried out to calculate the “pI50” growth.

After 6 days, a sample of the liquid medium was taken and diluted in 10% acetonitrile. The concentration of FB1 in the diluted samples was analysed by HPLC-MS/MS, and the results were used to calculate the “pI50 FB1” values.

HPLC-MS/MS was carried out using the paramters below:

Mass spectrometry instrument: Applied Biosystems API4000 QTrap

### HPLC: Agilent 1100

### Autosampler: CTC HTS PAL

Chromatography column: Waters Atlantis T3 (50×2 mm)
Examples of the Measured pI50 Values

Production of Fumonisin FB1 by Fusarium proliferatum

| Example No. | | |
|---|---|---|
| from Table I | pI50 Fum | pI50 growth |
| 1 | 6.8 | 6.4 |
| 5 | 6.6 | 6.1 |
| 6 | 6.1 | 5.5 |
| 7 | 6.3 | 5.8 |
| 8 | 6.3 | 5.9 |
| 9 | 6.2 | 5.8 |
| 11 | 5.7 | 5.1 |
| 12 | 6.3 | 5.8 |
| 13 | 6.6 | 6.2 |
| 14 | 5.9 | 5.5 |
| 15 | 6.3 | 5.9 |
| 16 | 6 | 5.4 |
| 17 | 5.9 | 5.2 |
| 18 | 5.8 | 5.4 |
| 20 | 6.2 | 5.7 |
| 22 | 5.8 | 5.5 |
| 23 | 5.6 | 4.9 |
| 24 | 6 | 5.5 |
| 27 | 6.4 | 6 |
| 28 | 6.5 | 5.9 |
| 30 | 6 | 5.3 |
| 104 | 6.6 | 5.8 |
| 105 | 6.2 | 5.5 |
| 97 | 6.3 | 5.6 |

### Example L

Production of DON/acetyl-DON by Fusarium graminearum

The compounds were tested in microtitre plates at 7 concentrations of from 0.07 μM to 50 μM in a DON-inducing liquid medium (1 g of (NH₄)₂HPO₄, 0.2 g of MgSO₄×7 H₂O, 3 g of KH₂PO₄, 10 g of glycerol, 5 g of NaCl and 40 g of sucrose per litre) with oat extract (10%) and DMSO (0.5%). Inoculation was carried out using a concentrated spore suspension of Fusarium graminearum at a final concentration of 2000 spores/ml.

The plate was incubated at high atmospheric humidity at 28° C. for 7 days.

At the beginning and after 3 days, an OD measurement was carried out at OD520 (repeated measurements: 3×3 measurements per well) to calculate the growth inhibition.

After 7 days, 100 μl of an 84/16 acetonitrile/water mixture were added, and samples of the liquid medium were then taken from each well and diluted 1:100 in 10% acetonitrile. The proportions of DON and acetyl-DON in the samples were analysed by HPLC-MS/MS, and the measured values were used to calculate the inhibition of the DON/AcDON production compared to an active compound-free control.

The HPLC-MS/MS measurements were carried out using the parameters below:

Ionization type: ESI negative
Ion spray voltage: −4500 V
Spray gas temperature: 500° C.
Decluster potential: −40 V
Collision energy: −22 eV

### Collision gas: N₂

NMR trace: 355.0>264.9;
HPLC column: Waters Atlantis T3 (trifunctional C18 bondung, capped)
Particle size: 3 μm
Column dimensions: 50×2 mm

### Temperature: 40° C.

Solvent A: water/2.5 mM NH₄OAc+0.05% CH₃COOH (v/v)
Solvent B: methanol/2.5 mM NH₄OAc+0.05% CH₃COOH (v/v)
Flow rate: 400 μl/minute
Injection volume: 11 μl

### Gradient:

| Time [min] | A % | B % |
|---|---|---|
| 0 | 100 | 0 |
| 0.75 | 100 | 0 |
| 1.5 | 5 | 95 |
| 4 | 5 | 95 |
| 5 | 100 | 0 |
| 10 | 100 | 0 |

### Example of DON Inhibition

The examples Nos. 1, 9, 17, 18, 22, 23, 30 and 97 showed an activity of >80% in the inhibition of DON/AcDON at 50 μM. The inhibition of growth of Fusarium graminearum by the examples having an activity >70% varied from 67 to 100% at 50 μM.

1. A compound of the formula (I), 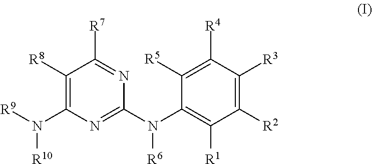
wherein
R¹ to R⁵ independently of one another represent hydrogen, OH, halogen, cyano, C₁-C₄-alkyl, C₁-C₄-alkoxy, C₁-C₄-haloalkyl, C₁-C₄-alkoxycarbonyl, NMe₂, SCF₃, SCH₃, OCF₂H or OCF₃,
where exactly one of the radicals R² and R³ represents a group of the formula E1, E2 or E3, 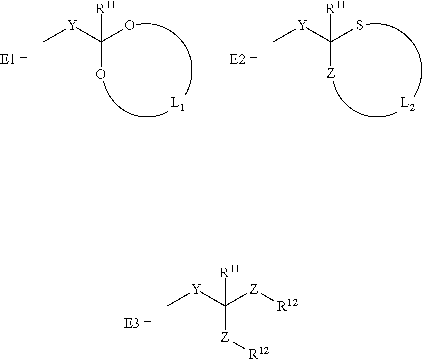
wherein:
Y represents a direct bond or a C₁-C₃-alkyl chain optionally substituted by C₁-C₄-alkyl, C₁-C₃-haloalkyl or C₁-C₃-alkoxyalkyl,
Z represents sulphur or oxygen,
with the proviso that, if R² or R³ represents a group E3, both Z represent either oxygen or sulphur,
L₁ represents an unsubstituted or substituted C₂-C₄-alkylene chain or CH₂CH═CHCH₂,
where the individual carbon atoms of the alkylene chain may carry one or more substituents, independently of one another selected from the list below:
hydrogen, OH, CH₂OH, halogen, CN, NMe₂, SCH₃, NO₂, optionally branched C₁-C₆-alkyl, optionally branched C₃-C₆-cycloalkyl, optionally branched C₁-C₄-alkoxy, optionally branched C₁-C₄alkoxycarbonyl, optionally branched C₁-C₄-alkoxy-C₁-C₄-alkyl, optionally substituted phenyl or benzyl, optionally branched C₁-C₄-haloalkyl, optionally branched C₁-C₄-alkylmercapto-C₁-C₄-alkyl, C₁-C₄-alkylcarbonyl, CH₂O(CO)CH₃, morpholin-4-ylmethyl, pyrrolidin-1-ylmethyl, (4-methoxyphenoxy)methyl, (diethylamino)methyl, (prop-2-en-1-yloxy)methyl, (2-ethoxyethoxy)methyl, CH₂CN, CH₂CH₂CN, CH₂SiMe₃, 2,2-difluorocyclopropyl or methylidene,
or
two substituents attached to a carbon atom together with this carbon atom form a 3- to 6-membered unsubstituted or substituted saturated carbocycle,
where the substituents independently of one another are selected from the list below:
hydrogen, fluorine, optionally branched C₁-C₄-alkyl, optionally branched C₁-C₄-alkoxy or optionally branched C₁-C₄-haloalkyl,
or
two substituents attached to two adjacent carbon atoms together with these two carbon atoms form
a 5- to 8-membered unsubstituted or substituted saturated carbocycle,
where the substituents independently of one another are selected from the list below:
hydrogen, fluorine, optionally branched C₁-C₄-alkyl, optionally branched C₁-C₄-alkoxy or optionally branched C₁-C₄-haloalkyl,
or
a 5- or 6-membered unsubstituted or substituted saturated heterocycle which contains an oxygen atom or a sulphur atom,
where the substituents independently of one another are selected from the list below:
hydrogen, fluorine, optionally branched C₁-C₄-alkyl, optionally branched C₁-C₄-alkoxy or optionally branched C₁-C₄-haloalkyl,
or
an unsubstituted or substituted phenyl ring,
where the substituents independently of one another are selected from the list below:
hydrogen, halogen, CN, SCH₃, NO₂, optionally branched C₁-C₄-alkyl, optionally branched C₁-C₄-alkoxy, optionally branched C₁-C₄-alkylcarbonyl, optionally branched C₁-C₄-haloalkyl or optionally branched C₁-C₄-haloalkoxy,
L₂ is an unsubstituted or substituted C₂- to C₃-alkylene chain,
where the individual carbon atoms may carry one or more substituents independently of one another selected from the list below:
hydrogen, CH₂OH, optionally branched C₁-C₄-alkyl, optionally branched C₁-C₄-alkoxy, optionally branched C₁-C₄-alkoxy-C₁-C₄-alkyl, optionally branched C₁-C₄-carbonyloxy-C₁-C₄-alkyl, optionally branched C₁-C₄-alkoxycarbonyl, branched C₁-C₄-haloalkyl, CH₂O(CO)CH₃, CH₂O(CO)CH₂CH₃, phenyl or benzyl,
R¹¹ represents hydrogen, CH₂OH, optionally branched C₁-C₄-alkyl, optionally branched C₁-C₄-alkoxy-C₁-C₄-alkyl, optionally branched C₁-C₄-haloalkyl, C₁-C₄-alkoxycarbonylmethyl, optionally branched C₃-C₆-cycloalkyl, CH₂CN, CH₂CH₂CN, (2-methyl-1,3-dioxan-2-yl)methyl, thiophen-2-yl, CH₂CH₂OH, phenyl or CH₂Ph,
R¹² represents branched or straight-chain C₁-C₅-alkyl, CH₂CH₂SH, prop-2-en-1-yl, COCH₃, COCH₂CH₃ or CH₂Ph,
R⁶ represents hydrogen, methyl, C₁-C₄-alkylcarbonyl, CHO, C₁-C₄-alkoxy-C₁-C₄-alkylcarbonyl, C₁-C₄-alkoxy-C₁-C₄-alkyl, optionally branched C₁-C₄-alkoxycarbonyl, C₁-C₄-haloalkylcarbonyl, C₁-C₃-alkenyl, C₁-C₃-alkynyl, C₁-C₄-alkylsulphinyl, C₁-C₄-alkylsulphonyl, optionally substituted benzyl, C₁-C₄-trialkylsilyl, C₁-C₄-trialkylsilylethyl or C₁-C₄-dialkylmonophenylsilyl,
where the substituents independently of one another are selected from the group consisting of hydrogen, methyl, fluorine, chlorine or bromine, C₁-C₄-alkyl, C₁-C₄-alkoxy, hydroxyl, C₁-C₄-haloalkyl and cyano,
R⁷ represents hydrogen, cyano, C₁-C₃-alkyl or C₁-C₃-haloalkyl,
R⁸ represents halogen, cyano, CF₃, methyl, CFH₂, CF₂H, CCl₃, SMe, S(O)Me or SO₂Me,
R⁹ represents hydrogen, straight-chain or branched C₁-C₃-alkyl, 2-methoxyethan-1-yl, prop-2-en-1-yl, C₁-C₄-alkoxy(C₁-C₄)alkyl, straight-chain or branched (C₁-C₄-alkyl)carbonyl, (C₁-C₄-haloalkyl)carbonyl, unsubstituted or substituted benzyl, C₁-C₆-trialkylsilyl, C₁-C₄-trialkylsilylethyl, C₁-C₄-dialkylmonophenylsilyl, (C₁-C₄-alkoxy)carbonyl, C₁-C₆-alkylsulphinyl, C₁-C₆-alkylsulphonyl, C₁-C₆-haloalkylsulphinyl or C₁-C₆-haloalkylsulphonyl,
where the substituents independently of one another are selected from the group consisting of hydrogen, halogen, nitro, C₁-C₄-alkyl, C₁-C₄-alkoxy, hydroxyl, C₁-C₄-haloalkyl and cyano,
R¹⁰ represents straight-chain or branched unsubstituted or substituted C₁-C₇-alkyl, straight-chain or branched unsubstituted or substituted C₂-C₇-haloalkyl, unsubstituted or substituted C₃-C₇-cycloalkyl, straight-chain or branched unsubstituted or substituted C₃-C₇-cycloalkyl(C₁-C₃)alkyl, straight-chain or branched unsubstituted or substituted C₃-C₇-alkenyl, straight-chain or branched unsubstituted or substituted C₃-C₇-alkynyl, straight-chain or branched unsubstituted or substituted C₁-C₄-alkoxy(C₁-C₄)alkyl, straight-chain or branched unsubstituted or substituted C₁-C₄-haloalkoxy(C₁-C₄)alkyl, 2-methyl-1-(methylsulphanyl)propan-2-yl or oxetan-3-yl,
or
R⁹ and R¹⁰ together with the nitrogen atom to which they are attached form an unsubstituted or substituted 3- to 7-membered saturated cycle which may contain up to one further heteroatom selected from the group consisting of oxygen, sulphur and nitrogen,
where the substituents R¹⁰ independently of one another are selected from the group consisting of methyl, ethyl, isopropyl, cyclopropyl, fluorine, chlorine and/or bromine atoms, methoxy, ethoxy, methylmercapto, ethylmercapto, cyano, hydroxyl and CF₃,
and agrochemically active salts thereof.

2. The compound of the formula (I) according to claim 1, wherein:
R¹ to R⁵ independently of one another represent hydrogen, OH, OCF₃, halogen, OMe, SCF₃, methyl, ethyl, CF₃, CF₂CF₃, CO₂Me, CO₂Et, SCH₃ or OCF₂H,
where exactly one of the radicals R² and R³ represents a group of the formula E1, E2 or E3 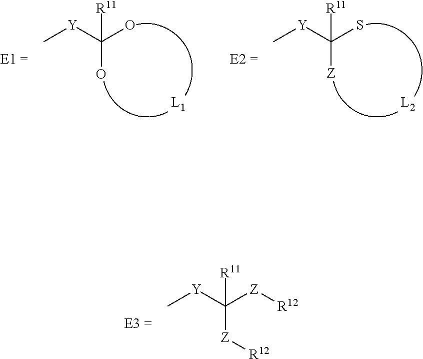
wherein:
Y represents a direct bond or —CH₂—, —CH₂CH₂—, —CHMe—, —CHEt-, —CH(CH₃)CH₂—, —CH₂CH(CH₃)— or —CH(OMe)—,
Z represents sulphur or oxygen,
with the proviso that, if R² or R³ represents a group E3, both Z represent either oxygen or sulphur,
L₁ represents an unsubstituted or substituted C₂- to C₃-alkylene, where the individual carbon atoms may carry one or more substituents independently of one another selected from the list below:
hydrogen, OH, methyl, ethyl, propyl, butyl, pentyl, hexyl, phenyl, isopropyl, tert-butyl, CH₂CH(CH₃)₂, CH₂C(CH₃)₃, 2-nitrophenyl, 2-chlorophenyl, 4-chlorophenyl, 4-methylphenyl, 4-aminophenyl, chloromethyl, CH₂CH₂Cl, CH₂F, CH₂Br, CH₂CH₂Br, hydroxymethyl, CH₂O(CO)CCH₃, methoxymethyl, morpholin-4-ylmethyl, pyrrolidin-1-ylmethyl, (4-methoxyphenoxy)methyl, (diethylamino)methyl, (prop-2-en-1-yloxy)methyl, CO₂Me, CO₂Et, COOPr, COOBu, CF₃, NO₂, CH₂SCH₃, CH₂SCH₂CH₃, CH₂CH₂SCH₃, CH₂CH₂SCH₂CH₃, CH₂CH₂OCH₃, CH₂OCH₂CH₃, CH₂CH₂OCH₂CH₃, CH₂OCH₂CH₂CH₃, (2-ethoxyethoxy)methyl, cyclopropyl, cyclobutyl, cyclopentyl, cyclohexyl, CH₂CN, CH₂CH₂CN, CH₂SiMe₃, 2,2-difluorocyclopropyl, CH₂OCMe₃, methylidene, chlorine, fluorine, bromine, iodine, OCH₃, OCH₂CH₃, OCH₂Ph, OC(CH₃)₃, OCH(CH₃)₂, O-propyl, O-butyl, COCH₃ or COCH₂CH₃,
or
two substituents attached to a carbon atom together with this carbon atom form a 3- to 6-membered unsubstituted or substituted saturated carbocycle,
where the substituents independently of one another are selected from the list below:
hydrogen, methyl, ethyl, propyl, isopropyl, tert-butyl, methoxy, ethoxy, propoxy, CF₃ or CHF₂,
or
two substituents attached to two adjacent carbon atoms together with these two carbon atoms form
a 5- to 8-membered unsubstituted or substituted saturated carbocycle,
where the substituents independently of one another are selected from the list below:
hydrogen, fluorine, methyl, ethyl, propyl, isopropyl, tert-butyl, methoxy, ethoxy, propoxy, CF₃ or CHF₂,
or
a 5- or 6-membered unsubstituted or substituted saturated heterocycle which contains one oxygen atom or one sulphur atom,
where the substituents independently of one another are selected from the list below:
hydrogen, fluorine, methyl, ethyl, propyl, isopropyl, tert-butyl, methoxy, ethoxy, propoxy, CF₃ or CHF₂,
or
an unsubstituted or substituted phenyl ring,
where the substituents independently of one another are selected from the list below:
hydrogen, chlorine, fluorine, CN, NO₂, methyl, ethyl, propyl, isopropyl, tert-butyl, methoxy, ethoxy, propoxy, CO—CH₃, COCH₂CH₃, CF₃, CHF₂, OCF₃ or OCHF₂,
L₂ is an unsubstituted or substituted C₂- to C₃-alkylene chain, where the individual carbon atoms may carry one or more substituents independently of one another selected from the list below:
hydrogen, methyl, ethyl, propyl, isopropyl, butyl, CH₂OH, OCH₂CH₃, methoxymethyl, CH₂OCH₂CH₃, CH₂O(CO)CCH₃, CH₂O(CO)CH₂CH₃, CH₂Br, CH₂C₁, CO₂Me, CO₂Et, phenyl or tert-butyl,
R¹¹ represents hydrogen, methyl, ethyl, propyl, isopropyl, tert-butyl, CH₂OH, CH₂OCH₃, CH₂OCH₂CH₃, CH₂CF₃, CH₂CN, CH₂CH₂CN, (2-methyl-1,3-dioxan-2-yl)methyl, CH₂Cl, CH₂Br, CH₂F, CHBrCH₃, CH₂CO₂CH₃, CH₂CO₂CH₂CH₃, CH₂COOPr, CH₂COOBu, thiophen-2-yl, cyclopropyl, cyclohexyl, CH₂CH₂OH, CH₂CH₂OCH₃, phenyl or CH₂Ph,
R¹² represents methyl, ethyl, propyl, butyl, isopropyl, CH₂CH(CH₃)₂, CH₂C(CH₃)₃, CH₂CH₂SH, prop-2-en-1-yl, COCH₃ or CH₂Ph,
R⁶ represents hydrogen, methyl, COMe, CHO, COCH₂OCH₃, CH₂OCH₃, COOMe or COOEt, COOtertBu, COOBn, COCF₃, CH₂CH═CH₂, CH₂C≡CH, SOCH₃, SO₂CH₃ or benzyl,
R⁷ represents hydrogen, cyano, methyl, CF₃ or CFH₂,
R⁸ represents halogen, cyano, CF₃, methyl, CFH₂, CF₂H, CCl₃, SMe, S(O)Me or SO₂Me,
R⁹ represents hydrogen, methyl, ethyl, propyl, propan-2-yl, 2-methoxyethan-1-yl, prop-2-en-1-yl, CH₂OCH₃, COMe, COOMe, COOEt, COOtertBu, COCF₃ or benzyl,
R¹⁰ represents straight-chain or branched unsubstituted or substituted C₁-C₆-alkyl, straight-chain or branched unsubstituted or substituted C₃-C₆-cycloalkyl-(C₁-C₂)alkyl, unsubstituted or substituted C₃-C₆-cycloalkyl, straight-chain or branched unsubstituted or substituted C₃-C₄-alkenyl, straight-chain or branched unsubstituted or substituted C₃-C₄-alkynyl, straight-chain or branched unsubstituted or substituted C₂-C₄-haloalkyl, straight-chain or branched unsubstituted or substituted C₁-C₂-alkoxy(C₁-C₄)alkyl, straight-chain or branched unsubstituted or substituted C₁-C₂-alkylmercapto(C₁-C₄)alkyl or oxetan-3-yl,
where the substituents in R¹⁰ independently of one another are selected from the group consisting of methyl, ethyl, isopropyl, cyclopropyl, fluorine, chlorine and/or bromine atoms, methoxy, ethoxy, methylmercapto, ethylmercapto, cyano, hydroxyl and CF₃,
or
R⁹ and R¹⁰ together with the nitrogen atom to which they are attached form an azetidinyl, pyrrolidinyl, piperidinyl, morpholinyl, azepanyl, 4-methylpiperazin-1-yl, 2-methylpiperidin-1-yl, -methylpyrrolidin-1-yl, 2-methylazetidin-1-yl or thiomorpholinyl ring,
and agrochemically active salts thereof.

3. The compound of the formula (I) according to claim 1,
wherein:
R¹ to R⁵ independently of one another represent hydrogen, OH, OCF₃, halogen, OMe, SCF₃, methyl, ethyl, CF₃, CF₂CF₃ or CO₂Me,
where exactly one of the radicals R² and R³ represents a group of the formula E1, E2 or E3, 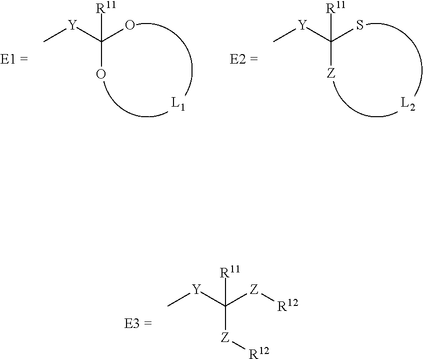
wherein:
Y represents a direct bond or —CH₂—, —CH₂CH₂— or —CHMe—,
Z represents sulphur or oxygen,
with the proviso that, if R² or R³ represents a group E3, both Z represent either oxygen or sulphur,
L₁ represents an unsubstituted or substituted C₂- to C₃-alkylene, where the individual carbon atoms may carry one or more substituents independently of one another selected from the list below:
hydrogen, methyl, ethyl, propyl, butyl, pentyl, hexyl, phenyl, isopropyl, tert-butyl, 2-nitrophenyl, 2-chlorophenyl, 4-chlorophenyl, 4-methylphenyl, 4-aminophenyl, chloromethyl, CH₂F, CH₂Br, hydroxymethyl, CH₂O(CO)CCH₃, methoxymethyl, morpholin-4-ylmethyl, pyrrolidin-1-ylmethyl, (4-methoxyphenoxy)methyl, (diethylamino)methyl, (prop-2-en-1-yloxy)methyl, CO₂Me, CO₂Et, CF₃, NO₂, CH₂SCH₃, CH₂SCH₂CH₃, CH₂CH₂SCH₃, CH₂CH₂SCH₂CH₃, CH₂CH₂OCH₃, CH₂OCH₂CH₃, CH₂CH₂OCH₂CH₃, CH₂OCH₂CH₂CH₃, (2-ethoxyethoxy)methyl, cyclopropyl, cyclopentyl, cyclohexyl, CH₂CN, CH₂CH₂CN, CH₂SiMe₃, 2,2-difluorocyclopropyl, CH₂OCMe₃, methylidene, chlorine, fluorine, bromine, iodine, OCH₃, OCH₂CH₃, OCH₂Ph or COCH₃,
or
two substituents attached to a carbon atom together with this carbon atom form a 3- to 6-membered unsubstituted or substituted saturated carbocycle,
where the substituents independently of one another are selected from the list below:
hydrogen, methyl, ethyl, propyl, methoxy, ethoxy or CF₃,
or
two substituents attached to two adjacent carbon atoms together with these two carbon atoms form
a 5- or 6-membered unsubstituted or substituted saturated carbocycle,
where the substituents independently of one another are selected from the list below:
hydrogen, fluorine, methyl, ethyl, propyl, methoxy, ethoxy or CF₃,
or
a 5- or 6-membered unsubstituted or substituted saturated heterocycle which contains an oxygen atom or a sulphur atom,
where the substituents independently of one another are selected from the list below:
hydrogen, fluorine, methyl, ethyl, tert-butyl, methoxy, ethoxy or CF₃,
or
an unsubstituted or substituted phenyl ring,
where the substituents independently of one another are selected from the list below:
hydrogen, chlorine, fluorine, CN, methyl, ethyl, Cert-butyl, methoxy, CF₃ or CO—CH₃,
L₂ represents an unsubstituted or substituted C₂- to C₃-alkylene, and
where the individual carbon atoms may carry one or more substituents independently of one another selected from the list below:
hydrogen, methyl, ethyl, propyl, isopropyl, butyl, hydroxymethyl, methoxymethyl, CH₂O(CO)CCH₃, CH₂Br, CO₂Me, phenyl or tert-butyl,
R¹¹ represents hydrogen, methyl, ethyl, propyl, isopropyl, tert-butyl, CH₂OCH₃, CH₂CF₃, CH₂CN, CH₂CH₂CN, (2-methyl-1,3-dioxan-2-yl)methyl, CH₂Cl, CH₂Br, CHBrCH₃, CH₂CO₂CH₃, CH₂CO₂CH₂CH₃, thiophen-2-yl or cyclopropyl,
R¹² represents methyl, ethyl, propyl, butyl, isopropyl, CH₂CH₂SH, prop-2-en-1-yl, COCH₃ or CH₂Ph,
R⁶ represents hydrogen, methyl, COMe, CHO or COCH₂OCH₃,
R⁷ represents hydrogen,
R⁸ represents halogen, cyano, CF₃, methyl, CFH₂, CF₂H, CCl₃, SMe, S(O)Me or SO₂Me,
R⁹ represents hydrogen, methyl, ethyl, propyl, propan-2-yl, 2-methoxyethan-1-yl or prop-2-en-1-yl,
R¹⁰ represents methyl, ethyl, propyl, cyclopropyl, cyclopropylmethyl, 1-cyclo-propyleth-1-yl, 2-methylcyclopropyl, 2,2-dimethylcyclopropyl, 2,2-dimethylprop-1-yl, tert-butyl, cyclobutyl, 2-butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, prop-2-yn-1-yl, butan-2-yl, 2,2,2-trifluoroethyl, 2,2-difluoroethyl, 2-methoxyethan-1-yl, 2-methylmercaptoethan-1-yl, 2-fluoroethan-1-yl, 2-chloroethan-1-yl, 2-cyanoethan-1-yl, 1-methoxypropan-2-yl, 3-methoxypropan-1-yl, 2-hydroxylethan-1-yl, 1-hydroxypropan-2-yl, 3-hydroxypropan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1-yl, 1-fluoropropan-2-yl, 2-methylcyclobut-1-yl or 3-methylcyclobut-1-yl,
or
R⁹ and R¹⁰ together with the nitrogen atom to which they are attached represent an azetidinyl, pyrrolidinyl, piperidinyl, morpholinyl, azepanyl, 4-methylpiperazin-1-yl, 2-methylpiperidin-1-yl, -methylpyrrolidin-1-yl, 2-methylazetidin-1-yl or thiomorpholinyl ring,
and agrochemically active salts thereof.

4. The compound of the formula (I) according to claim 1, wherein:
R¹ and R⁵ represent hydrogen,
R² represents hydrogen
or represents a group of the formula E1, E2 or E3 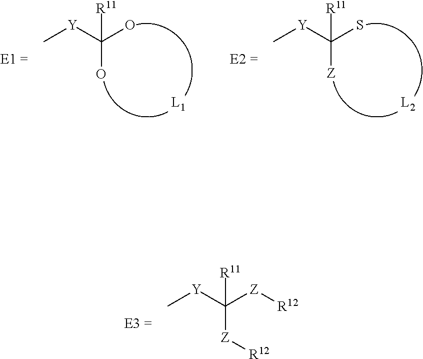
wherein:
Y represents a direct bond or —CH₂— or —CH₂CH₂—,
Z represents sulphur or oxygen,
with the proviso that, if R² or R³ represents a group E3, both Z represent either oxygen or sulphur,
L₁ represents an unsubstituted or substituted C₂- to C₃-alkylene, where the individual carbon atoms may carry one or more substituents independently of one another selected from the list below:
hydrogen, methyl, ethyl, propyl, butyl, pentyl, hexyl, phenyl, tert-butyl, 2-nitrophenyl, chloromethyl, CH₂F, hydroxymethyl, methoxymethyl, morpholin-4-ylmethyl, pyrrolidin-1-ylmethyl, (4-methoxyphenoxy)methyl, (diethylamno)methyl, (prop-2-en-1-yloxy)methyl, CO₂Me, CF₃, CH₂SCH₃, CH₂SCH₂CH₃, CH₂OCH₂CH₃, (2-ethoxyethoxy)methyl, cyclopropyl, CH₂CN, CH₂SiMe₃, 2,2-difluorocyclopropyl, CH₂OCMe₃, methylidene, chlorine, fluorine, OCH₃, OCH₂CH₃, OCH₂Ph or COCH₃,
or
two substituents attached to a carbon atom together with this carbon atom form a 5- to 6-membered unsubstituted saturated carbocycle,
or
two substituents attached to two adjacent carbon atoms together with these two carbon atoms form
a 5- or 6-membered unsubstituted saturated carbocycle,
or
a 5- or 6-membered unsubstituted saturated heterocycle which contains one oxygen atom,
or
an unsubstituted phenyl ring,
L₂ represents an unsubstituted or substituted C₂- to C₃-alkylene chain, where the individual carbon atoms may carry one or more substituents independently of one another selected from the list below:
hydrogen, methyl, ethyl or propyl,
R³ represents hydrogen, halogen, OMe or methyl,
or represents a group of the formula E1, E2 or E3, 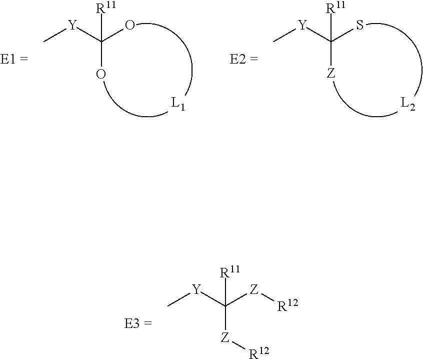
wherein:
Y represents a direct bond or —CH₂— or —CH₂CH₂—,
Z represents sulphur or oxygen,
with the proviso that, if R² or R³ represents a group E3, both Z either represent oxygen or represent sulphur,
L₁ represents an unsubstituted or substituted C₂- to C₃-alkylene, where the individual carbon atoms may carry one or more substituents independently of one another selected from the list below:
methyl, ethyl or methoxymethyl,
or
two substituents attached to a carbon atom together with this carbon atom form a 5- to 6-membered unsubstituted saturated carbocycle,
or
two substituents attached to two adjacent carbon atoms together with these two carbon atoms form
a 5- or 6-membered unsubstituted saturated carbocycle
or
a 5- or 6-membered unsubstituted saturated heterocycle which contains an oxygen atom
or
an unsubstituted phenyl ring,
L₂ represents an unsubstituted or substituted C₂- to C₃-alkylene, where the individual carbon atoms may carry one or more substituents independently of one another selected from the list below:
hydrogen, methyl, ethyl or propyl,
R¹¹ represents hydrogen, methyl, ethyl, propyl, CH₂OCH₃, CH₂CF₃, CH₂CN or (2-methyl-1,3-dioxan-2-yl)methyl,
R¹² represents methyl, ethyl, propyl, butyl, isopropyl or CH₂CH₂SH,
where R² and R³ are not both hydrogen,
with the proviso that, if R² is not hydrogen,
R³ can have only one of the meanings below:
hydrogen, chlorine, bromine, fluorine, iodine, OMe or methyl,
R⁴ represents hydrogen, halogen, OMe, SCF₃, methyl, ethyl, CF₃, CF₂CF₃ or CO₂Me,
R⁶ represents hydrogen,
R⁷ represents hydrogen,
R⁸ represents chlorine, bromine, fluorine, iodine, cyano, CF₃, methyl, SMe, S(O)Me or SO₂Me,
R⁹ represents hydrogen, methyl, ethyl, propyl or propan-2-yl,
R¹⁰ represents methyl, cyclopropyl, cyclopropylmethyl, 1-cyclopropyleth-1-yl, 2-methylcyclopropyl, cyclobutyl, 1-fluoroprop-2-yl, cyclopentyl, propan-2-yl, pentyl, prop-2-en-1-yl, prop-2-yn-1-yl, 2,2,2-trifluoroethyl, 2,2-difluoroethyl, 2-methoxyethan-1-yl, 2-fluorethan-1-yl, 2-chlorethan-1-yl, 1-methoxypropan-2-yl or 2-hydroxyethan-1-yl,
or
R⁹ and R¹⁰ together with the nitrogen atom to which they are attached form an azetidinyl, pyrrolidinyl, piperidinyl, morpholinyl, azepanyl, or thiomorpholinyl ring,
and agrochemically active salts thereof.

5. The compound of the formula (I) according to claim 1,
wherein:
R¹ and R⁵ represent hydrogen,
R² represents hydrogen, 1,3-dioxolan-2-yl, 4-methyl-1,3-dioxolan-2-yl, 4-ethyl-1,3-dioxolan-2-yl, 4-propyl-1,3-dioxolan-2-yl, 4-butyl-1,3-dioxolan-2-yl, 4-pentyl-1,3-dioxolan-2-yl, 4-hexyl-1,3-dioxolan-2-yl, 4-phenyl-1,3-dioxolan-2-yl, 4-tert-butyl-1,3-dioxolan-2-yl, 4-(2-nitrophenyl)-1,3-dioxolan-2-yl, 4-(chloromethyl)-1,3-dioxolan-2-yl, 4-(fluoromethyl)-1,3-dioxolan-2-yl, 4-(hydroxymethyl)-1,3-dioxolan-2-yl, 4-(methoxymethyl)-1,3-dioxolan-2-yl, 4-(morpholin-4-ylmethyl)-1,3-dioxolan-2-yl, 4-(pyrrolidin-1-ylmethyl)-1,3-dioxolan-2-yl, 4,5-dimethyl-1,3-dioxolan-2-yl, 4-[(4-methoxyphenoxy)methyl]-1,3-dioxolan-2-yl, 4-[(diethylamino)methyl]-1,3-dioxolan-2-yl, 4-[(prop-2-en-1-yloxy)methyl]-1,3-dioxolan-2-yl, hexahydro-1,3-benzodioxol-2-yl, tetrahydrofuro[3,4-d][1,3]dioxol-2-yl, (4R,5R)-4,5-bis(methoxycarbonyl)-1,3-dioxolan-2-yl, 4-(trifluoromethyl)-1,3-dioxolan-2-yl, 4-[(methylsulphanyl)methyl]-1,3-dioxolan-2-yl, 4-[(ethylsulphanyl)methyl]-1,3-dioxolan-2-yl, 4-(ethoxymethyl)-1,3-dioxolan-2-yl, 4,4,5,5-tetramethyl-1,3-dioxolan-2-yl, 1,3-benzodioxol-2-yl, 4-ethyl-4-methyl-1,3-dioxolan-2-yl, 4-[(2-ethoxyethoxy)methyl]-1,3-dioxolan-2-yl, (4R)-4-cyclopropyl-1,3-dioxolan-2-yl, (4R)-4-(cyanomethyl)-1,3-dioxolan-2-yl, 4-[(trimethylsilyl)methyl]-1,3-dioxolan-2-yl, 4-(2,2-difluorocyclopropyl)-1,3-dioxolan-2-yl, 4-(tert-butoxymethyl)-1,3-dioxolan-2-yl,
bis(2-sulphanylethoxy)methyl, bis(propan-2-yloxy)methyl, diethoxymethyl, dipropoxymethyl, diethoxymethyl, dimethoxymethyl,
1,3-dioxolan-2-ylmethyl, 2-(1,3-dioxolan-2-yl)ethyl,
1,3-dioxan-2-yl, 2-methyl-1,3-dioxan-2-yl, 4,6-dimethyl-1,3-dioxan-2-yl, 5,5-dimethyl-1,3-dioxan-2-yl, 4-methyl-1,3-dioxan-2-yl, 5-methyl-1,3-dioxan-2-yl, 4,4-dimethyl-1,3-dioxan-2-yl, 4-phenyl-1,3-dioxan-2-yl, 4,6-dimethyl-1,3-dioxan-2-yl, 4,4,6-trimethyl-1,3-dioxan-2-yl, 4,5,6-trimethyl-1,3-dioxan-2-yl, 4,4,6,6-tetramethyl-1,3-dioxan-2-yl, 4-tert-butyl-1,3-dioxan-2-yl, 4-(trifluoromethyl)-1,3-dioxan-2-yl, 5-methylidene-1,3-dioxan-2-yl, 5-chloro-1,3-dioxan-2-yl, 5,5-difluoro-1,3-dioxan-2-yl, 5-methoxy-1,3-dioxan-2-yl, 5-ethoxy-1,3-dioxan-2-yl, 5-(benzyloxy)-1,3-dioxan-2-yl, 5-cyclopropyl-1,3-dioxan-2-yl, 5-tert-butyl-1,3-dioxan-2-yl, 5,7-dioxaspiro[2.5]oct-6-yl, 6,8-dioxaspiro[3.5]non-7-yl, 5-oxo-1,3-dioxan-2-yl, 5-acetyl-4,6-dimethyl-1,3-dioxan-2-yl, 5-acetyl-4,5,6-trimethyl-1,3-dioxan-2-yl,
1,3-dioxan-2-ylmethyl, 2-(1,3-dioxan-2-yl)ethyl,
1,3-dithiolan-2-yl, 4-methyl-1,3-dithiolan-2-yl, 4-ethyl-1,3-dithiolan-2-yl, 4,5-dimethyl-1,3-dithiolan-2-yl, 1,3-dithian-2-yl, 4-methyl-1,3-dithian-2-yl, 1,3-oxathiolan-2-yl, 5-methyl-1,3-oxathiolan-2-yl, 4,5-dimethyl-1,3-oxathiolan-2-yl-1,3-oxathian-2-yl, 4-propyl-1,3-oxathian-2-yl,
bis(methylsulphanyl)methyl, bis(ethylsulphanyl)methyl,
2-methyl-1,3-dioxolan-2-yl, 2-ethyl-1,3-dioxolan-2-yl, 2-propyl-1,3-dioxolan-2-yl, 2-(methoxymethyl)-1,3-dioxolan-2-yl, 2-(2,2,2-trifluoroethyl)-1,3-dioxolan-2-yl, 2-[(2-methyl-1,3-dioxolan-2-yl)methyl]-1,3-dioxolan-2-yl,
2,4-dimethyl-1,3-dioxolan-2-yl, 4-ethyl-2-methyl-1,3-dioxolan-2-yl, 2,4,5-tri-methyl-1,3-dioxolan-2-yl, 4-(methoxymethyl)-2-methyl-1,3-dioxolan-2-yl, 2-methyltetrahydrofuro[3,4-d][1,3]dioxol-2-yl,
(2-methyl-1,3-dioxolan-2-yl)methyl, 2-(2-methyl-1,3-dioxolan-2-yl)ethyl,
2-methyl-1,3-dioxan-2-yl, 2-ethyl-1,3-dioxan-2-yl, 2-propyl-1,3-dioxan-2-yl, 2-(methoxymethyl)-1,3-dioxan-2-yl, 2-(2,2,2-trifluoroethyl)-1,3-dioxan-2-yl, 2-[(2-methyl-1,3-dioxan-2-yl)methyl]-1,3-dioxan-2-yl, 2,4-dimethyl-1,3-dioxan-2-yl, 2,4,6-trimethyl-1,3-dioxan-2-yl, 2,5,5-trimethyl-1,3-dioxan-2-yl,
1,1-dimethoxyethyl, 1,1-diethoxyethyl,
(2-methyl-1,3-dioxan-2-yl)methyl, 2-(2-methyl-1,3-dioxan-2-yl)ethyl, 2-methyl-1,3-dithiolan-2-yl, 2-methyl-1,3-oxathiolan-2-yl, 2-methyl-1,3-dithian-2-yl 2-methyl-1,3-oxathian-2-yl,
R³ represents hydrogen, chlorine, bromine, fluorine, iodine, OMe, methyl, 1,3-dioxolan-2yl, 4-methyl-1,3-dioxolan-2yl, 4-ethyl-1,3-dioxolan-2yl, 4,5-dimethyl-1,3-dioxolan-2-yl, 4-(methoxymethyl)-1,3-dioxolan-2-yl, tetrahydrofuro[3,4-d][1,3]dioxol-2-yl, 1,3-dioxolan-2-ylmethyl
1,3-dioxan-2-yl, 2-methyl-1,3-dioxan-2-yl, 4,6-dimethyl-1,3-dioxan-2-yl, 5,5-dimethyl-1,3-dioxan-2-yl,
1,3-dioxan-2-ylmethyl,
1,3-dithiolan-2-yl, 1,3-oxathiolan-2-yl, 1,3-dithian-2-yl, 1,3-oxathian-2-yl,
2-methyl-1,3-dioxolan-2-yl, 2-ethyl-1,3-dioxolan-2-yl, 2-(2,2,2-trifluoroethyl)-1,3-dioxan-2-yl, 2-[(2-methyl-1,3-dioxolan-2-yl)methyl]-1,3-dioxolan-2-yl, 2-(cyanomethyl)-1,3-dioxolan-2-yl,
(2-methyl-1,3-dioxolan-2-yl)methyl,
2-methyl-1,3-dioxan-2-yl, 2-ethyl-1,3-dioxan-2-yl, 2-(2,2,2-trifluoroethyl)-1,3-dioxan-2-yl,
2-[(2-methyl-1,3-dioxan-2-yl)methyl]-1,3-dioxan-2-yl, 2-(cyanomethyl)-1,3-dioxan-2-yl,
(2-methyl-1,3-dioxan-2-yl)methyl,
2-methyl-1,3-dithiolan-2-yl, 2-methyl-1,3-oxathiolan-2-yl, 2-methyl-1,3-dithian-2-yl or 2-methyl-1,3-oxathian-2-yl,
where R² and R³ are not both hydrogen,
with the proviso that, if R² is not hydrogen,
R³ can only have one of the meanings below:
hydrogen, chlorine, bromine, fluorine, iodine, OMe or methyl,
R⁴ represents hydrogen, chlorine, bromine, fluorine, iodine, OMe, SCF₃, methyl, ethyl, CF₃, CF₂CF₃ or CO₂Me,
R⁶ represents hydrogen,
R⁷ represents hydrogen,
R⁸ represents chlorine, bromine, fluorine, iodine, cyano, CF₃, methyl, SMe, S(O)Me or SO₂Me,
R⁹ represents hydrogen or methyl,
R¹⁰ represents cyclopropyl, cyclopropylmethyl, cyclobutyl, propan-2-yl, prop-2-yn-1-yl, 2,2,2-trifluoroethyl, 2,2-difluoroethyl or 1-methoxypropan-2-yl,
or
R⁹ and R¹⁰ together with the nitrogen atom to which they are attached represent an azetidinyl, pyrrolidinyl, piperidinyl or morpholinyl ring,
and agrochemically active salts thereof.

6. A composition for controlling phytopathogenic harmful fungi, comprising at least one compound of formula (I) according to claim 1, in addition to one or more extenders, surfactants or a combination thereof.

7. (canceled)

8. A method for controlling phytopathogenic harmful fungi comprising, applying the compound of the formula (I) according to claim 1 to one or more phytopathogenic harmful fungi, their habitat or a combination thereof.

9. A process for preparing a composition for controlling phytopathogenic harmful fungi comprising, mixing the compound of formula (I) according to claim 1 with one or more extenders, surfactants or a combination thereof.

10. A process for preparing the compound of formula (I) according to the invention 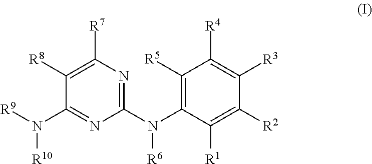
hereinbelow, depending on the appropriate process, also referred to by formulae (Ia-c) 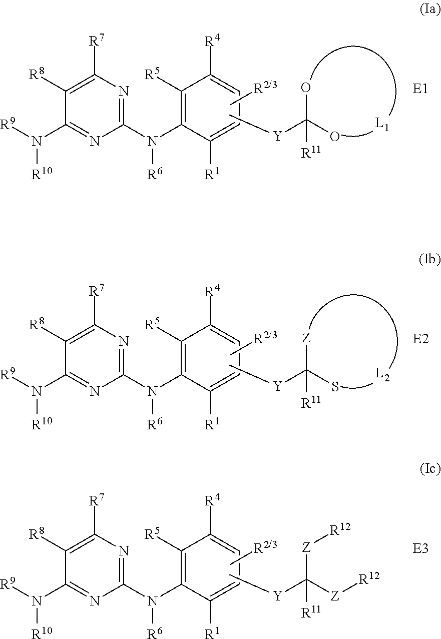
having in each case exactly one heterocyclic side chain E1, E2 or E3 in the R² or R³ position (meta or para),
comprising at least one of steps (a) to (g) below:
(a) reacting a compound of formula (III) with a compound of formula (II) in the presence of a base, if appropriate in the presence of a solvent, if appropriate in the presence of a catalyst, to give a compound of formula (V), according to the reaction scheme below: 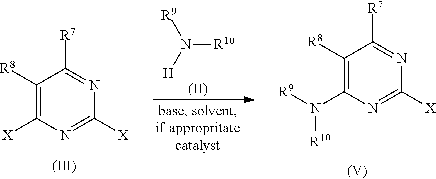
where X=F, Cl, Br or I
(b) reacting the compound of formula (V) with a compound of formula (IV), if appropriate in the presence of an acid, if appropriate in the presence of a solvent, according to the reaction scheme below: 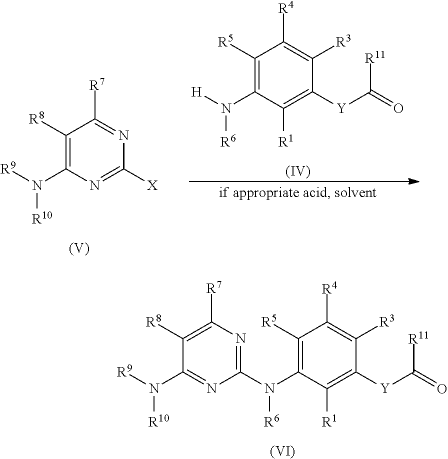
where X=F, Cl, Br or I
(c) reacting the compound of the formula (V) with a compound of formula (VII), if appropriate in the presence of an acid, if appropriate in the presence of a solvent, according to the reaction scheme below: 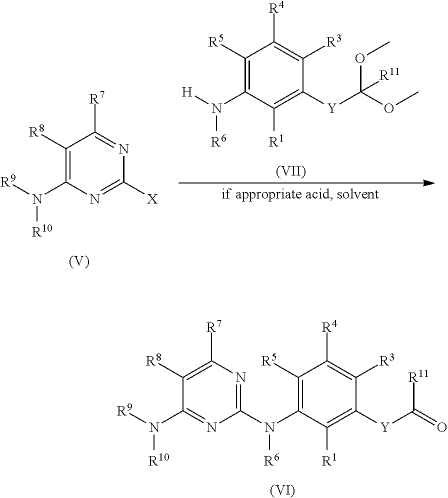
where X=F, Cl, Br or I
(d) reacting a compound of formula (VI) with a compound of formula (VIII) in the presence of an acid, if appropriate in the presence of a solvent, to give the compound of formula (Ia), according to the reaction scheme below: 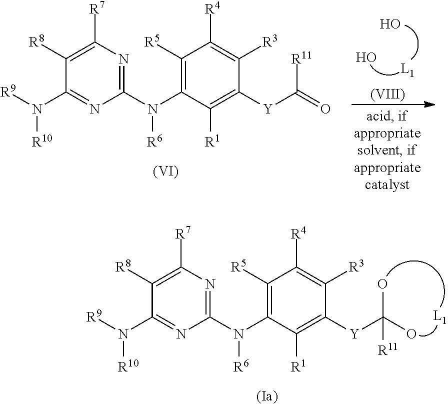
(e) reacting the compound of formula (V) with a compound of formula (IX) in the presence of an acid, if appropriate in the presence of a solvent, if appropriate in the presence of a catalyst, to give the compound of formula (Ia), according to the reaction scheme below: 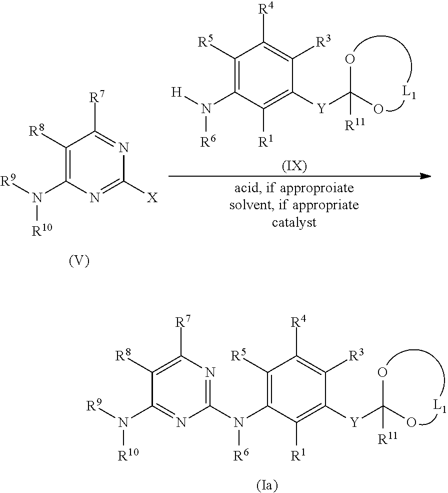
where X=F, Cl, Br or I
(f) reacting the compound of formula (VI) with a compound of formula (X) in the presence of an acid, if appropriate in the presence of a solvent, if appropriate in the presence of a catalyst, to give the compound of formula (Ib), according to the reaction scheme below: 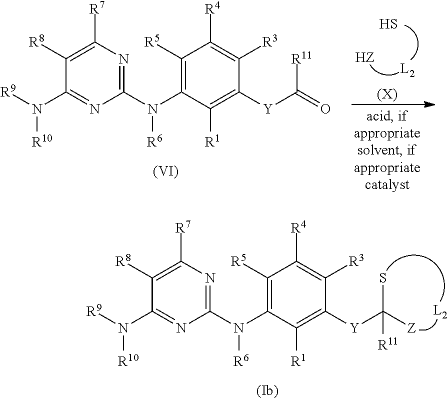
(g) reacting the compound of formula (VI) with a compound of formula (XI) in the presence of an acid, if appropriate in the presence of a solvent, if appropriate in the presence of a catalyst, to give the compound of formula (Ic), according to the reaction scheme below: 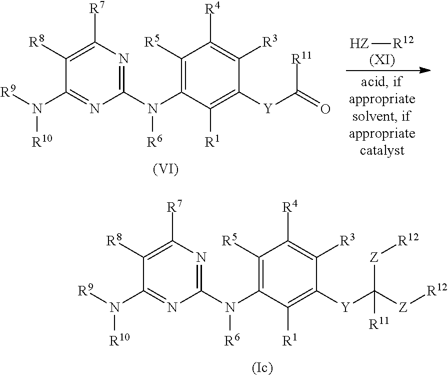
wherein R¹ to R¹², Y, and Z are defined according to claim 1.

11. The compound of the formula (VI) 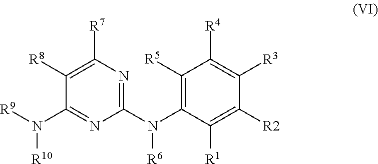
in which
R¹ and R⁵ represent hydrogen,
R² to R⁴ independently of one another represent hydrogen, OH, halogen, cyano, C₁-C₄ alkyl, C₁-C₄-alkoxy, C₁-C₄-haloalkyl, C₁-C₄-alkoxycarbonyl, NMe₂, SCF₃, SCH₃, OCF₂H or OCF₃,
where exactly one of the radicals R² and R³ represents a group of the formula E4 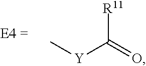
and in which the other symbols have the meanings below:
R¹¹ represents hydrogen, CH₂OH, optionally branched C₁-C₄-alkyl, optionally branched C₁-C₄-alkoxy-C₁-C₄-alkyl, optionally branched C₁-C₄-haloalkyl, C₁-C₄-alkoxycarbonylmethyl, optionally branched C₃-C₆-cycloalkyl, CH₂CN, CH₂CH₂CN, 3-oxobutanoyl, thiophen-2-yl, CH₂CH₂OH, phenyl or CH₂Ph,
R¹⁰ represents methyl, ethyl, propyl, cyclopropyl, 1-cyclopropyleth-1-yl, 2-methylcyclopropyl, 2,2-dimethylcyclopropyl, 2,2-dimethylprop-1-yl, tert-butyl, cyclobutyl, 2-methylcyclobut-1-yl, 3-methylcyclobut-1-yl, butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, butan-2-yl, 2,2,2-trifluoroethyl, 2,2-difluoroethyl, 2-methoxyethan-1-yl, 2-methylmercaptoethan-1-yl, 2-fluoroethan-1-yl, 2-chloroethan-1-yl, 2-cyanoethan-1-yl, 1-methoxypropan-2-yl, 3-methoxypropan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-1-yl, 1,1,1-trifluorobutan-2-yl, 1,1,1-trifluorobutan-3-yl, 2-methylprop-2-en-1-yl or 1-fluoropropan-2-yl,
or
R⁹ and R¹⁰ together with the nitrogen atom to which they are attached form an optionally branched, optionally substituted 3- to 7-membered saturated cycle which may contain up to one further heteroatom,
where the substituents independently of one another are selected from the group consisting of methyl, fluorine, chlorine and/or bromine atoms, cyano, hydroxyl, methoxy and CF₃,
and
Y, R⁶, R⁷, R⁸ and R⁹ as defined according to claim 1,
and agrochemically active salts thereof.

12. A composition for controlling phytopathogenic harmful fungi comprising, combining at least one compound of the formula (VI) according to claim 11 and one or more extenders, surfactants or a combination thereof.

13. (canceled)

14. A method for controlling phytopathogenic harmful fungi comprising, applying the compound of formula (VI) according to claim 11 to one or more phytopathogenic harmful fungi, their habitat or a combination thereof.

15. The compound of formula (V) 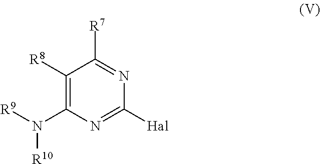
in which
R⁷ represents hydrogen,
and, if
R⁸ represents CF₃, CFH₂ or CF₂H,
Hal represents F, Cl, Br or I,
R⁹ represents hydrogen, ethyl, propyl, propan-2-yl, 2-methoxyethan-1-yl, prop-2-en-1-yl, CH₂OCH₃, COMe, COOMe, COOEt, COOtertBu, COCF₃ or benzyl,
R¹⁰ represents ethyl, propyl, cyclopropyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, 2-methylcyclobut-1-yl, 3-methylcyclobut-1-yl, butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, butan-2-yl, 2,2,2-trifluoroethyl, 2,2-difluoroethyl, 2-methoxyethan-1-yl, 2-methylmercaptoethan-1-yl, 2-fluoroethan-1-yl, 2-chloroethan-1-yl, 2-cyanoethan-1-yl, 1-methoxypropan-2-yl, 3-methoxypropan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1-yl or 1-fluoropropan-2-yl,
or
R⁹ and R¹⁰ together with the nitrogen atom to which they are attached form an unsubstituted or substituted 3- to 7-membered saturated cycle which may contain up to one further heteroatom,
where the substituents independently of one another are selected from the group consisting of methyl, fluorine, chlorine and/or bromine atoms, cyano, hydroxyl, methoxy, and CF₃,
and where the heteroatoms are selected from the group consisting of oxygen, sulphur and nitrogen.

16. A compositions for controlling phytopathogenic harmful fungi, comprising at least one compound of formula (I) according to claim 2, in addition to one or more extenders, surfactants or a combination thereof.

17. A compositions for controlling phytopathogenic harmful fungi, comprising at least one compound of formula (I) according to claim 3, in addition to one or more extenders, surfactants or a combination thereof.

18. A compositions for controlling phytopathogenic harmful fungi, comprising at least one compound of formula (I) according to claim 4, in addition to one or more extenders, surfactants or a combination thereof.

19. A compositions for controlling phytopathogenic harmful fungi, comprising at least one compound of formula (I) according to claim 5, in addition to one or more extenders, surfactants or a combination thereof.

20. A method for controlling phytopathogenic harmful fungi comprising, applying the compound of formula (I) according to claim 2 to one or more phytopathogenic harmful fungi, their habitat or a combination thereof.

21. A method for controlling phytopathogenic harmful fungi comprising, applying the compound of formula (I) according to claim 3 to one or more phytopathogenic harmful fungi, their habitat or a combination thereof.

22. A method for controlling phytopathogenic harmful fungi comprising, applying the compound of formula (I) according to claim 4 to one or more phytopathogenic harmful fungi, their habitat or a combination thereof.

23. A method for controlling phytopathogenic harmful fungi comprising, applying the compound of formula (I) according to claim 5 to one or more phytopathogenic harmful fungi, their habitat or a combination thereof.
